SEQUENCE LISTING (9 sequences)

SEQ ID NO: 1 - DNA, 3579 bp, Homo sapiens
atgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtga

SEQ ID NO: 2 - DNA, 810 bp, Artificial Sequence
  Adeno-Associated Virus
gtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgccc

SEQ ID NO: 3 - DNA, 8562 bp, Artificial Sequence
  Adeno-Associated Virus
gcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 4 - DNA, 564 bp, Artificial Sequence
  Adeno-Associated Virus
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 5 - DNA, 8409 bp, Artificial Sequence
  Adeno-Associated Virus
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagacagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 6 - DNA, 8611 bp, Artificial Sequence
  Adeno-Associated Virus
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 7 - DNA, 792 bp, Artificial Sequence
  Adeno-Associated Virus
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 8 - DNA, 8629 bp, Artificial Sequence
  Kanamycin Plasmid
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagct

SEQ ID NO: 9 - DNA, 4977 bp, Artificial Sequence
  Kanamycin Cassette
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

(19) United States
(12) Patent Application Publication
(10) Pub. No.: US 2025/0018059 A1
(43) Pub. Date: Jan. 16, 2025

(54) PRODUCTION OF RECOMBINANT AAV VECTORS FOR TREATING MUSCULAR DYSTROPHY

(71) Applicant: Sarepta Therapeutics, Inc., Cambridge, MA (US)

(72) Inventors: Maroof ALAM, Cambridge, MA (US); Louise RODINO-KLAPAC, Cambridge, MA (US)

(73) Assignee: Sarepta Therapeutics, Inc., Cambridge, MA (US)

(21) Appl. No.: 18/562,148

(22) PCT Filed: May 13, 2022
(86) PCT No.: PCT/US2022/029328

§ 371 Date: Nov. 17, 2023

Related U.S. Application Data
(60) Provisional application No. 63/189,676, filed on May 17, 2021.
(60) Provisional application No. 63/209,733, filed on Jun. 11, 2021.
(60) Provisional application No. 63/243,944, filed on Sep. 14, 2021.
(60) Provisional application No. 63/253,998, filed on Oct. 8, 2021.

(51) Int. Cl. A61K 48/00; A61P 21/00; C07K 14/47; C12N 15/86

(52) U.S. Cl. CPC A61K 48/0058; A61P 21/00; C07K 14/4708; C12N 15/86; C12N 2750/14122; C12N 2750/14143; C12N 2750/14152; C12N 2750/14171

(57) ABSTRACT

The present disclosure provides gene therapy vectors, such as recombinant adeno-associated virus (rAAV), produced in mammalian adherent cells cultured in suspension conditions, for expressing a human micro-dystrophin gene. The present disclosure also provides compositions and methods of using these rAAV to treat muscular dystrophy, such as, e.g., Duchenne Muscular Dystrophy.

### CROSS-REFERENCE TO RELATED APPLICATIONS

This application claims the benefit of U.S. Provisional Appl. No. 63/253,998, filed on Oct. 8, 2021, U.S. Provisional Appl. No. 63/243,944, filed on Sep. 14, 2021, U.S. Provisional Appl. No. 63/209,733, filed on Jun. 11, 2021, and U.S. Provisional Appl. No. 63/189,676, filed on May 17, 2021, which are hereby incorporated by reference in their entirety.

### REFERENCE TO SEQUENCE LISTING SUBMITTED ELECTRONICALLY

The content of the electronically submitted sequence listing in ASCII text file (Name: 4140_052PC04_Seglisting_ST25.txt; Size: 60,194 bytes; and Date of Creation: May 13, 2022), filed with the application, is incorporated herein by reference in its entirety.

### FIELD

The present disclosure is in the field of gene therapy. More particularly, the present disclosure provides gene therapy vectors, such as adeno-associated virus (AAV) vectors, for expressing a miniaturized human micro-dystrophin gene, wherein the AAVs are produced from adherent cells cultured in suspension conditions. The present disclosure also provides methods of using these vectors to express micro-dystrophin in skeletal muscles, including diaphragm and cardiac muscle, and to protect muscle fibers from injury, increase muscle strength, and reduce and/or prevent fibrosis in subjects suffering from muscular dystrophy.

### BACKGROUND

The importance of muscle mass and strength for daily activities, such as locomotion and breathing, and for whole body metabolism is unequivocal. Deficits in muscle function produce muscular dystrophies (MDs) that are characterized by muscle weakness and wasting and have serious impacts on quality of life. The most well-characterized MDs result from mutations in genes encoding members of the dystrophin-associated protein complex (DAPC). These MDs result from membrane fragility associated with the loss of sarcolemmal-cytoskeleton tethering by the DAPC. Duchenne Muscular Dystrophy (DMD) is one of the most devastating muscle disease affecting 1 in 5,000 newborn males.

DMD is caused by mutations in the DMD gene leading to reductions in mRNA and the absence of dystrophin, a 427 kD sarcolemmal protein associated with the dystrophin-associated protein complex (DAPC) (Hoffman et al., Cell 51:919-28, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction and protect against contraction-induced damage. With dystrophin loss, membrane fragility results in sarcolemmal tears and an influx of calcium, triggering calcium-activated proteases and segmental fiber necrosis (Straub et al., Curr Opin. Neurol. 10: 168-75 (1997)). This uncontrolled cycle of muscle degeneration and regeneration ultimately exhausts the muscle stem cell population (Sacco et al., Cell 143: 1059-1071 (2010); Wallace et al., Annu Rev Physiol 71: 37-57 (2009)), resulting in progressive muscle weakness, endomysial inflammation, and fibrotic scarring.

Without membrane stabilization from dystrophin or a micro-dystrophin, DMD will manifest as uncontrolled cycles of tissue injury and repair, ultimately replacing lost muscle fibers with fibrotic scar tissue through connective tissue proliferation. Fibrosis is characterized by the excessive deposits of ECM matrix proteins, including collagen and elastin. ECM proteins are primarily produced from cytokines such as TGFβ that are released by activated fibroblasts responding to stress and inflammation. Although the primary pathological feature of DMD is myofiber degeneration and necrosis, fibrosis as a pathological consequence has equal repercussions. The over-production of fibrotic tissue restricts muscle regeneration and contributes to progressive muscle weakness in the DMD patient. In one study, the presence of fibrosis on initial DMD muscle biopsies was highly correlated with poor motor outcome at a 10-year follow-up (Desguerre et al., J Neuropathol Exp Neurol 68: 762-767 (2009)). These results point to fibrosis as a major contributor to DMD muscle dysfunction and highlight the need for early intervention prior to overt fibrosis.

International Publ. No. WO 2019/245973 A1, incorporated by reference herein in its entirety, describes AAV vector delivery of the micro-dystrophin gene to treat muscular dystrophy (e.g., DMD) in a human subject, There is a continued need in the art, however, for improved methods of producing such AAV vectors, especially methods suitable for large-scale manufacture of such gene therapy vectors.

### SUMMARY

The present disclosure is directed to gene therapy vectors, e.g. AAV vectors, produced by a suspension seed process described herein that expresses the human micro-dystrophin gene in skeletal muscles, including diaphragm and cardiac muscle, to protect muscle fibers from injury, increase muscle strength, and reduce and/or prevent fibrosis.

The present disclosure provides a method of producing a recombinant adeno-associated virus (rAAV) rAAVrh74.MHCK7.microdystrophin in adherent mammalian cells by a suspension seed process, comprising: (a) culturing cells with a first growth medium comprising serum in a N-2 container; (b) removing the cells from the first medium; (c) inoculating the cells from step (b) into a second medium comprising no serum or serum at a concentration less than the first medium in a N-1 container; (d) culturing the cells in the N-1 container under suspension conditions; and (e) inoculating a third medium in a bioreactor with the cells from step (d).

In some aspects, the rAAV used in the method described herein comprises the human micro-dystrophin nucleotide sequence of SEQ ID NO:1. In some aspects, the rAAV comprises the MHCK7 promoter sequence of SEQ ID NO: 7. In some aspects, the rAAV comprises the human micro-dystrophin nucleotide sequence of SEQ ID NO:1 and the MHCK7 promoter sequence of SEQ ID NO: 7.

In some aspects, the suspension seed process further comprises: (f) transfecting the adherent cells with a transgene plasmid comprising a rAAVrh74.MHCK7.microdystrophin construct, a plasmid comprising an AAV rep gene and an AAV cap gene, and an adenovirus helper plasmid.

In some aspects, the transgene plasmid comprising a rAAVrh74.MHCK7.microdystrophin construct comprises: the nucleic acid sequence of SEQ ID NO: 9; nucleotides 55-5021 of SEQ ID NO: 3; or nucleotides 1-4977 of SEQ ID NO: 8. In some aspects, the plasmid comprising an AAV rep gene and an AAV cap gene comprises an AAV2 rep gene and an rAAVrh74 cap gene. In some aspects, the adenovirus helper plasmid comprises an adenovirus 5 E2A, E4ORF6, and a VA RNA gene.

In some aspects, the suspension seed process further comprises: (g) lysing the adherent cells. In some aspects, the adherent cells are lysed by freeze-thaw, solid shear, hypertonic and/or hypotonic lysis, liquid shear, sonication, high-pressure extrusion, detergent lysis, or combinations thereof.

In some aspects, the suspension seed process further comprises (h) purifying the rAAV by at least one column chromatography step. In some aspects, the at least one column chromatography step comprises an anion exchange chromatography, a size exclusion chromatography, or a combination thereof.

In some aspects, the suspension seed process further comprises culturing cells with the first growth medium in a N-3 container. In some aspects, the suspension seed process further comprises culturing cells with the first growth medium in a N-4 container.

In some aspects, the bioreactor is an adherent bioreactor. In some aspects, the rAAV is purified from the culture produced in the adherent bioreactor.

In some aspects, the third medium in the bioreactor comprises at least one factor that promotes cell adherence. In some aspects, the at least one factor that promotes cell adherence is selected from the group consisting of serum, FBS, fibronectin, collagen, laminin, calcium ions, proteoglycans or non-proteoglycan polysaccharides of the extracellular matrix, and combinations thereof. In some aspects, the third medium in the bioreactor comprises DMEM and 10% FBS.

In some aspects, the adherent cells are cultured under suspension conditions for about 48-72 hours.

In some aspects, the N-1 container is a suspension shake flask.

In some aspects, the adherent cells are selected from the group consisting of HeLa cells, CHO cells, HEK-293 cells, VERO cells, BHK cells, MDCK cells, MDBK cells, and COS cells. In some aspects, the adherent cells are HeLa cells or HEK-293 cells. In some aspects, the adherent cells are HEK-293 cells. In some aspects, the adherent cells are not suspension-adapted. In some aspects, culturing the cells under suspension conditions does not alter the adherent-dependency of the cells. In some aspects, the culturing does not alter the cells to create a new cell line.

The present disclosure also provides a composition comprising a recombinant adeno-associated virus (rAAV) rAAVrh74.MHCK7.microdystrophin, wherein the rAAV is made by any of the methods described herein. In some aspects, the composition comprises: a) rAAV particles comprising the nucleic acid sequence of SEQ ID NO: 9; b) rAAV particles comprising nucleotides 55-5021 of SEQ ID NO: 3; and/or c) rAAV particles comprising nucleotides 1-4977 of SEQ ID NO: 8.

In some aspects, the present disclosure provides a composition for treating muscular dystrophy in a subject in need thereof, comprising a recombinant adeno-associated virus (rAAV) rAAV.rh74MHCK7.microdystrophin, wherein the rAAV is produced in adherent cells, and wherein the adherent cells are cultured in an N-1 container under suspension conditions. In some aspects, the rAAV comprises the human micro-dystrophin nucleotide sequence of SEQ ID NO:1. In some aspects, the rAAV comprises the MHCK7 promoter sequence of SEQ ID NO: 7. In some aspects, the rAAV comprises the MHCK7 promoter sequence of SEQ ID NO: 7 and the human micro-dystrophin nucleotide sequence of SEQ ID NO: 1.

In some aspects, the composition comprises: (a) rAAV comprising the nucleic acid sequence of SEQ ID NO: 9; (b) rAAV particles comprising the nucleic acid sequence of SEQ ID NO: 9; (c) rAAV comprising nucleotides 55-5021 of SEQ ID NO: 3; (d) rAAV particles comprising nucleotides 55-5021 of SEQ ID NO: 3; (e) rAAV comprising nucleotides 1-4977 of SEQ ID NO: 8; and/or (f) rAAV particles comprising nucleotides 1-4977 of SEQ ID NO: 8.

The present disclosure also provides a method of treating muscular dystrophy in a human subject in need thereof comprising administering the composition comprising the rAAV described herein to said human subject. In some aspects, the rAAV is administered using a systemic route of administration and at a dose of about 5.0×10¹² vg/kg to about 1.0×10¹⁵ vg/kg. In some aspects, the systemic route of administration is an intravenous route and the dose of the rAAV administered is about 2×10¹⁴ vg/kg.

In some aspects, the dose of rAAV is administered at a concentration of about 10 mL/kg. In some aspects, the rAAV is administered by injection, infusion, or implantation. In some aspects, the rAAV is administered by infusion over approximately one hour. In some aspects, the rAAV is administered by an intravenous route through a peripheral limb vein.

In some aspects, the muscular dystrophy is Duchenne muscular dystrophy or Becker's muscular dystrophy. In some aspects, the muscular dystrophy is Duchenne muscular dystrophy.

In some aspects, the level of micro-dystrophin gene expression in a cell of the subject is increased after administration of the rAAV as compared to the level of micro-dystrophin gene expression before administration of the rAAV. In some aspects, expression of the micro-dystrophin gene in the cell is detected by measuring the micro-dystrophin protein level by Western blot in muscle biopsied before and after administration of the rAAV. In some aspects, expression is at least 55.4% after administration of the rAAV, as compared to before.

In some aspects, the mean percentage of micro-dystrophin positive fibers in the muscle tissue of the subject is increased after administration of the rAAV, as compared to the number of micro-dystrophin positive fibers before administration of the rAAV. In some aspects, the mean percentage of micro-dystrophin positive fibers is at least 70.5% and the mean intensity is at least 116.9% as detected by immunofluorescence (IF) in muscle biopsies before and after administration of the rAAV. In some aspects, micro-dystrophin transduction by vector genome count is at least 3.87 mean vector genome copies per nucleus.

In some aspects, the composition is administered to a genotyped patient. In some aspects, the patient's human dystrophin (DMD) gene is genotyped. In some aspects, the genotyped patient is genotyped for at least one mutation in exons 18-79 of the human dystrophin (DMD) gene.

In some aspects, the method of treating muscular dystrophy further comprises genotyping the DMD gene of the human subject prior to administering the composition to said human subject. In some aspects, the genotyping detects at least one mutation in exons 18 to 79 of the DMD gene. In some aspects, the at least one mutation is a frameshift deletion, a frameshift duplication, a premature stop, or other pathogenic variant resulting in the absence of expression of the human dystrophin protein.

The present disclosure also provides a use of the composition described herein for the treatment of muscular dystrophy in a human subject in need thereof. In some aspects, the present disclosure also provides a use of the composition described herein in the manufacture of a medicament for the treatment of muscular dystrophy.

In some aspects, the muscular dystrophy is Duchenne muscular dystrophy or Becker's muscular dystrophy. In some aspects, the muscular dystrophy is Duchenne muscular dystrophy.

### BRIEF DESCRIPTION OF THE DRAWINGS

FIG. 1 illustrates the rAAV.MHCK7.micro-dystrophin construct. In this construct, the cDNA expression cassette is flanked by AAV2 inverted terminal repeat sequences (ITR). The construct is characterized by an in-frame rod deletion (R4-R23), while hinges 1, 2, and 4 (H₁, H₂, and H₄) and the cysteine rich domain remain producing a 138 kDa protein. The expression of the micro-dystrophin protein (3579 bp) is guided by a MHCK7 promoter (795 bp). The intron and 5′ UTR are derived from plasmid pCMVβ (Clontech). The micro-dystrophin cassette had a consensus Kozak immediately in front of the ATG start and a small 53 bp synthetic polyA signal for mRNA termination. The human micro-dystrophin cassette contained the (R4-R23/Δ71-78) domains as previously described by Harper et al. (Nature Medicine 8: 253-261 (2002)).

FIG. 2 provides the nucleic acid sequence (SEQ ID NO: 3) of AAVrh74.MHCK7.micro-dystrophin.

FIG. 3 provides the pNLREP2-Caprh74 AAV helper plasmid map.

FIG. 4 provides the Ad Helper plasmid pHELP.

FIG. 5 illustrates the rAAV.MCK.micro-dystrophin plasmid construct.

FIG. 6 provides the nucleic acid sequence (SEQ ID NO: 5) of rAAVrh74.MCK.micro-dystrophin.

FIG. 7 demonstrates micro-dystrophin gene expression in muscle fibers of gastrocnemius muscle biopsy as measured by immunocytochemistry.

FIGS. 8A-8C provide Western blots demonstrating micro-dystrophin protein expression at the correct molecular weight. In FIGS. 8A and 8B, Western blot analysis detected micro-dystrophin protein expression in Subject 1 (age 5), Subject 2 (age 4), and Subject 3 (age 6). In FIG. 8C, Subject 4 samples (*) were diluted 1:4 (to linear range) as ULDQ (>80%) exceeded in initial analysis, and mean values were multiplied by the dilution correction factor for final value in comparison to normal. Mean Micro-dystrophin Expression vs. Normal was 182.7% in Method 1 and 222.0% in Method 2.

FIGS. 9A-9C demonstrate that administration of rAAVrh74.MHCK7. micro-dystrophin upregulates expression of the DAPC proteins, alpha-sarcoglycan and beta-sarcoglycan, in Subject 1 (FIG. 9A), Subject 2 (FIG. 9B), and Subject 3 (FIG. 9C).

FIG. 10 provides a graph showing a sustained dramatic reduction in Creatine Kinase (CK) values with administration of rAAVrh74.MHCK7.micro-dystrophin.

FIG. 11 provides a graph showing the mean creatine kinase (CK) change from baseline to day 270. This data demonstrate that CK significantly decreased over time after administration of rAAVrh74.MHCK7.micro-dystrophin.

FIG. 12 provides a graph showing the mean NSAA change and the mean CK change from baseline to day 270. This data demonstrate that NSAA significantly increased over time after administration of rAAVrh74.MHCK7.micro-dystrophin.

FIG. 13 provides the 4977 base nucleic acid sequence (SEQ ID NO: 9) of the AAVrh74.MHCK7.micro-dystrophin construct. The following molecular elements are delineated: 5′ ITR (bases 1-145); MHCK7 promoter (190-981 (792 bases)); Intron (991-1140 (150 bases)); human micro-dystrophin sequence (1151-4729 (3579 bases)); PolyA tail (4732-4784 (53 bases)); and 3′ ITR (4833-4977 (145 bases)).

FIG. 14 illustrates the AAVrh74.MHCK7.micro-dystrophin plasmid construct.

FIG. 15 provides the nucleic acid sequence (SEQ ID NO: 8) of the AAVrh74.MHCK7.micro-dystrophin plasmid construct, which comprises the kanamycin resistance gene.

FIG. 16 is a visual representation of the hybrid seed-train expansion method, as described herein.

FIG. 17 is a visual representation of producing an AAV particle using the hybrid seed-train expansion method, as described herein.

FIGS. 18A-18B provide graphs showing the viability of HEK-293 cells cultured according to the hybrid seed-train expansion method disclosed herein (FIG. 18A) and under adherent conditions only (FIG. 18B).

FIGS. 19A-19B provide graphs showing the viable cell density of HEK-293 cells cultured according to the hybrid seed-train expansion method disclosed herein (FIG. 19A) and under adherent conditions only (FIG. 19B).

FIG. 20 is a graph showing the mean NSAA Score from Cohort 1 (first 11 patients treated with rAAVrh74.MHCK7.microdystrophin) described in Example 7. The first 11 patients improved 3 points from baseline. 6 to 7 year olds (n=9) improved 2.9 points from baseline. Each time point represents the 11 patients.

FIGS. 21A-21C demonstrate expression of micro-dystrophin in skeletal and cardiac muscle (immunofluorescence) of DMD^{mdx} rats at 12 weeks (FIG. 21B) and 24 weeks (FIG. 21C) after treatment with delandistrogene moxeparvovec, compared to saline (FIG. 21A), as discussed in Example 11. Key: LTA=left tibialis anterior; HRT=heart.

FIGS. 22A-22B are bar graphs depicting the quantitation of micro-dystrophin expression (immunofluorescence) (FIG. 22A) and vector transduction (vector genome copies) (FIG. 22B) in muscle tissues of DMD^{mdx} rats at 12 weeks and 24 weeks after treatment with delandistrogene moxeparvove, as discussed in Example 11. Key: TA=tibialis; HRT=heart; MG=medial gastrocnemius; LG=lateral medial gastrocnemius; DIA=diaphragm; TRI=triceps; PSO=psoas major.

FIGS. 23A-23B are bar graphs depicting an increase in ambulation (FIG. 23A) and vertical activity (FIG. 23B) in DMD^{mdx} rats at 12 weeks and 24 weeks after treatment with delandistrogene moxeparvovec, compared to saline, as discussed in Example 11. Movement of the rats (ambulation and vertical activity) in activity cages were measured by laser light beam breaks per hour. Each point represents a value for an individual animal. Data are reported as mean±SD; ***=p<0.001; **=p<0.01. SD=standard deviation.

FIGS. 24A-24B depict a significant reduction in muscle degeneration by central nucleation analysis in skeletal muscle at 12 weeks and 24 weeks after delandistrogene moxeparvovec gene transfer in DMD^{mdx} rats, compared to saline, as discussed in Example 11. FIG. 24A depicts Hematoxylin and Eosin (H & E) staining of gastrocnemius muscle. FIG. 24B are bar graphs depicting the percentage of fibers with central nuclei. Bars are reported as mean±SD; ****=p<0.0001. SD=standard deviation.

FIGS. 25A-25B depict an analysis of collagen deposition in skeletal and cardiac muscle showing reduced fibrosis after treatment with delandistrogene moxeparvovec in DMD^{mdx} rats at 12 weeks and 24 weeks, compared to saline, as discussed in Example 11. FIG. 25A depicts Masson's Trichome staining at 12 weeks post-treatment. FIG. 25B provides bar graphs quantifying collagen deposition in skeletal and cardiac muscle for 12 and 24 weeks post-treatment. Key: HRT=heart; MG=medial gastrocnemius; DIA=diaphragm. Data are reported as mean±SD; ****=p<0.0001; *=p<0.05. SD=standard deviation.

FIG. 26 is a bar graph showing that serum troponin I levels in blood do not change significantly 1 week and 12 weeks after treatment with delandistrogene moxeparvovec in DMD^{mdx} rats, compared to saline, as discussed in Example 11. Bars represent the mean+SD. Each point represents a value for an individual animal.

FIGS. 27A-27C are bar graphs analyzing cardiac function, as determined by echocardiography, in DMD^{mdx} rats 24 weeks after treatment with delandistrogene moxeparvovec, compared to saline, as discussed in Example 11. FIG. 27A depicts data for left ventricular end-systolic diameter (LVESD). FIG. 27B depicts data for ejection fraction (%) (EF). FIG. 27C depicts data for fractional shortening (%) (FS).

### DETAILED DESCRIPTION

The present disclosure provides for gene therapy vectors, e.g., rAAV, that express human micro-dystrophin, wherein the rAAV are produced in mammalian adherent cells, and wherein the adherent cells are cultured in an N-1 container under suspension conditions.

The present disclosure provides a method of producing a recombinant adeno-associated virus (rAAV) rAAVrh74.MHCK7.microdystrophin in adherent mammalian cells by a suspension seed process, comprising: (a) culturing cells with a first growth medium comprising serum in a N-2 container; (b) removing the cells from the first medium; (c) inoculating the cells from step (b) into a second medium comprising no serum or serum at a concentration less than the first medium in a N-1 container; (d) culturing the cells in the N-1 container under suspension conditions; and (e) inoculating a third medium in a bioreactor with the cells from step (d).

The present disclosure also provides compositions (e.g., pharmaceutical compositions) comprising the rAAV disclosed herein, as well as methods of treating muscular dystrophy (e.g., DMD) using the compositions disclosed herein.

Muscle biopsies taken at the earliest age of diagnosis of DMD reveal prominent connective tissue proliferation. Muscle fibrosis is deleterious in multiple ways. It reduces normal transit of endomysial nutrients through connective tissue barriers, reduces the blood flow and deprives muscle of vascular-derived nutritional constituents, and functionally contributes to early loss of ambulation through limb contractures. Over time, treatment challenges multiply as a result of marked fibrosis in muscle. This can be observed in muscle biopsies comparing connective tissue proliferation at successive time points. The process continues to exacerbate leading to loss of ambulation and accelerating out of control, especially in wheelchair-dependent patients.

Without early treatment including a parallel approach to reduce fibrosis, it is unlikely that the benefits of exon skipping, stop-codon read-through, or gene replacement therapies can ever be fully achieved. Even small molecules or protein replacement strategies are likely to fail without an approach to reduce muscle fibrosis. Previous work in aged mdx mice with existing fibrosis treated with AAV.micro-dystrophin demonstrated that we could not achieve full functional restoration (Liu, M. et al., Mol Ther 11: 245-256 (2005)). It is also known that progression of DMD cardiomyopathy is accompanied by scarring and fibrosis in the ventricular wall.

### Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular.

Throughout this disclosure, the term “a” or “an” entity refers to one or more of that entity; for example, “a polynucleotide,” is understood to represent one or more polynucleotides. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower), unless indicated otherwise.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures.

Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with, 37 CFR § 1.822 and established usage.

“Polynucleotide” or “nucleic acid” as used herein means a sequence of nucleotides connected by phosphodiester linkages. Polynucleotides are presented herein in the direction from the 5′ to the 3′ direction. A polynucleotide of the present disclosure can be a deoxyribonucleic acid (DNA) molecule or ribonucleic acid (RNA) molecule. Nucleotide bases are indicated herein by a single letter code: adenine (A), guanine (G), thymine (T), cytosine (C), inosine (I) and uracil (U).

As used herein, the term “polypeptide” encompasses both peptides and proteins, unless indicated otherwise.

The term “coding sequence” or sequence “encoding” is used herein to mean a DNA or RNA region (the transcribed region) which “encodes” a particular protein, e.g., such as an insulin or a glucokinase. A coding sequence is transcribed (DNA) and translated (RNA) into a polypeptide, in vitro or in vivo, when placed under the control of an appropriate regulatory region, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotes or eukaryotes, genomic DNA from prokaryotes or eukaryotes, and synthetic DNA sequences. A transcription termination sequence can be located 3′ to the coding sequence.

A gene can comprise several operably linked fragments, such as a promoter, a 5′ leader sequence, an intron, a coding sequence and a 3′-nontranslated sequence, e.g., comprising a polyadenylation site or a signal sequence. As used herein, “expression of a gene” refers to the process wherein a gene is transcribed into an RNA and/or translated into an active protein.

As used herein, the term “promoter” refers to a nucleic acid sequence or fragment that functions to control the transcription of one or more genes (or coding sequence), located upstream with respect to the direction of transcription of the transcription initiation site of the gene, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most physiological and developmental conditions. An “inducible” promoter is a promoter that is regulated depending on physiological or developmental conditions. A “tissue specific” promoter is preferentially active in specific types of differentiated cells/tissues.

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region.

The term “operably linked” refers to the positioning of the regulatory element nucleotide sequence, e.g. promoter nucleotide sequence, to confer expression of said nucleotide sequence by said regulatory element.

As used herein, the term “transgene” refers to a gene (e.g., micro-dystrophin) or a nucleic acid molecule that is introduced into a cell. An example of a transgene is a nucleic acid encoding a therapeutic polypeptide. In some aspects, the gene can be present, but in some cases, the gene is normally not expressed or is expressed at an insufficient level in the cell. In this context, “insufficient” means that although said gene is normally expressed in a cell, a condition and/or disease could still be developed. In certain aspects, the transgene allows for the increased expression or over-expression of the gene. The transgene can comprise sequences that are native to the cell, comprise sequences that do not naturally occur in the cell, or it can comprise combinations of both. In certain aspects, the transgene can comprise a sequence that can be operably linked to appropriate regulatory sequences for expression of the gene. In some aspects, the transgene is not integrated into the host cell's genome.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

As used herein, the term “adeno-associated vector” or “AAV vector” refers to a vector comprising one or more polynucleotides of interest (or transgenes, such as micro-dystrophin) that are flanked by AAV terminal repeat sequences (ITRs). Such AAV vectors can be replicated and packaged into infectious viral particles when present in a host cell that has been transfected with a vector encoding and expressing rep and cap gene products.

As used herein, the terms “AAV virion,” “AAV viral particle,” or “AAV vector particle” refer to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of an AAV vector particle necessarily includes production of an AAV vector. As such, a vector is contained within an AAV vector particle.

The term “muscle specific control element” refers to a nucleotide sequence that regulates expression of a coding sequence that is specific for expression in muscle tissue. These control elements include enhancers and promoters. The present disclosure provides for constructs comprising the muscle specific control elements MCKH7 promoter, the MCK promoter and the MCK enhancer.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes, and cardiomyoblasts.

As used herein, the term “transduction” refers to the administration/delivery of the coding region of the micro-dystrophin to a recipient cell either in vivo or in vitro, via a replication-deficient rAAV of the present disclosure resulting in expression of micro-dystrophin by the recipient cell.

As used herein, the term “transfection” of a cell means that genetic material is introduced into a cell for the purpose of genetically modifying the cell. Transfection can be accomplished by a variety of means known in the art, e.g., transduction or electroporation.

As used herein, “vector” refers to a recombinant plasmid or virus that comprises a polynucleotide to be delivered into a host cell, either in vitro or in vivo. “Recombinant” means distinct from that generally found in nature.

“Serotype,” with respect to a vector or virus capsid, is defined by a distinct immunological profile based on the capsid protein sequences and capsid structure.

“AAV Cap” means AAV Cap proteins, VP1, VP2, and VP3 and analogs thereof.

“AAV Rep” means AAV Rep proteins and analogs thereof.

As used herein, “flanked,” with respect to a sequence that is flanked by other elements, indicates the presence of one or more the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the transgene and a flanking element. A sequence (e.g., a transgene) that is “flanked” by two other elements (e.g., ITRs), indicates that one element is located 5′ to the sequence and the other is located 3′ to the sequence; however, there may be intervening sequences between.

As used herein, the term “gene therapy” refers to the insertion of nucleic acid sequences (e.g., a nucleic acid comprising a promoter operably linked to a polynucleotide encoding a transgene, e.g., micro-dystrophin) into an individual's cells and/or tissues to treat a disease or condition. Such transgenes can be exogenous. An exogenous molecule or sequence is understood to be a molecule or sequence not normally occurring in the cell, tissue and/or individual to be treated.

As used herein, the term “genotyping” refers to a process of determining the specific allelic composition of a cell and/or subject at one or more positions within the genome, e.g. by determining the nucleic acid sequence at that position. Genotyping refers to a nucleic acid analysis and/or analysis at the nucleic acid level. In some aspects, the human dystrophin gene (DMD) of a subject is genotyped to characterize a mutation in the gene that would be especially amenable to treatment with the compositions disclosed herein. Numerous genotyping techniques are known to those skilled in the art.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, Cold Spring Harbor, N.Y. (1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos). Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO4 (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson, M. L. M. et al., Nucleic Acid Hybridisation: A Practical Approach, Ch. 4, IRL Press Limited (Oxford, England)(1998). Hybridization conditions can be adjusted by one skilled in the art in order to accommodate these variables and allow DNAs of different sequence relatedness to form hybrids.

As used herein, the terms “media”, “medium,” “cell culture medium,” “culture medium,” “tissue culture medium,” “tissue culture media,” and “growth medium” refer to a solution containing nutrients which nourish growing cultured eukaryotic cells. Typically, these solutions provide essential and non-essential amino acids, vitamins, energy sources, lipids, and trace elements required by the cell for minimal growth and/or survival. The solution can also contain components that enhance growth and/or survival above the minimal rate, including hormones and growth factors. The solution is formulated to a pH and salt concentration optimal for cell survival and proliferation. The medium can also be a “defined medium” or “chemically defined medium” a serum-free medium that contains no proteins, hydrolysates or components of unknown composition. Defined media are free of animal-derived components and all components have a known chemical structure. One of skill in the art understands a defined medium can comprise recombinant glycoproteins or proteins, for example, but not limited to, hormones, cytokines, interleukins and other signaling molecules.

As used herein, the term “basal media formulation” or “basal media” refers to any cell culture media used to culture cells that has not been modified either by supplementation, or by selective removal of a certain component.

As used herein, the terms “culture,” “cell culture,” and “eukaryotic cell culture” refer to a eukaryotic cell population, either surface-attached (i.e., adherent) or in suspension, that is maintained or grown in a medium under conditions suitable to survival and/or growth of the cell population. As will be clear to those of ordinary skill in the art, these terms as used herein can refer to the combination comprising the mammalian cell population and the medium in which the population is suspended.

As used herein, the term “batch culture” refers to a method of culturing cells in which all the components that will ultimately be used in culturing the cells, including the medium as well as the cells themselves, are provided at the beginning of the culturing process. A batch culture is typically stopped at some point and the cells and/or components in the medium are harvested and optionally purified.

As used herein, the term “fed-batch culture” refers to a method of culturing cells in which additional components are provided to the culture at some time subsequent to the beginning of the culture process. A fed-batch culture can be started using a basal medium. The culture medium with which additional components are provided to the culture at some time subsequent to the beginning of the culture process is a feed medium. The provided components typically comprise nutritional supplements for the cells which have been depleted during the culturing process. A fed-batch culture is typically stopped at some point and the cells and/or components in the medium are harvested and optionally purified.

As used herein, the term “perfusion culture” refers to a method of culturing cells in which additional components are provided continuously or semi-continuously to the culture subsequent to the beginning of the culture process. The provided components typically comprise nutritional supplements for the cells which have been depleted during the culturing process. A portion of the cells and/or components in the medium are typically harvested on a continuous or semi-continuous basis and are optionally purified.

“Growth phase” of the cell culture refers to the period of exponential cell growth (the log phase) where cells are generally rapidly dividing. During this phase, cells are cultured for a period of time, usually between 1-4 days, and under such conditions that cell growth is maximized. The determination of the growth cycle for the host cell can be determined for the particular host cell envisioned without undue experimentation. “Period of time and under such conditions that cell growth is maximized” and the like, refer to those culture conditions that, for a particular cell line, are determined to be optimal for cell growth and division. In some aspects, during the growth phase, cells are cultured in nutrient medium containing the necessary additives generally at about 25°-40° C., in a humidified, controlled atmosphere, such that optimal growth is achieved for the particular cell line.

In some aspects, cells are maintained in the growth phase for a period of about between one and seven days, e.g., between two to six days, e.g., six days. The length of the growth phase for the particular cells can be determined without undue experimentation. For example, the length of the growth phase will be the period of time sufficient to allow the particular cells to reproduce to a viable cell density within a range of about 20%-80% of the maximal possible viable cell density if the culture was maintained under the growth conditions. In some aspects, “maximum growth rate” refers to the growth rate of the specific cell line/clone measured during its exponential growth phase, while the cells are in fresh culture medium (e.g., measured at a time during culture when nutrients are sufficient and there is not any significant inhibition of growth from any components of the culture).

As used herein, the term “cell viability” refers to the ability of cells in culture to survive under a given set of culture conditions or experimental variations. The term as used herein also refers to that portion of cells which are alive at a particular time in relation to the total number of cells, living and dead, in the culture at that time.

As used herein, the term “cell density” refers to that number of cells present in a given volume of medium.

As used herein, the term “bioreactor” or “culture vessel” refers to any vessel used for the growth of a mammalian cell culture. The bioreactor can be of any size so long as it is useful for the culturing of mammalian cells.

As used herein, the term “bioreactor run” can include one or more of the lag phase, log phase, or plateau phase growth periods during a cell culture cycle.

As used herein, the terms “N-1 culture vessel,” “N-1 seed-train culture vessel,” “N-1 vessel,” “N1-culture,” or “N1 container” refer to a culture vessel that is immediately before the N culture vessel (production culture vessel) and is used to grow the cell culture to a high viable cell density for subsequent inoculation into N (production) culture vessel. The cell culture to be grown in the N-1 culture vessel may be obtained after culturing the cells in several vessels prior to the N-1 culture vessel, such as N-4, N-3, and N-2 vessels.

As used herein, the terms “N culture vessel,” “production culture vessel,” “N vessel,” “N bioreactor,” or “production bioreactor” refer to the cell culture in the bioreactor after the N-1 bioreactor. The N culture is used in the production of the AAV.

As used herein, the term “seeding” or “inoculating” refers to the process of providing a cell culture to a bioreactor or another vessel. In one aspect, the cells have been propagated previously in another bioreactor or vessel. In another aspect, the cells have been frozen and thawed immediately prior to providing them to the bioreactor or vessel. The term refers to any number of cells, including a single cell.

rAAV and Methods of Producing an rAAV (or Composition Comprising an rAAV) (e.g., rAAVrh74.MHCK7.Microdystrophin)

The present disclosure provides a composition comprising a recombinant adeno-associated virus (rAAV) rAAV.MHCK7.micro-dystrophin, wherein the rAAV is produced in adherent mammalian cells, and wherein the adherent cells are cultured in an N-1 container under suspension conditions. In some aspects, the rAAV is of the serotype AAVrh.74 (e.g., rAAV.MHCK7.micro-dystrophin).

The present disclosure also provides a method of producing a recombinant adeno-associated virus (rAAV) rAAVrh74.MHCK7.microdystrophin in adherent mammalian cells by a suspension seed process, comprising: (a) culturing cells with a first growth medium comprising serum in a N-2 container; (b) removing the cells from the first medium; (c) inoculating the cells from step (b) into a second medium comprising no serum or serum at a concentration less than the first medium in a N-1 container; (d) culturing the cells in the N-1 container under suspension conditions; and (e) inoculating a third medium in a bioreactor with the cells from step (d).

In some aspects, the rAAV used in the method described herein comprises the human micro-dystrophin nucleotide sequence of SEQ ID NO:1. In some aspects, the rAAV comprises the MHCK7 promoter sequence of SEQ ID NO: 7. In some aspects, the rAAV comprises the human micro-dystrophin nucleotide sequence of SEQ ID NO:1 and the MHCK7 promoter sequence of SEQ ID NO: 7.

In some aspects, the suspension seed process further comprises: (f) transfecting the adherent cells with a transgene plasmid comprising a rAAVrh74.MHCK7.microdystrophin construct, a plasmid comprising an AAV rep gene and an AAV cap gene, and an adenovirus helper plasmid.

In some aspects, the transgene plasmid comprising a rAAVrh74.MHCK7.microdystrophin construct comprises: the nucleic acid sequence of SEQ ID NO: 9; nucleotides 55-5021 of SEQ ID NO: 3; or nucleotides 1-4977 of SEQ ID NO: 8. In some aspects, the plasmid comprising an AAV rep gene and an AAV cap gene comprises an AAV2 rep gene and an rAAVrh74 cap gene. In some aspects, the adenovirus helper plasmid comprises an adenovirus 5 E2A, E4ORF6, and a VA RNA gene.

In some aspects, the suspension seed process further comprises: (g) lysing the adherent cells. In some aspects, the adherent cells are lysed by freeze-thaw, solid shear, hypertonic and/or hypotonic lysis, liquid shear, sonication, high-pressure extrusion, detergent lysis, or combinations thereof.

In some aspects, the suspension seed process further comprises (h) purifying the rAAV by at least one column chromatography step. In some aspects, the at least one column chromatography step comprises an anion exchange chromatography, a size exclusion chromatography, or a combination thereof.

In some aspects, the suspension seed process further comprises culturing cells with the first growth medium in a N-3 container. In some aspects, the suspension seed process further comprises culturing cells with the first growth medium in a N-4 container.

In some aspects, the bioreactor is an adherent bioreactor. In some aspects, the rAAV is purified from the culture produced in the adherent bioreactor.

In some aspects, the third medium in the bioreactor comprises at least one factor that promotes cell adherence. In some aspects, the at least one factor that promotes cell adherence is selected from the group consisting of serum, FBS, fibronectin, collagen, laminin, calcium ions, proteoglycans or non-proteoglycan polysaccharides of the extracellular matrix, and combinations thereof. In some aspects, the third medium in the bioreactor comprises DMEM and 10% FBS.

In some aspects, the adherent cells are cultured under suspension conditions for about 48-72 hours.

In some aspects, the N-1 container is a suspension shake flask.

In some aspects, the adherent cells are selected from the group consisting of HeLa cells, CHO cells, HEK-293 cells, VERO cells, BHK cells, MDCK cells, MDBK cells, and COS cells. In some aspects, the adherent cells are HeLa cells or HEK-293 cells. In some aspects, the adherent cells are HEK-293 cells. In some aspects, the adherent cells are not suspension-adapted. In some aspects, culturing the cells under suspension conditions does not alter the adherent-dependency of the cells. In some aspects, the culturing does not alter the cells to create a new cell line.

In some aspects, the suspension seed process used to produce rAAVrh74.MHCK7.microdystrophin in adherent mammalian cells comprises:
(a) culturing cells with a first growth medium comprising serum in a N-2 container;
(b) removing the cells from the first medium;
(c) inoculating the cells from step (b) into a second medium comprising no serum or serum at a concentration less than the first medium in a N-1 container;
(d) culturing the cells in the N-1 container under suspension conditions;
(e) inoculating a third medium in a bioreactor with the cells from step (d),
(f) transfecting the cells with a transgene plasmid comprising rAAVrh74.MHCK7.microdystrophin construct, a plasmid comprising an AAV rep gene and an AAV cap gene, and an adenovirus helper plasmid,
(g) lysing the cells, and
(h) purifying the rAAV by at least one column chromatography step.

In some aspects, the rAAVs are described in International Publ. No. WO 2019/245973 A1, which is expressly incorporated herein by reference in its entirety.

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol. 45: 555-564 (1983) as corrected by Ruffing et al., J Gen Virol. 75: 3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol. 78: 6381-6388 (2004); the AAV-10 genome is provided in Mol. Ther., 13(1): 67-76 (2006); and the AAV-11 genome is provided in Virology, 330(2): 375-383 (2004). Cloning of the AAVrh.74 serotype is described in Rodino-Klapac et al., Journal of Translational Medicine 5: 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther 8: 659-669 (1997); Kessler et al., Proc Nat. Acad Sc. USA 93: 14082-14087 (1996); and Xiao et al., J Virol 70: 8098-8108 (1996). See also, Chao et al., Mol Ther 2:619-623 (2000) and Chao et al., Mol Ther 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA 94: 5804-5809 (1997) and Murphy et al., Proc Natl Acad Sci USA 94: 13921-13926 (1997). Moreover, Lewis et al., J Virol. 76: 8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the present disclosure comprise nucleic acid molecule of the present disclosure and one or more AAV ITRs flanking a nucleic acid molecule. AAV DNA in the rAAV genomes may be from any AAV serotype for which a recombinant virus can be derived including, but not limited to, AAV serotypes AAVrh.74, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy 22(11): 1900-1909 (2014). As noted in the Background section above, the nucleotide sequences of the genomes of various AAV serotypes are known in the art. To promote skeletal muscle specific expression, AAV1, AAV6, AAV8 or AAVrh.74 can be used.

DNA plasmids of the present disclosure comprise rAAV genomes of the present disclosure. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., Proc. Natl. Acad. S6. USA 79:2077-2081 (1982), addition of synthetic linkers containing restriction endonuclease cleavage sites (Laughlin et al., Gene 23:65-73 (1983)) or by direct, blunt-end ligation (Senapathy & Carter, J. Biol. Chem. 259:4661-4666 (1984)). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, Current Opinions in Biotechnology 1533-539 (1992); and Muzyczka, N., Curr. Topics Microbial. Immunol. 158: 97-129 (1992). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The present disclosure thus provides packaging cells that produce infectious rAAV. In one aspect packaging cells may be stably transformed cancer cells such as HeLa cells, 293 cells, and PerC.6 cells (a cognate 293 line). In another aspect, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the present disclosure comprise a rAAV genome. In exemplary aspects, the genomes of both rAAV lack AAV rep and cap DNA, that is, there is no AAV rep or cap DNA between the ITRs of the genomes. Examples of rAAV that may be constructed to comprise the nucleic acid molecules of the present disclosure are set out in International Patent Application No. PCT/US2012/047999 (WO 2013/016352) incorporated by reference herein in its entirety.

In an exemplary aspect, the recombinant AAV vector of the invention is produced by the triple transfection method (Xiao et al., J Virol 72: 2224-2232 (1998)) using the AAV vector plasmids rAAV.MHCK7.micro-dystrophin, pNLRep2-Caprh74, and pHELP. The rAAV contains the micro-dystrophin gene expression cassette flanked by AAV2 inverted terminal repeat sequences (ITR). It is this sequence that is encapsidated into AAVrh74 virions. The plasmid contains the micro-dystrophin sequence and the MHCK7 enhancer and core promoter elements of the muscle specific promoter to drive gene expression. The expression cassette also contains an SV40 intron (SD/SA) to promote high-level gene expression and the bovine growth hormone polyadenylation signal is used for efficient transcription termination.

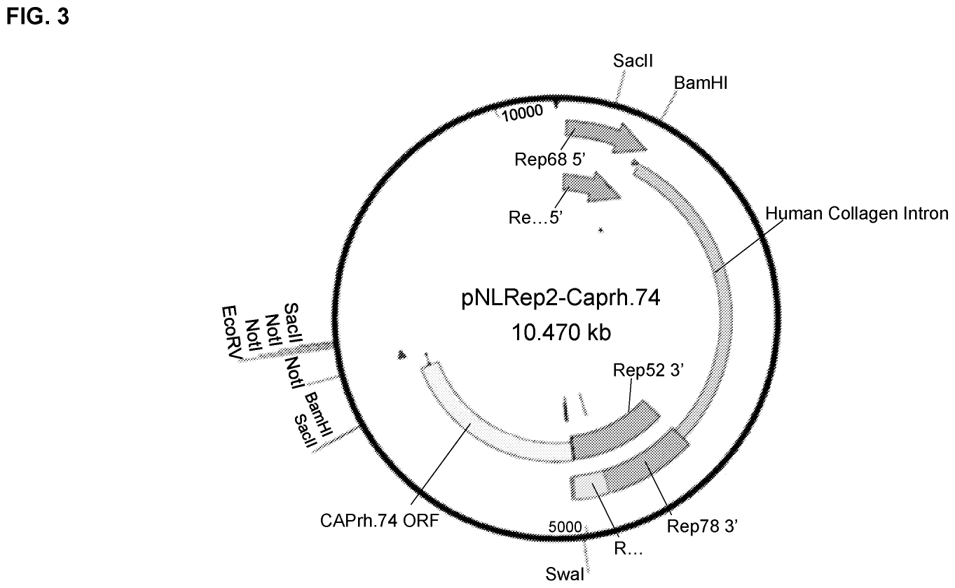

The pNLREP2-Caprh74 is an AAV helper plasmid that encodes the 4 wild-type AAV2 rep proteins and the 3 wild-type AAV VP capsid proteins from serotype rh74. A schematic map of the pNLREP2-Caprh74 plasmid is shown in FIG. 3.

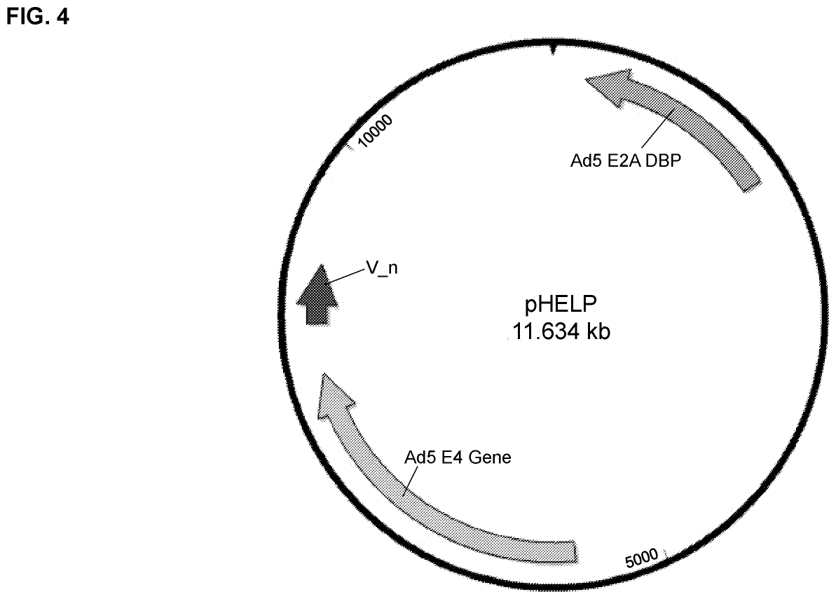

The pHELP adenovirus helper plasmid is 11,635 bp and was obtained from Applied Viromics. The plasmid contains the regions of adenovirus genome that are important for AAV replication, namely E2A, E4ORF6, and VA RNA (the adenovirus E1 functions are provided by the 293 cells). The adenovirus sequences present in this plasmid only represents ˜40% of the adenovirus genome, and does not contain the cis elements critical for replication such as the adenovirus terminal repeats. Therefore, no infectious adenovirus is expected to be generated from such a production system. A schematic map of the pHELP plasmid is shown in FIG. 4.

The rAAV, as disclosed herein, may be purified by methods standard in the art such as by column chromatography or cesium chloride gradients. Methods for purifying rAAV vectors from helper virus are known in the art and include methods disclosed in, for example, Clark et al., Hum. Gene Ther. 10(6): 1031-1039 (1999); Schenpp and Clark, Methods Mol. Med. 69 427-443 (2002); U.S. Pat. No. 6,566,118 and WO 98/09657.

In one aspect, the present disclosure provides for a rAAV comprising a muscle specific control element nucleotide sequence, and a nucleotide sequence encoding the micro-dystrophin protein. For example, the nucleotide sequence encodes a functional micro-dystrophin protein, wherein the nucleotide has, e.g., at least 65%, at least 70%, at least 75%, at least 80%, 81%, 82%, 83%, 84%, 85%, 86%, 87%, 88%, or 89% more typically at least 90%, 91%, 92%, 93%, or 94% and even more typically at least 95%, 96%, 97%, 98%, 99%, or 100% sequence identity to SEQ ID NO: 1, wherein the protein retains micro-dystrophin activity. The micro-dystrophin protein provides stability to the muscle membrane during muscle contraction, e.g., micro-dystrophin acts as a shock absorber during muscle contraction.

In one aspect, the rAAV is rAAVrh74.MHCK7.microdystrophin, i.e., the viral particle form of the rAAV serotype rh74 capsid encapsidating a nucleic acid expression cassette or genome that comprises the microdystrophin transgene driven by an MHCK7 promoter/enhancer, and also referred to by the non-proprietary drug name delandistrogene moxeparvovec in the context of administration to a subject. In some aspects, when referring to study subjects in the Examples that were administered delandistrogene moxeparvovec, the data (e.g., bar graphs) may be designated more simply as “Treated.”

In one aspect, the rAAVrh74.MHCK7.microdystrophin is the rAAVrh74.MHCK7.microdystrophin of SEQ ID NO: 9 or of nucleotides 55-5021 of SEQ ID NO: 3, nucleotides 1-4977 of SEQ ID NO: 8, or nucleotides 56-5022 of SEQ ID NO: 6, wherein the rAAV is produced in adherent cells, and wherein the adherent cells are cultured in an N-1 container under suspension conditions. In one aspect, the rAAV is AAVrh74.MCK.microdystrophin. In one aspect, the rAAVrh74.MCK.microdystrophin is the rAAVrh74.MCK.microdystrophin of nucleotides 56-4820 of SEQ ID NO: 5.

The present disclosure also provides for rAAV wherein the nucleotide sequence comprises a nucleotide sequence that hybridizes under stringent conditions to the nucleic acid sequence of SEQ ID NO: 1, or compliments thereof, and encodes a functional micro-dystrophin protein.

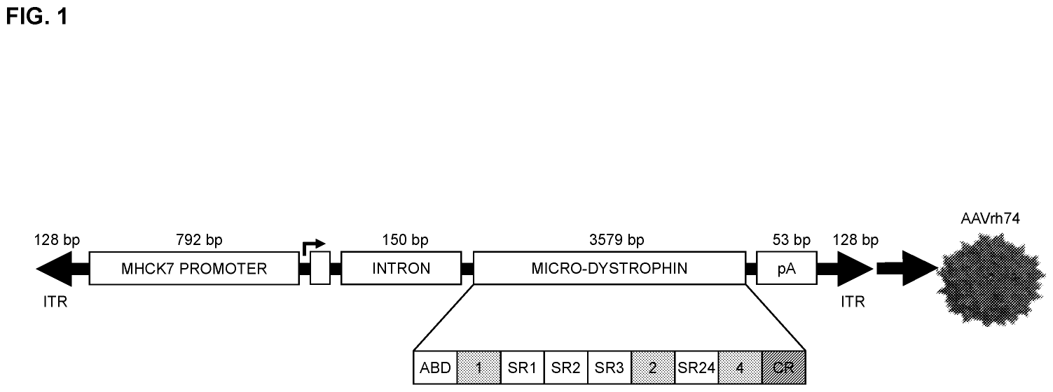

In one aspect, the rAAV is a non-replicating, recombinant adeno-associated virus (AAV) termed rAAVrh74.MHCK7.micro-dystrophin of SEQ ID NO: 9, nucleotides 55-5021 of SEQ ID NO: 3, nucleotides 1-4977 of SEQ ID NO: 8, or nucleotides 56-5022 of SEQ ID NO: 6, wherein the rAAV is produced in adherent cells, and wherein the adherent cells are cultured in an N-1 container under suspension conditions. This vector genome contains minimal elements required for gene expression, including AAV2 inverted terminal repeats (ITR), the micro-dystrophin, SV40 intron (SD/SA), and synthetic polyadenylation (Poly A) signal, all under the control of the MHCK7 promoter/enhancer. The schematic of the vector genome and expression cassette is shown in FIG. 1. The AAVrh74 serotype can be employed to achieve efficient gene transfer in skeletal and cardiac muscle following IV administration.

In one aspect, the present disclosure provides for a rAAV wherein the muscle specific control element is a human skeletal actin gene element, cardiac actin gene element, myocyte-specific enhancer binding factor (MEF), muscle creatine kinase (MCK), truncated MCK (tMCK), myosin heavy chain (MHC), hybrid α-myosin heavy chain enhancer-/MCK enhancer-promoter (MHCK7), C5-12, murine creatine kinase enhancer element, skeletal fast-twitch troponin c gene element, slow-twitch cardiac troponin c gene element, the slow-twitch troponin i gene element, hypoxia-inducible nuclear factors, steroid-inducible element or glucocorticoid response element (GRE).

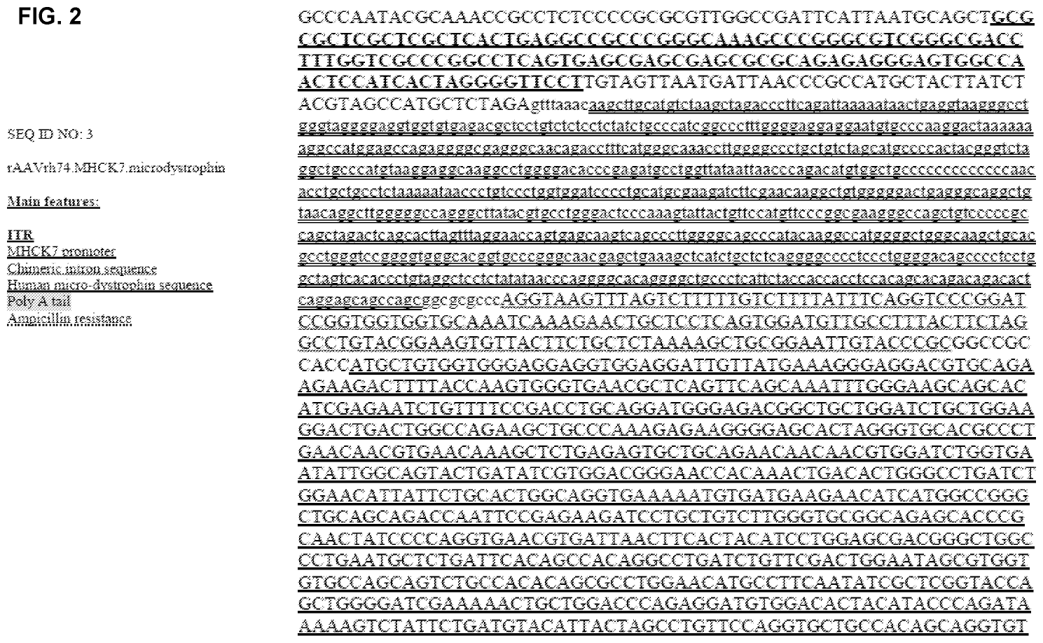

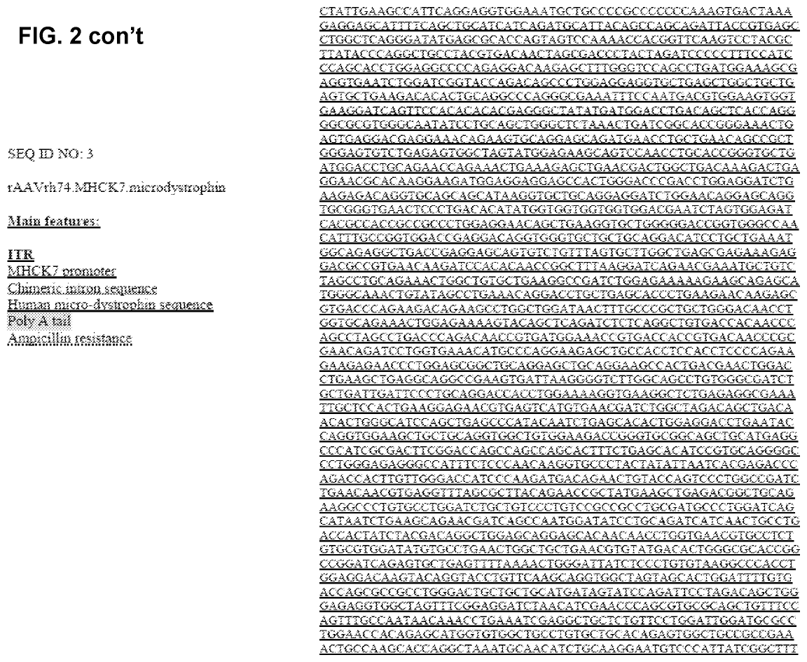

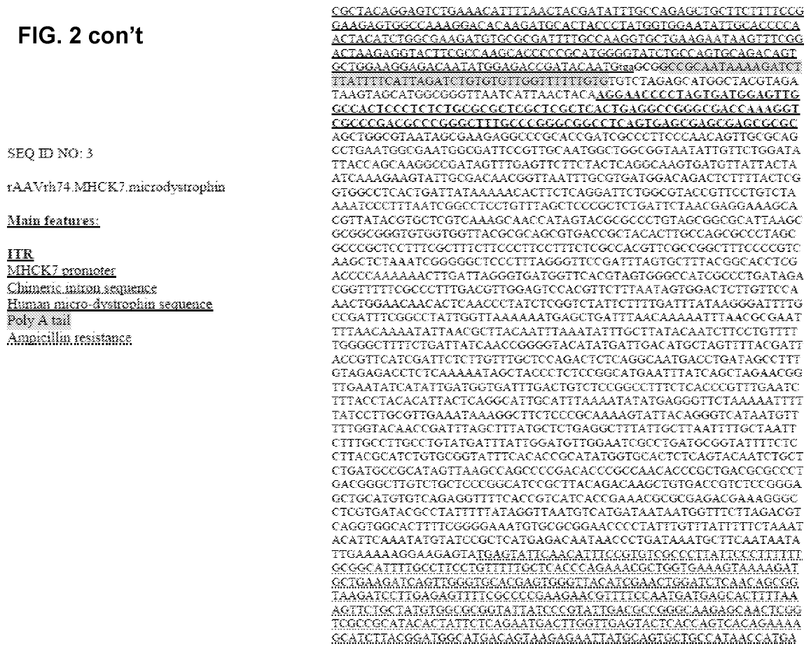

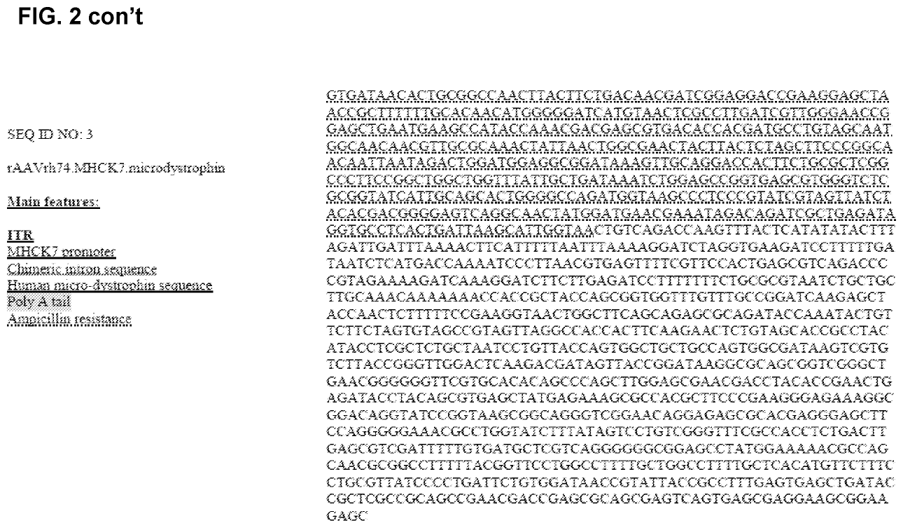

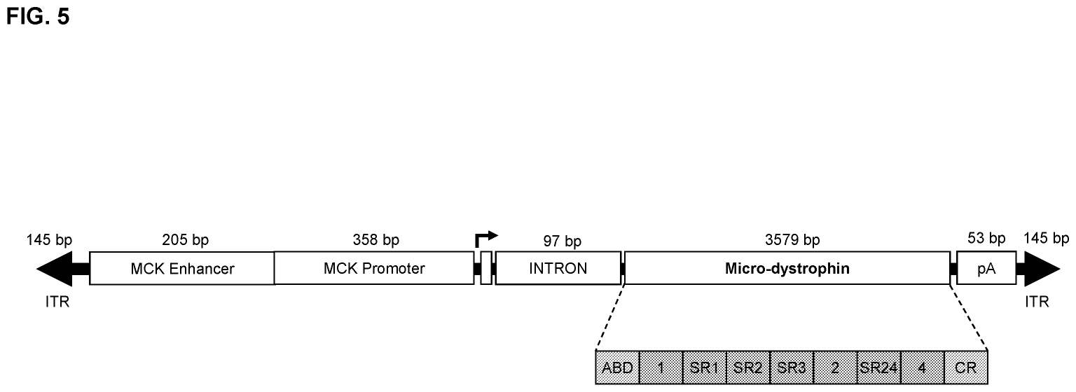

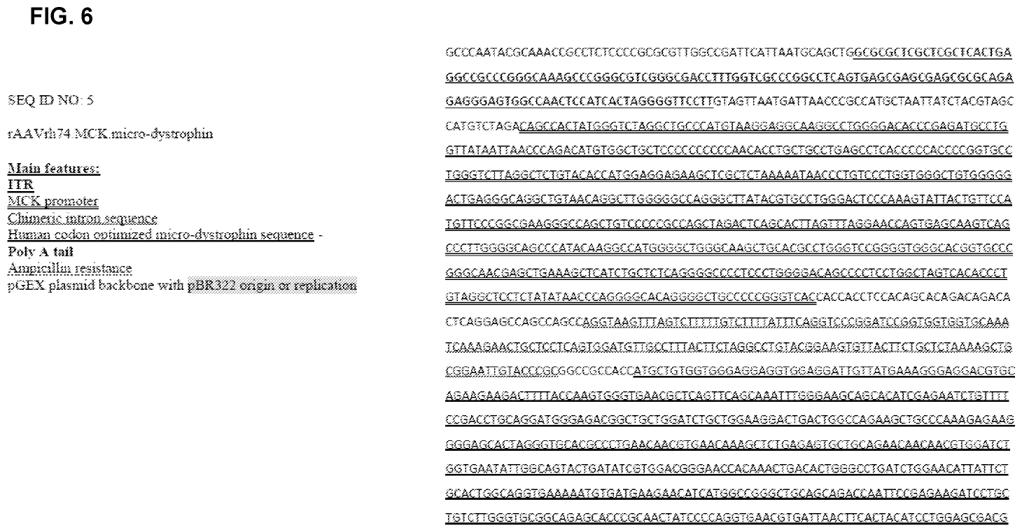

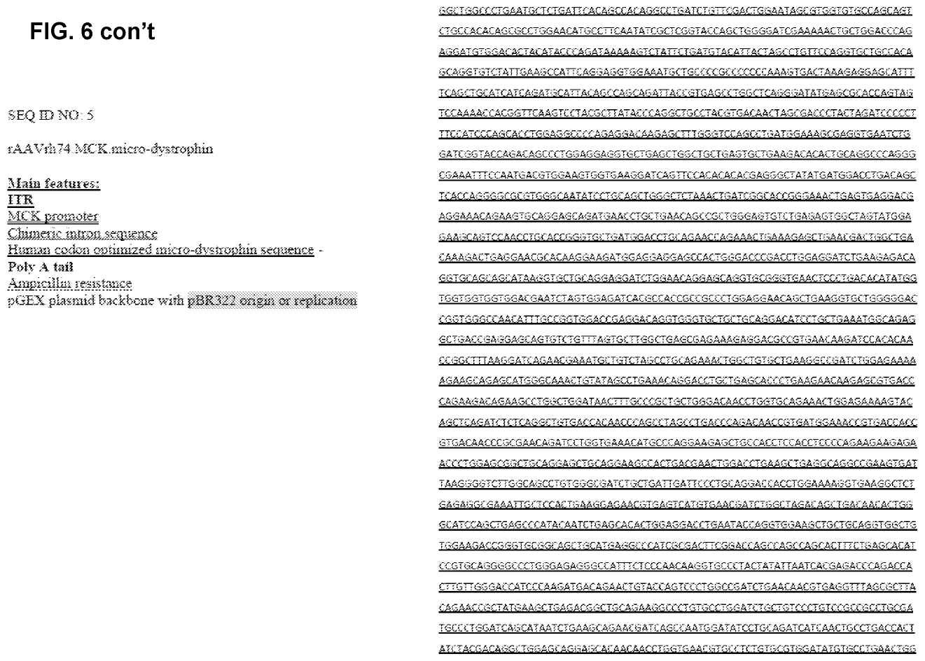

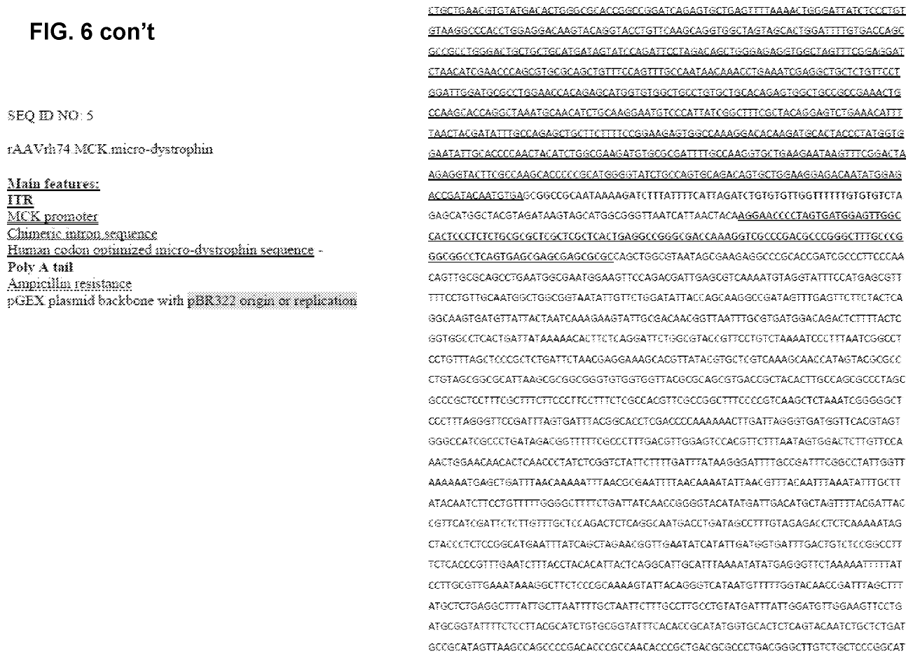

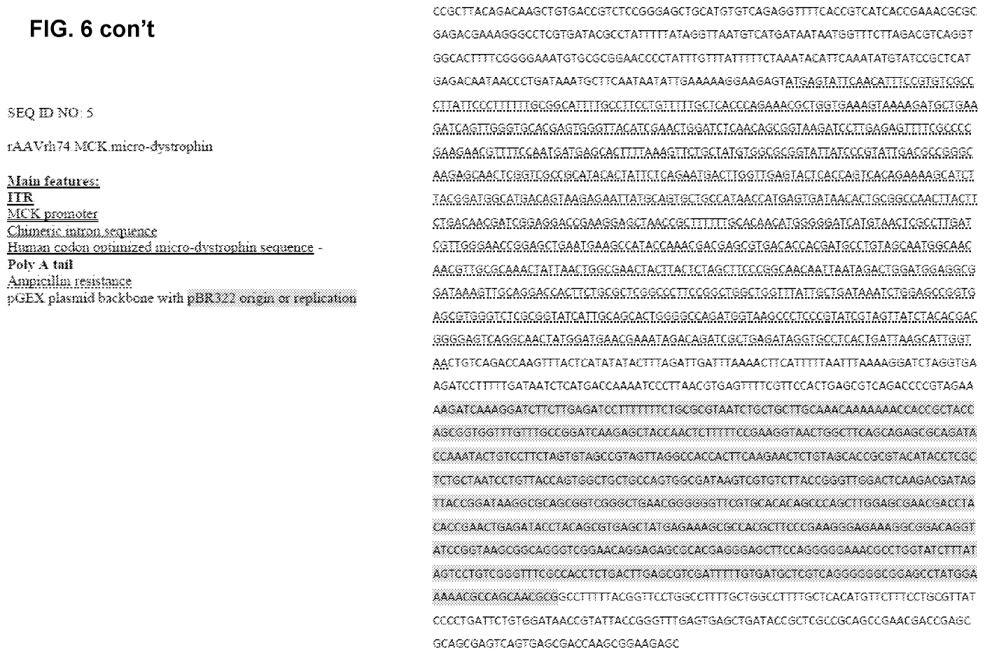

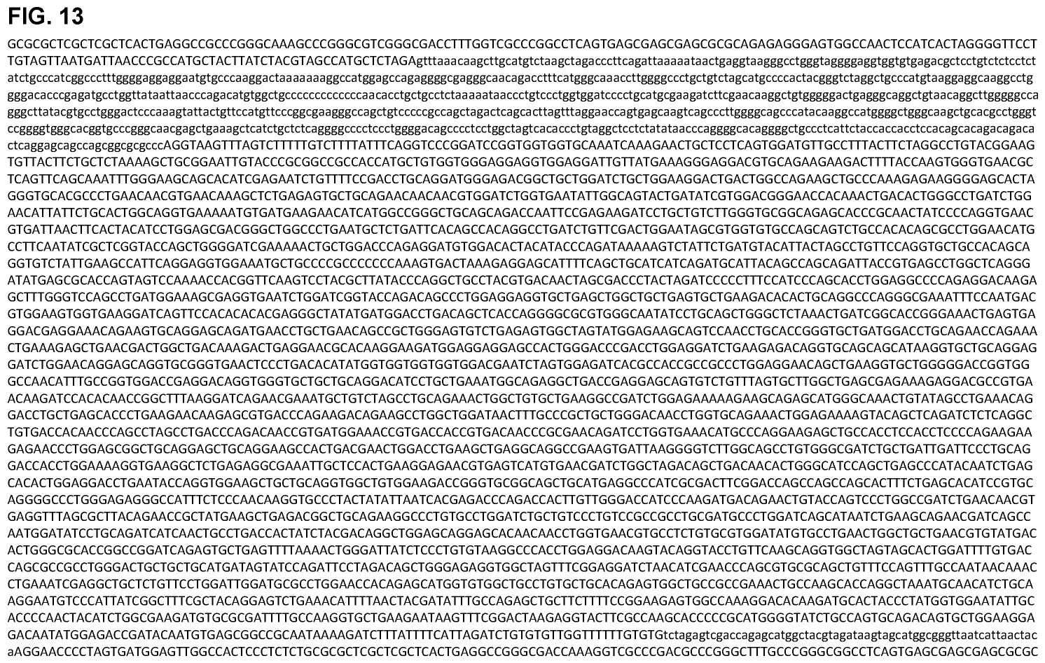

For example, the muscle specific control element is the MHCK7 promoter nucleotide sequence SEQ ID NO: 2 or SEQ ID NO: 7, or the muscle specific control element is MCK nucleotide sequence SEQ ID NO: 4. In addition, in any of the rAAV vectors of the present disclosure, the muscle specific control element nucleotide sequence, e.g. MHCK7 or MCK nucleotide sequence, is operably linked to the nucleotide sequence encoding the micro-dystrophin protein. For example, the MHCK7 promoter nucleotide sequence (SEQ ID NO: 2 or SEQ ID NO: 7) is operably linked to the human micro-dystrophin coding sequence (SEQ ID NO: 1) as set out in the construct provided in FIG. 1 or FIG. 2 (SEQ ID NO: 3) or FIG. 13 (SEQ ID NO: 9). In another example, the MCK promoter (SEQ ID NO: 4) is operably linked to the human micro-dystrophin coding sequence (SEQ ID NO: 1) as set out in the construct provided in FIG. 5 or FIG. 6 (SEQ ID NO: 5). In another aspect, the present disclosure provides for a rAAV vector comprising the nucleotide sequence of SEQ ID NO: 1 and SEQ ID NO: 2, or SEQ ID NO: 1 and SEQ ID NO: 7. The present disclosure also provides for a rAAV vector comprising the nucleotide sequence of SEQ ID NO: 1 and SEQ ID NO: 4.

In a further aspect, the present disclosure provides for an rAAV construct contained in the plasmid comprising the nucleotide sequence of SEQ ID NO: 3, SEQ ID NO: 5, SEQ ID NO: 6, or SEQ ID NO: 8. For example, the AAVrh74.MHCK7.microdystrophin vector comprises the nucleotide sequence within and inclusive of the ITRs of SEQ ID NO: 3 and shown in FIG. 2. In another aspect, the rAAV vector comprises the 5′ ITR, MHCK7 promoter, a chimeric intron sequence, the coding sequence for the human micro-dystrophin gene, polyA, and 3′ ITR. In one aspect, the vector comprises nucleotides 55-5021 of SEQ ID NO:3. The plasmid set forth in SEQ ID NO:3 further comprises ampicillin resistance and the pGEX plasmid backbone with pBR322 origin of replication.

In another aspect, the present disclosure provides for a rAAV comprising the nucleotide sequence of SEQ ID NO: 9, wherein the rAAV is produced in adherent cells, and wherein the adherent cells are cultured in an N-1 container under suspension conditions. For example, the AAVrh74.MHCK7.microdystrophin vector construct comprises the nucleotide sequence of SEQ ID NO: 9 and shown in FIG. 13. This rAAV vector construct comprises the MHCK7 promoter, a chimeric intron sequence, the coding sequence for the human micro-dystrophin gene, and polyA. In one aspect, the rAAV vector construct further comprises an ITR 5′ to the promoter, and an ITR 3′ to the polyA. In one aspect, the rAAV is AAVrh74.

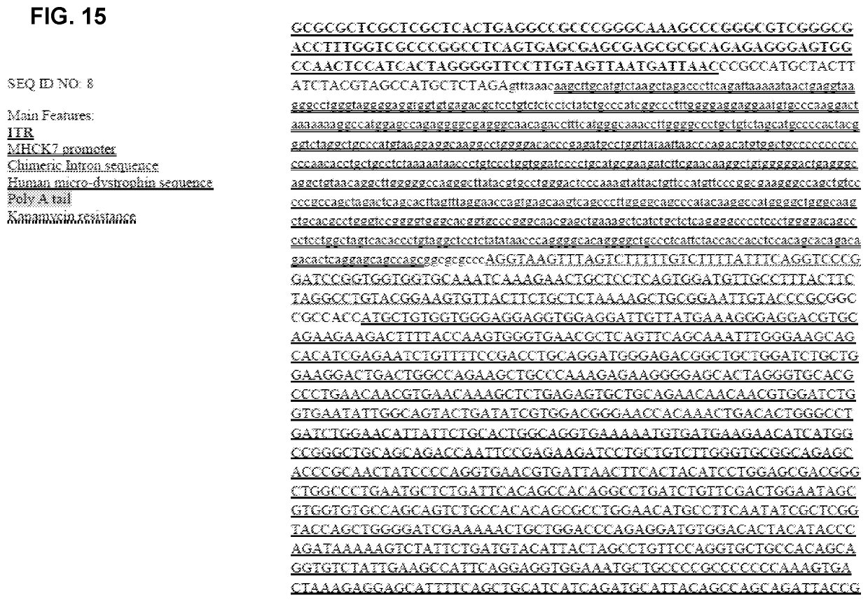

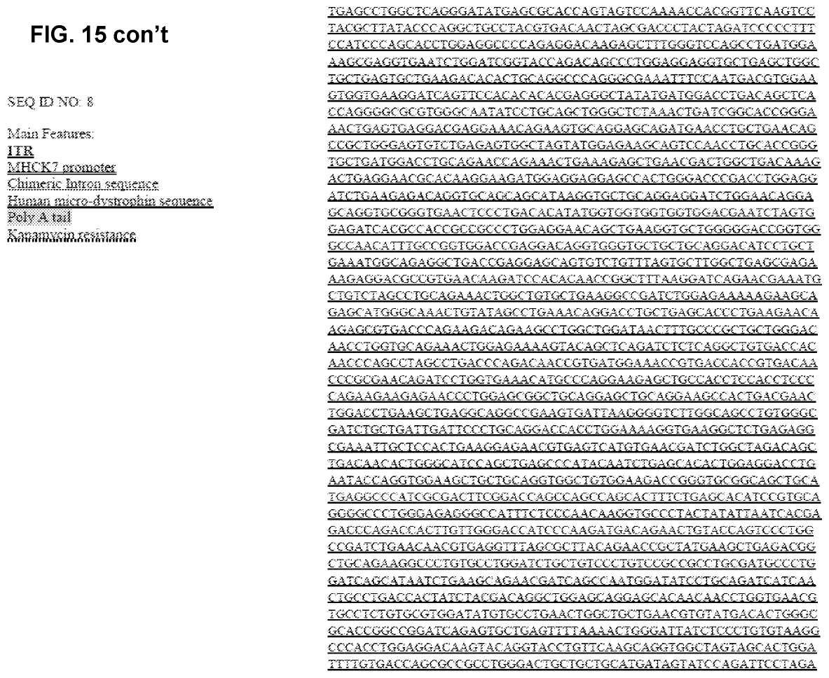

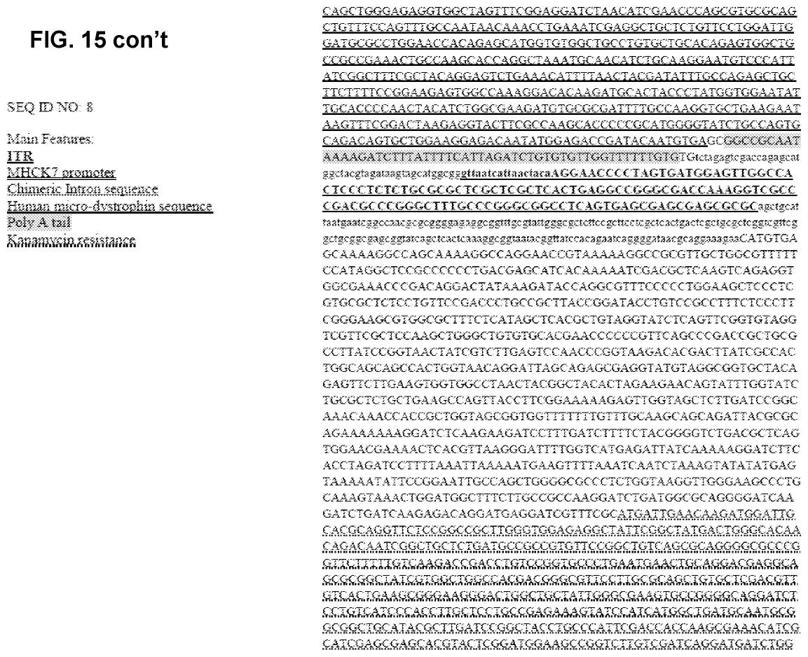

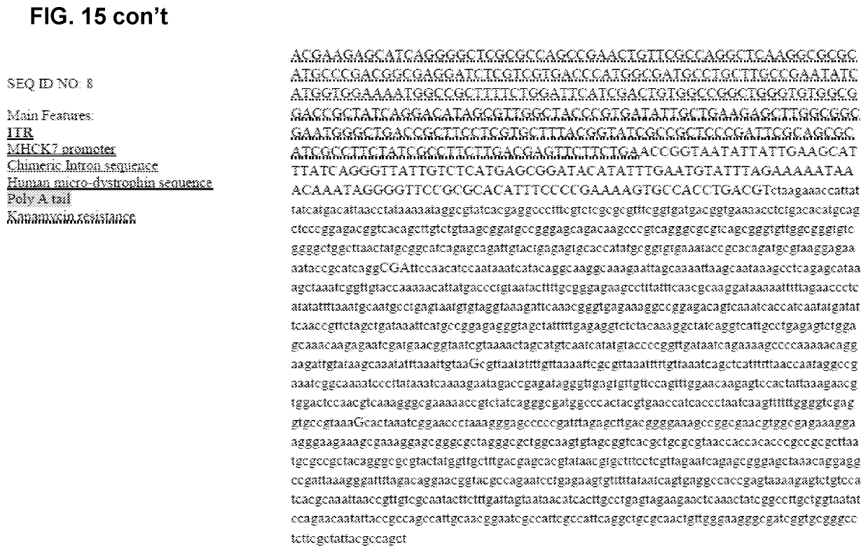

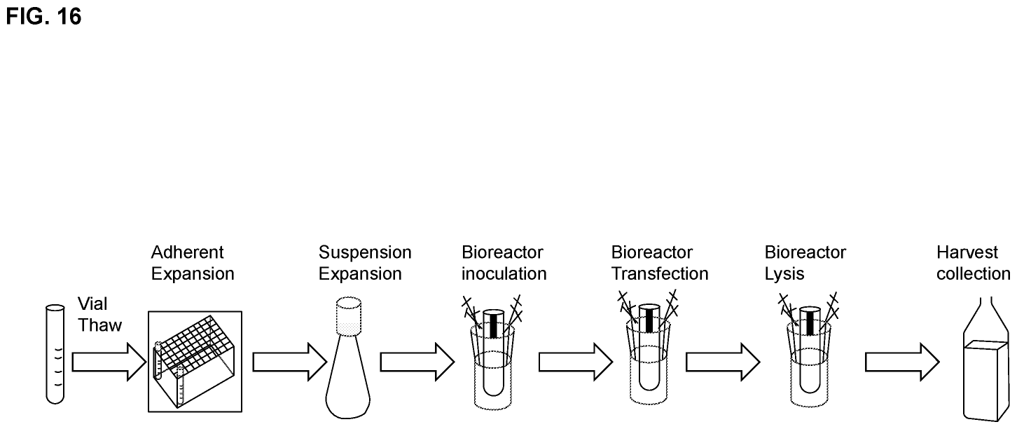

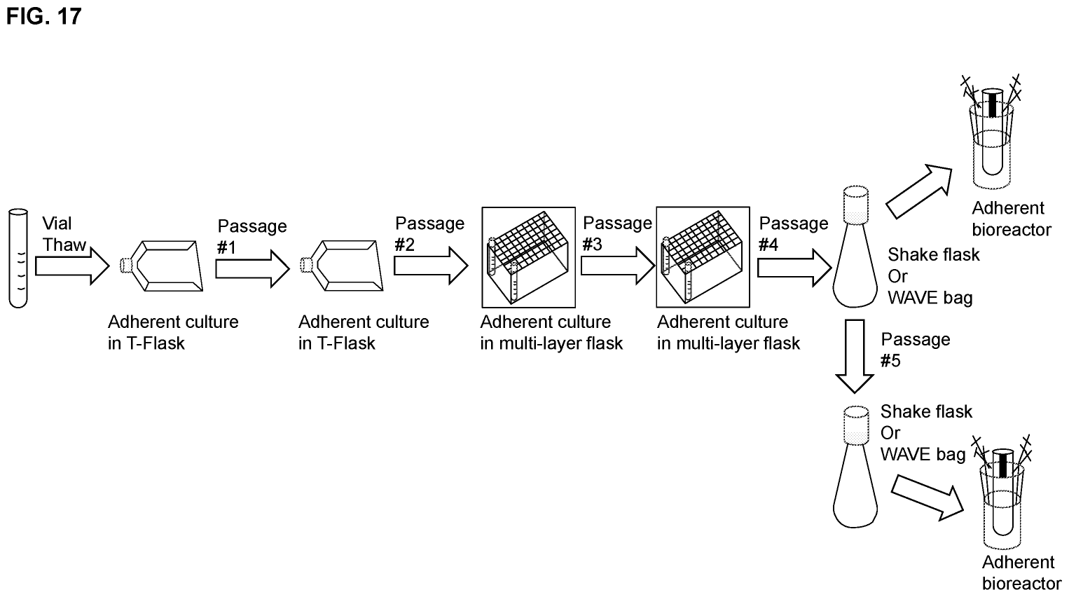

In another aspect, the rAAVrh74.MHCK7.microdystrophin vector (i.e. viral vector) comprises the nucleotide sequence within and inclusive of the ITRs of SEQ ID NO: 8 and shown in FIG. 15. The rAAV vector comprises the 5′ ITR, MHCK7 promoter, a chimeric intron sequence, the coding sequence for the human micro-dystrophin gene, polyA, and 3′ ITR. In one aspect, the vector comprises nucleotides 1-4977 of SEQ ID NO:9. The plasmid set forth in SEQ ID NO:3 further comprises kanamycin resistance and the pGEX plasmid backbone with pBR322 origin of replication.

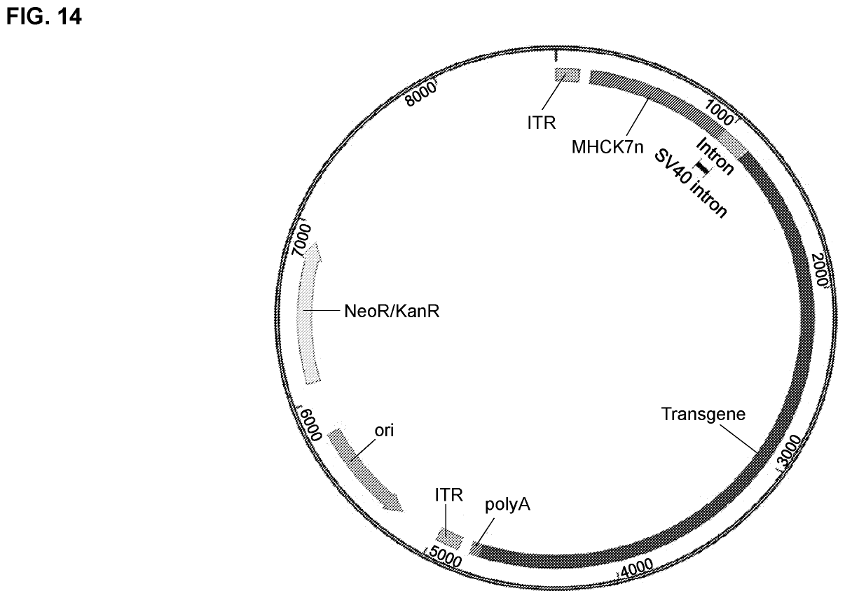

In another aspect, the present disclosure provides for a plasmid comprising the AAVrh74.MHCK7.microdystrophin construct. In one aspect, the plasmid comprises the 5′ ITR, MHCK7 promoter, a chimeric intron sequence, the coding sequence for the human micro-dystrophin gene, polyA, and 3′ ITR. In one aspect, the plasmid comprises kanamycin resistance and optionally comprises the pGEX plasmid backbone with pBR322 origin of replication. In a particular aspect, the plasmid is set forth in SEQ ID NO:8, and shown in FIGS. 14 and 15.

The present disclosure provides for a recombinant AAV vector comprising the human micro-dystrophin nucleotide sequence of SEQ ID NO: 1 and the MHCK7 promoter nucleotide sequence of SEQ ID NO: 2 or SEQ ID NO:7, wherein the rAAV is produced in adherent cells, and wherein the adherent cells are cultured in an N-1 container under suspension conditions. This rAAV vector is the AAV serotype AAVrh.74.

The present disclosure also provides for a rAAV comprising the AAVrh74.MHCK7.micro-dystrophin construct nucleotide sequence within and inclusive of the ITR's in SEQ ID NO: 3, the nucleotide sequence within and inclusive of the ITR's in SEQ ID NO: 8 or the nucleotide sequence as set forth in SEQ ID NO: 9, wherein the rAAV is produced in adherent cells, and wherein the adherent cells are cultured in an N-1 container under suspension conditions. This rAAV vector is the AAV serotype AAVrh.74.

The rAAV vectors of the present disclosure may be any AAV serotype, such as the serotype AAVrh.74, AAV1, AAV2, AAV4, AAV5, AAV6, AAV7, AAV8, AAV9, AAV10, AAV11, AAV12 or AAV13.

The present disclosure also provides for pharmaceutical compositions (or sometimes referred to herein as simply “compositions”) comprising any of the rAAV vectors of the present disclosure.

In another aspect, the present disclosure provides for methods of producing a rAAV vector particle comprising culturing a cell that has been transfected with any rAAV vector of the present disclosure and recovering rAAV particles from the supernatant of the transfected cells. The present disclosure also provides for viral particles comprising any of the recombinant AAV vectors of the present disclosure.

Compositions Comprising rAAV and its Administration

In another aspect, the present disclosure contemplates compositions comprising the rAAV of the present disclosure. Compositions of the present disclosure comprise rAAV and a pharmaceutically acceptable carrier. The compositions may also comprise other ingredients such as diluents and adjuvants. Acceptable carriers, diluents, and adjuvants are nontoxic to recipients and are preferably inert at the dosages and concentrations employed and include buffers and surfactants such as pluronics.

The present disclosure provides a composition for treating muscular dystrophy (e.g., DMD) in a subject in need thereof comprising a recombinant adeno-associated virus (rAAV) rAAVrh74.MHCK7.micro-dystrophin, wherein the rAAV is produced in adherent cells, and wherein the adherent cells are cultured in an N-1 container under suspension conditions.

In some aspects, the present disclosure provides a composition comprising a recombinant adeno-associated virus (rAAV) rAAVrh74.MHCK7.microdystrophin, wherein the rAAV is produced in adherent mammalian cells by the suspension seed process described herein. In some aspects, the composition comprises: a) rAAV particles comprising the nucleic acid sequence of SEQ ID NO: 9; b) rAAV particles comprising nucleotides 55-5021 of SEQ ID NO: 3; and/or c) rAAV particles comprising nucleotides 1-4977 of SEQ ID NO: 8.

Titers of rAAV to be administered in the methods of the present disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). One exemplary method of determining encapsilated vector genome titer uses quantitative PCR such as the methods described in (Pozsgai et al., Mol. Ther. 25: 855-869 (2017)).

Methods of transducing a target cell with rAAV, in vivo or in vitro, are contemplated by the present disclosure. The in vivo methods comprise the step of administering an effective dose, or effective multiple doses, of a composition comprising a rAAV of the present disclosure to an animal (including a human being) in need thereof. If the dose is administered prior to development of a disorder/disease, the administration is prophylactic. If the dose is administered after the development of a disorder/disease, the administration is therapeutic. In aspects of the present disclosure, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the present disclosure is DMD.

Combination therapies are also contemplated by the present disclosure. Combination as used herein includes both simultaneous treatment and sequential treatments. Combinations of methods of the present disclosure with standard medical treatments (e.g., corticosteroids) are specifically contemplated, as are combinations with novel therapies.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the present disclosure may be chosen and/or matched by those skilled in the art taking into account the infection and/or disease state being treated and the target cells/tissue(s) that are to express the micro-dystrophin protein.

The present disclosure provides for local administration and systemic administration of an effective dose of rAAV and compositions of the present disclosure. For example, systemic administration is administration into the circulatory system so that the entire body is affected. Systemic administration includes enteral administration such as absorption through the gastrointestinal tract and parenteral administration through injection, infusion or implantation.

In particular, actual administration of rAAV of the present disclosure may be accomplished by using any physical method that will transport the rAAV recombinant vector into the target tissue of an animal. Administration according to the present disclosure includes, but is not limited to, injection into muscle and injection into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the present disclosure. The rAAV can be used with any pharmaceutically acceptable carrier for ease of administration and handling.

In one aspect of the present disclosure, the AAVrh74.MHCK7.microdystrophin described herein is formulated in a buffer containing 20 mM Tris (pH 8.0), 1 mM magnesium chloride (MgCl₂), 200 mM sodium chloride (NaCl), and 0.001% poloxamer 188.

The dose of rAAV to be administered in methods disclosed herein will vary depending, for example, on the particular rAAV, the mode of administration, the treatment goal, the individual, and the cell type(s) battractiveing targeted, and may be determined by methods standard in the art. Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 2×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 2×10¹⁴ vg, 1×10¹⁵ vg respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1.25×10¹⁴ vg/kg, 1.5×10¹⁴ vg/kg, 1.75×10¹⁴ vg/kg, 2.0×10¹⁴ vg/kg, 2.25×10¹⁴ vg/kg, 2.5×10¹⁴ vg/kg, 2.75×10¹⁴ vg/kg, 3.0×10¹⁴ vg/kg, 3.25×10¹⁴ vg/kg, 3.5×10¹⁴ vg/kg, 3.75×10¹⁴ vg/kg, 4.0×10¹⁴ vg/kg, 1×10¹⁵ vg/kg respectively). Methods for titering AAV are described in Clark et al., Hum. Gene Ther., 10: 1031-1039 (1999).

In particular, actual administration of rAAV of the present disclosure may be accomplished by using any physical method that will transport the rAAV recombinant vector into the target tissue of an animal. Administration according to the present disclosure includes, but is not limited to, injection into muscle and injected into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the present disclosure. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one aspect, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one aspect, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the present disclosure results in sustained expression of the micro-dystrophin protein. The present disclosure thus provides methods of administering/delivering rAAV which express micro-dystrophin protein to an animal, preferably a human being. These methods include transducing tissues (including, but not limited to, tissues such as muscle, organs such as liver and brain, and glands such as salivary glands) with one or more rAAV of the present disclosure. Transduction may be carried out with gene cassettes comprising tissue specific control elements. For example, one aspect of the present disclosure provides methods of transducing muscle cells and muscle tissues directed by muscle specific control elements, including, but not limited to, those derived from the actin and myosin gene families, such as from the myoD gene family (See Weintraub et al., Science, 251: 761-766 (1991)), the myocyte-specific enhancer binding factor MEF-2 (Cserjesi and Olson, Mol Cell Biol 11: 4854-4862 (1991)), control elements derived from the human skeletal actin gene (Muscat et al., Mol Cell Biol, 7: 4089-4099 (1987)), the cardiac actin gene, muscle creatine kinase sequence elements (See Johnson et al., Mol CellBiol 9:3393-3399 (1989)) and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

Muscle tissue is an attractive target for in vivo DNA delivery because it is not a vital organ and is easy to access. The present disclosure contemplates sustained expression of micro-dystrophin from transduced myofibers.

Thus, the present disclosure provides methods of administering an effective dose (or doses, administered essentially simultaneously or doses given at intervals) of rAAV that encode micro-dystrophin to a subject in need thereof (e.g., a subject with muscular dystrophy).

The present disclosure provides for nucleic acid molecules comprising the nucleotide sequence of SEQ ID NO: 3, 8, or 9. The present disclosure also provides for rAAV comprising the nucleic acid sequence of SEQ ID NO: 9 or nucleotides 1-4977 of SEQ ID NO: 8 or nucleotides 55-5021 of SEQ ID NO: 3, and rAAV particles comprising the nucleic acid sequence of SEQ ID NO: 9 or nucleotides 1-4977 of SEQ ID NO: 8 or nucleotides 55-5021 of SEQ ID NO: 3, wherein the rAAV is produced in adherent cells, and wherein the adherent cells are cultured in an N-1 container under suspension conditions.

Another aspect of the present disclosure provides for compositions comprising a nucleic acid molecule comprising the nucleotide sequence of SEQ ID NO: 3, 8, or 9, rAAV comprising the nucleic acid sequence of SEQ ID NO: 9 or nucleotides 1-4977 of SEQ ID NO: 8 or nucleotides 55-5021 of SEQ ID NO: 3, and rAAV particles comprising the nucleic acid sequence of SEQ ID NO: 9 or nucleotides 1-4977 of SEQ ID NO: 8 or nucleotides 55-5021 of SEQ ID NO: 3, wherein the rAAV is produced in adherent cells, and wherein the adherent cells are cultured in an N-1 container under suspension conditions. Any of the methods disclosed herein may be carried out with these compositions.

### Hybrid Seed Train Expansion of Adherent Cells

Some aspects of the disclosure are related to a method of cell expansion comprising: (a) culturing cells with a first medium comprising serum in a N-2 container; (b) removing the cells from the first medium; (c) inoculating the cells from step (b) into a second medium comprising no serum or serum at a concentration less than the first medium in a N-1 container; (d) culturing the cells in the N-1 container under suspension conditions; and (e) inoculating a third medium in a bioreactor with the cells from step (d). In one aspect, the second medium is a serum-free medium. In another aspect, the second medium comprises the serum at a concentration less than the serum concentration in the first medium.

Some aspects of the disclosure are related to a method of seed-train expansion comprising (a) culturing cells with a first medium comprising serum in a N-3 container; (b) removing the cells from the first medium; (c) inoculating the cells from step (b) into a second medium comprising no serum or serum at a concentration less than the first medium in a N-2 container; (d) culturing the cells in the N-2 container under suspension conditions; (e) and inoculating the cells from step (d) into the second medium in a N-1 vessel; and (f) inoculating a third medium in a bioreactor with the cells from step (d). In one aspect, the second medium is a serum-free medium. In another aspect, the second medium comprises the serum at a concentration less than the serum concentration in the first medium.

Some aspects of the disclosure are related to a method of cell expansion of adherent cells comprising (a) culturing the adherent cells under adherent conditions in a first medium comprising serum; (b) removing the adherent cells from the first medium; (c) suspending the adherent cells in a second medium comprising no serum or serum at a concentration less than the first medium; (d) culturing the adherent cells under suspension conditions; and (e) inoculating a third medium in a bioreactor with the adherent cells from step (d). In some aspects, the method can further comprise passaging the adherent cells of step (a) at least once under adherent conditions. In some aspects, the method can further comprise further comprising passaging the adherent cells of step (d) at least one time under suspension conditions. In one aspect, the second medium is a serum-free medium. In another aspect, the second medium comprises the serum at a concentration less than the first medium in a N-1 container.

The first medium, second medium, and third medium can be any medium suitable for the particular cell being cultured. In some aspects, the medium contains e.g., inorganic salts, carbohydrates (e.g., sugars such as glucose, galactose, maltose or fructose), amino acids, vitamins (e.g., B group vitamins (e.g., B 12), vitamin A, vitamin E, riboflavin, thiamine and biotin), fatty acids and lipids (e.g., cholesterol and steroids), proteins and peptides (e.g., albumin, transferrin, fibronectin, and fetuin), serum (e.g., compositions comprising albumins, growth factors and growth inhibitors, such as, fetal bovine serum, newborn calf serum and horse serum), trace elements (e.g., zinc, copper, selenium and tricarboxylic acid intermediates), hydrolysates (hydrolyzed proteins derived from plant or animal sources), and combinations thereof. The growth medium can be a commercially available media such as 5×-concentrated DMEM/F12 (Invitrogen), CD OptiCHO feed (Invitrogen), CD EfficientFeed (Invitrogen), Cell Boost (HyClone), BalanCD CHO Feed (Irvine Scientific), BD Recharge (Becton Dickinson), Cellvento Feed (EMD Millipore), Ex-cell CHOZN Feed (Sigma-Aldrich), CHO Feed Bioreactor Supplement (Sigma-Aldrich), SheffCHO (Kerry), Zap-CHO (Invitria), ActiCHO (PAA/GE Healthcare), Ham's F10 (Sigma), Minimal Essential Medium ([MEM], Sigma), RPMI-1640 (Sigma), and Dulbecco's Modified Eagle's Medium ([DMEM], Sigma).

In some aspects, the serum-free growth second medium comprising no serum or serum at a concentration less than the first serum is substantially free (comprising no more than trace levels of) calcium ions, fetal bovine serum (FBS), fibronectin, collagen, laminin, or proteoglycans or non-proteoglycan polysaccharides of the extracellular matrix which would support the anchorage of cells. In one aspect, the second medium is a serum-free medium. In another aspect, the second medium comprises the serum at a concentration less than the serum concentration in the first medium.

In some aspects, the growth medium can have a pH of between about 6.5 and about 7.5, between about 6.5 and about 7.4, between about 6.5 and about 7.3, between about 6.5 and about 7.2, between about 6.5 and about 7.1, between about 6.5 and about 7.0, between about 6.5 and about 6.9, between about 6.5 and about 6.8, between about 6.5 and about 6.7, between about 6.6 and about 7.5, between about 6.6 and about 7.4, between about 6.6 and about 7.3, between about 6.6 and about 7.2, between about 6.6 and about 7.1, between about 6.6 and about 7.0, between about 6.6 and about 6.9, between about 6.6 and about 6.8, between about 6.7 and about 7.5, between about 6.7 and about 7.4, between about 6.7 and about 7.3, between about 6.7 and about 7.2, between about 6.7 and about 7.1, between about 6.7 and about 7.0, between about 6.7 and about 6.9, between about 6.8 and about 7.5, between about 6.8 and about 7.4, between about 6.8 and about 7.3, between about 6.8 and about 7.2, between about 6.8 and about 7.1, between about 6.8 and about 7.0, between about 6.9 and about 7.5, between about 6.9 and about 7.4, between about 6.9 and about 7.3, between about 6.9 and about 7.2, between about 6.9 and about 7.1, between about 7.0 and about 7.5, between about 7.0 and about 7.4, between about 7.0 and about 7.3, between about 7.0 and about 7.2, between about 7.1 and about 7.5, between about 7.1 and about 7.4, between about 7.1 and about 7.3, between about 7.2 and about 7.5, between about 7.2 and about 7.4, or between about 7.3 and about 7.5.

In some aspects, the cells can be cultured at a temperature of 32° C. to about 39° C., about 32° C. to about 37° C., between about 32° C. and about 37.5° C., between about 34° C. and about 37° C., between about 35° C. and about 37° C., between about 35.5° C. and about 37.5° C., between about 36° C. and about 37° C., or about 36.5° C. In some aspects, the cells can be incubated at a temperature of about 37° C. from the beginning to the end of the culturing period. In some aspects, the temperature can be changed or may vary slightly during the culturing period, e.g., on an hourly or daily basis. In some aspects, the temperature can be changed or shifted (e.g., increased or decreased) at about one day, two days, three days, four days, five days, six days, seven days, eight days, nine days, ten days, eleven days, twelve days, fourteen days, or fifteen days after the start of the culturing period, or at any time point within the culturing period. In some aspects, the temperature can be shifted upwards by about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0° C. In some aspects, the temperature can be shifted downwards by about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10° C.

In some aspects, the cell culture can be performed using an atmosphere containing about 1% to about 15% CO₂. In some aspects, cells can be cultured using an atmosphere containing about 14% CO₂, 12% CO₂, 10% CO₂, 8% CO₂, 6% CO₂, 5% CO₂, 4% CO₂, 3% CO₂, 2% CO₂, or about 1% CO₂.

In some aspects, the cell culture can be performed by maintaining a dissolved oxygen (dO₂) in the cell culture of between about 3% and about 55%, between about 3% and about 50%, between about 3% and about 45%, between about 3% and about 40%, between about 3% and about 35%, between about 3% and about 30%, between about 3% and about 25%, between about 3% and about 20%, between about 3% and about 15%, between about 5% and about 55%, between about 5% and about 50%, between about 5% and about 45%, between about 5% and about 40%, between about 5% and about 35%, between about 5% and about 30%, between about 5% and about 25%, between about 5% and about 20%, between about 5% and about 15%, between about 5% and about 10%, between about 10% and about 55%, between about 10% and about 50%, between about 10% and about 45%, between about 10% and about 40%, between about 10% and about 35%, between about 10% and about 30%, between about 10% and about 25%, between about 10% and about 20%, between about 15% and about 55%, between about 15% and about 50%, between about 15% and about 45%, between about 15% and about 40%, between about 15% and about 35%, between about 15% and about 30%, between about 15% and about 25%, between about 15% and about 20%, between about 20% and about 55%, between about 20% and about 50%, between about 20% and about 45%, between about 20% and about 40%, between about 20% and about 35%, between about 20% and about 30%, between about 20% and about 25%, between about 25% and about 55%, between about 25% and about 50%, between about 25% and about 45%, between about 25% and about 40%, between about 25% and about 35%, between about 25% and about 30%, between about 30% and about 55%, between about 30% and about 50%, between about 30% and about 45%, between about 30% and about 40%, between about 30% and about 35%, between about 35% and about 55%, between about 35% and about 50%, between about 35% and about 45%, between about 35% and about 40%, between about 40% and about 55%, between about 40% and about 50%, between about 40% and about 45%, between about 45% and about 55%, between about 45% and about 50%, or between about 50% and about 55%.

In some aspects, the pH of the cell culture can be maintained at a specific pH value by the addition of a base solution, such as an alkali base solution. The pH of a cell culture can be maintained at a pH of between about 6.5 and about 7.5, between about 6.5 and about 7.4, between about 6.5 and about 7.3, between about 6.5 and about 7.2, between about 6.5 and about 7.1, between about 6.5 and about 7.0, between about 6.5 and about 6.9, between about 6.5 and about 6.8, between about 6.5 and about 6.7, between about 6.6 and about 7.5, between about 6.6 and about 7.4, between about 6.6 and about 7.3, between about 6.6 and about 7.2, between about 6.6 and about 7.1, between about 6.6 and about 7.0, between about 6.6 and about 6.9, between about 6.6 and about 6.8, between about 6.7 and about 7.5, between about 6.7 and about 7.4, between about 6.7 and about 7.3, between about 6.7 and about 7.2, between about 6.7 and about 7.1, between about 6.7 and about 7.0, between about 6.7 and about 6.9, between about 6.8 and about 7.5, between about 6.8 and about 7.4, between about 6.8 and about 7.3, between about 6.8 and about 7.2, between about 6.8 and about 7.1, between about 6.8 and about 7.0, between about 6.9 and about 7.5, between about 6.9 and about 7.4, between about 6.9 and about 7.3, between about 6.9 and about 7.2, between about 6.9 and about 7.1, between about 7.0 and about 7.5, between about 7.0 and about 7.4, between about 7.0 and about 7.3, between about 7.0 and about 7.2, between about 7.1 and about 7.5, between about 7.1 and about 7.4, between about 7.1 and about 7.3, between about 7.2 and about 7.5, between about 7.2 and about 7.4, or between about 7.3 and about 7.5

In some aspects, cell culture under suspension conditions can be done in any type of cell culture flask suitable for steady or mixed/shaken suspension cell expansion using for example a T-flask, roller bottle, spinner flask or shaker flask; or combinations thereof. In some aspects, the N-1 container is a shake flask.

In some aspects, suspension conditions can include some form of agitation. In some aspects, the agitation can be rotary agitation. In some aspects, agitation can occur at a frequency of at about 25 RPM to about 500 RPM, between about 25 RPM and about 480 RPM, between about 25 RPM and about 460 RPM, between about 25 RPM and about 440 RPM, between about 25 RPM and about 420 RPM, between about 25 RPM and about 400 RPM, between about 25 RPM and about 380 RPM, between about 25 RPM and about 360 RPM, between about 25 RPM and about 340 RPM, between about 25 RPM and about 320 RPM, between about 25 RPM and about 300 RPM, between about 25 RPM and about 280 RPM, between about 25 RPM and about 260 RPM, between about 25 RPM and about 240 RPM, between about 25 RPM and about 220 RPM, between about 25 RPM and about 200 RPM, between about 25 RPM and about 180 RPM, between about 25 RPM and about 160 RPM, between about 25 RPM and about 140 RPM, between about 25 RPM and about 120 RPM, between about 25 RPM and about 100 RPM, between about 25 RPM and about 80 RPM, between about 25 RPM and about 60 RPM, between about 25 RPM and about 40 RPM, between about 25 RPM and about 35 RPM, between about 25 RPM and about 30 RPM, between about 50 RPM to about 500 RPM, between about 50 RPM and about 480 RPM, between about 50 RPM and about 460 RPM, between about 50 RPM and about 440 RPM, between about 50 RPM and about 420 RPM, between about 50 RPM and about 400 RPM, between about 50 RPM and about 380 RPM, between about 50 RPM and about 360 RPM, between about 50 RPM and about 340 RPM, between about 50 RPM and about 320 RPM, between about 50 RPM and about 300 RPM, between about 50 RPM and about 280 RPM, between about 50 RPM and about 260 RPM, between about 50 RPM and about 240 RPM, between about 50 RPM and about 220 RPM, between about 50 RPM and about 200 RPM, between about 50 RPM and about 180 RPM, between about 50 RPM and about 160 RPM, between about 50 RPM and about 140 RPM, between about 50 RPM and about 120 RPM, between about 50 RPM and about 100 RPM, between about 50 RPM and about 80 RPM, between about 50 RPM and about 60 RPM, between about 75 RPM to about 500 RPM, between about 75 RPM and about 480 RPM, between about 75 RPM and about 460 RPM, between about 75 RPM and about 440 RPM, between about 75 RPM and about 420 RPM, between about 75 RPM and about 400 RPM, between about 75 RPM and about 380 RPM, between about 75 RPM and about 360 RPM, between about 75 RPM and about 340 RPM, between about 75 RPM and about 320 RPM, between about 75 RPM and about 300 RPM, between about 75 RPM and about 280 RPM, between about 75 RPM and about 260 RPM, between about 75 RPM and about 240 RPM, between about 75 RPM and about 220 RPM, between about 75 RPM and about 200 RPM, between about 75 RPM and about 180 RPM, between about 75 RPM and about 160 RPM, between about 75 RPM and about 140 RPM, between about 75 RPM and about 120 RPM, between about 75 RPM and about 100 RPM, between about 75 RPM and about 80 RPM, between about 100 RPM to about 500 RPM, between about 100 RPM and about 480 RPM, between about 100 RPM and about 460 RPM, between about 100 RPM and about 440 RPM, between about 100 RPM and about 420 RPM, between about 100 RPM and about 400 RPM, between about 100 RPM and about 380 RPM, between about 100 RPM and about 360 RPM, between about 100 RPM and about 340 RPM, between about 100 RPM and about 320 RPM, between about 100 RPM and about 300 RPM, between about 100 RPM and about 280 RPM, between about 100 RPM and about 260 RPM, between about 100 RPM and about 240 RPM, between about 100 RPM and about 220 RPM, between about 100 RPM and about 200 RPM, between about 100 RPM and about 180 RPM, between about 100 RPM and about 160 RPM, between about 100 RPM and about 140 RPM, between about 100 RPM and about 120 RPM, between about 150 RPM to about 500 RPM, between about 150 RPM and about 480 RPM, between about 150 RPM and about 460 RPM, between about 150 RPM and about 440 RPM, between about 150 RPM and about 420 RPM, between about 150 RPM and about 400 RPM, between about 150 RPM and about 380 RPM, between about 150 RPM and about 360 RPM, between about 150 RPM and about 340 RPM, between about 150 RPM and about 320 RPM, between about 150 RPM and about 300 RPM, between about 150 RPM and about 280 RPM, between about 150 RPM and about 260 RPM, between about 150 RPM and about 240 RPM, between about 150 RPM and about 220 RPM, between about 150 RPM and about 200 RPM, between about 150 RPM and about 180 RPM, between about 150 RPM and about 160 RPM, between at about 200 RPM to about 500 RPM, between about 200 RPM and 480 RPM, between about 200 RPM and about 460 RPM, between about 200 RPM and about 440 RPM, between about 200 RPM and about 420 RPM, between about 200 RPM and about 400 RPM, between about 200 RPM and about 380 RPM, between about 200 RPM and about 360 RPM, between about 200 RPM and about 340 RPM, between about 200 RPM and about 320 RPM, between about 200 RPM and about 300 RPM, between about 200 RPM and about 280 RPM, between about 200 RPM and about 260 RPM, between about 200 RPM and about 240 RPM, between about 200 RPM and about 220 RPM, between about 240 RPM and about 500 RPM, between about 240 RPM and about 480 RPM, between about 240 RPM and about 460 RPM, between about 240 RPM and about 440 RPM, between about 240 RPM and about 420 RPM, between about 240 RPM and about 400 RPM, between about 240 RPM and about 380 RPM, between about 240 RPM and about 360 RPM, between about 240 RPM and about 340 RPM, between about 240 RPM and about 320 RPM, between about 240 RPM and about 300 RPM, between about 240 RPM and about 280 RPM, between about 240 RPM and about 260 RPM, between about 260 RPM and about 500 RPM, between about 260 RPM and about 480 RPM, between about 260 RPM and about 460 RPM, between about 260 RPM and about 440 RPM, between about 260 RPM and about 420 RPM, between about 260 RPM and about 400 RPM, between about 260 RPM and about 380 RPM, between about 260 RPM and about 360 RPM, between about 260 RPM and about 340 RPM, between about 260 RPM and about 320 RPM, between about 260 RPM and about 300 RPM, between about 260 RPM and about 280 RPM, between about 280 RPM and about 500 RPM, between about 280 RPM and about 480 RPM, between about 280 RPM and about 460 RPM, between about 280 RPM and about 440 RPM, between about 280 RPM and about 420 RPM, between about 280 RPM and about 400 RPM, between about 280 RPM and about 380 RPM, between about 280 RPM and about 360 RPM, between about 280 RPM and about 340 RPM, between about 280 RPM and about 320 RPM, between about 280 RPM and about 280 RPM, between about 300 RPM and about 500 RPM, between about 380 RPM and about 480 RPM, between about 380 RPM and about 460 RPM, between about 380 RPM and about 440 RPM, between about 380 RPM and about 420 RPM, between about 380 RPM and about 400 RPM, between about 400 RPM and about 500 RPM, between about 400 RPM and about 480 RPM, between about 400 RPM and about 460 RPM, between about 400 RPM and about 440 RPM, or between about 400 RPM and about 420 RPM. The agitation can be performed continuously or periodically.

In some aspects, the cells are passaged no more than two times under suspension conditions.

In some aspects, the cells are cultured in suspension culture for about 24 to about 96 hours. In some aspects, the cells are cultured in suspension culture for about 36 to about 84 hours. In some aspects, the cells are cultured in suspension culture for about 48 to about 72 hours. In some aspects, the cells are cultured in suspension culture for about 54 to about 66 hours. In some aspects, the cells are cultured in suspension culture for about 24, about 30, about 36, about 42, about 48, about 54, about 60, about 66, about 72, about 78, about 84, about 90, or about 96 hours.

In some aspects of the disclosure, the cells are adherent cells. In some aspects, the adherent cells are HeLa cells, CHO cells, HEK-293 cells, VERO cells, BHK cells, MDCK cells, MDBK cells, or COS cells. In some aspects, the adherent cell is human. In some aspects, the adherent cell is a HeLa or HEK-293 cell. In some aspects, the adherent cell is a HEK-293 cell.

In some aspects, the adherent cells are not suspension-adapted. In some aspects, culturing the cells under suspension conditions does not alter the adherent-dependency of the cells. In some aspects, the method does not alter the cells to create a new cell line. The methods disclosed herein do not alter the genomic or transcriptomic profile of the cells. The methods disclosed herein do not alter the phenotype of the cell.

In some aspects, the cells are passaged multiple times under adherent conditions in serum-supplemented growth medium prior to inoculating the N-1 container. In some aspects, the cells are cultured in a N-2, N-3, N-4, N-5, N-6, N-7, N-8, N-9, or N-10 containers prior to inoculating the N-1 container. In some aspects, the cells are cultured in a N-3 and N-2 container. In some aspects, the cells are cultured in a N-4, N-3, and N-2 container.

In some aspects, the bioreactor is an adherent bioreactor. In some aspects, the adherent cells are purified from the culture produced in the adherent bioreactor.

In some aspects, the bioreactor comprises at least one, more preferably a plurality of carriers onto which the expanded cells are intended to adhere, which may be either floating or fixed in the bioreactor. Preferably, said carriers can be made, for example, using polyethylene terephthalate, polystyrene, polyester, polypropylene, DEAE-dextran, collagen, glass, alginate or acrylamide. In some aspects, the bioreactor can be a bioreactor containing bead-type micro-carriers (e.g., Cytodex® brand beads, commercially available from GE Healthcare Inc. division of General Electric Corp.) or matrix type carriers (e.g., Fibra-Cell™ brand disks, commercially available from Eppendorf Corp.). In some aspects, the bioreactor uses a polyester fiber carrier such as that used in the iCELLis® nano or iCELLis® 500 bioreactors, (commercially available from Advanced Technology Materials Inc. (Brussels, Belgium) and Pall corporation (Fall River, Mass)).

In some aspects, the third medium in the bioreactor comprises at least one factor which promotes cell adherence. In some aspects, the at least one factor which promotes cell adherence is selected from the group consisting of FBS, fibronectin, collagen, laminin, calcium ions, proteoglycans or non-proteoglycan polysaccharides of the extracellular matrix; and combinations thereof. In some aspects, the at least one factor which promotes cell adherence can be added to the third medium just before, during, or after the inoculation of the suspension cells into bioreactor.

In some aspects the growth medium comprises DMEM and about 10% FBS by weight. In some aspects, the growth medium comprises about 2% to about 20% by weight FBS. In some aspects, the growth medium comprises about 3% to about 19% by weight of FBS. In some aspects, the growth medium comprises about 4% to about 18% by weight of FBS. In some aspects, the growth medium comprises about 5% to about 17% by weight of FBS. In some aspects, the growth medium comprises about 6% to about 16% by weight of FBS. In some aspects, the growth medium comprises about 7% to about 15% by weight of FBS. In some aspects, the growth medium comprises about 8% to about 14% by weight of FBS. In some aspects, the growth medium comprises about 9% to about 13% by weight of FBS. In some aspects, the growth medium comprises about 10% to about 12% by weight of FBS. In some aspects the growth medium comprises about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of FBS.

In some aspects, the suspension expanded cells from step (d) can be inoculated directly into the bioreactor. In some aspects, the quantity of cells inoculated into the bioreactor varies based on the size of the bioreactor. In some aspects, a 4 m2 bioreactor (e.g., iCELLis® nano bioreactor) is used. In some aspects, the 4 m2 bioreactor is inoculated with between about 1×10⁸ and 1×10⁹ cells. In some aspects, the 4 m2 bioreactor is inoculated with between about 3×10⁸ and 7×10⁸ cells. In some aspects, the 4 m2 bioreactor is inoculated with between about 4×10⁸ and 6×10⁸ cells. In some aspects, the 4 m2 bioreactor is inoculated with about 5×10⁸ cells. In some aspects, equivalent cell densities are used for other size bioreactors.

In some aspects, the method of the disclosure can further comprise culturing the cells in the bioreactor. In some aspects, the cell culture comprises batch culturing. In some aspects, the cell culture comprises fed-batch culturing. In some aspects, the cell culture comprises perfusion culturing.

Fed-batch culturing includes the incremental (periodic) or continuous addition of a feed culture medium to an initial cell culture without substantial or significant removal of the growth medium from the cell culture. The cell culture in fed batch culturing can be disposed in a bioreactor (e.g., a production bioreactor, such as a 10,000-L production bioreactor). In some aspects, the feed culture medium can be the same as the growth medium. The feed culture medium can be either in a liquid form or a dry powder. In some aspects, the feed culture medium is a concentrated form of the growth medium and/or is added as a dry powder. In some aspects, both a first liquid feed culture medium and a different second liquid feed culture medium can be added (e.g., continuously added) to the growth medium. In some aspects, the addition of the first liquid feed culture medium and addition of the second liquid feed culture medium to the culture can be initiated at about the same time. In some aspects, the total volume of the first liquid feed culture medium and the second liquid feed culture medium added to the culture over the entire culturing period can be about the same.

When the feed culture medium is added continuously, the rate of addition of the feed culture medium can be held constant or can be increased (e.g., steadily increased) over the culturing period. A continuous addition of feed culture medium can start at a specific time point during the culturing period (e.g., when the cells reach a target viable cell density, e.g., a viable cell density of about 1×106 cells/mL, about 1.1×106 cells/mL, about 1.2×106 cells/mL, about 1.3×106 cells/mL, about 1.4×106 cells/mL, about 1.5×106 cells/mL, about 1.6×106 cells/mL, about 1.7×106 cells/mL, about 1.8×106 cells/mL, about 1.9×106 cells/mL, or about 2.0×106 cells/mL). In some aspects, the continuous addition of feed culture medium can be initiated at day 2, day 3, day 4, or day 5 of the culturing period.

In some aspects, an incremental (periodic) addition of feed culture medium can begin when the cells reach a target cell density (e.g., about 1×106 cells/mL, about 1.1×106 cells/mL, about 1.2×106 cells/mL, about 1.3×106 cells/mL, about 1.4×106 cells/mL, about 1.5×106 cells/mL, about 1.6×106 cells/mL, about 1.7×106 cells/mL, about 1.8×106 cells/mL, about 1.9×106, or about 2.0×106 cells/mL). In some aspects, incremental feed culture media addition can occur at regular intervals (e.g., every day, every other day, or every third day) or can occur when the cells reach specific target cell densities (e.g., target cell densities that increase over the culturing period). In some aspects, the amount of feed culture medium added can progressively increase between the first incremental addition of feed culture medium and subsequent additions of feed culture medium. In some aspects, the volume of a liquid culture feed culture medium added to the initial cell culture over any 24 hour period in the culturing period can be some fraction of the initial volume of the bioreactor containing the culture or some fraction of the volume of the initial culture.

In some aspects, the addition of the liquid feed culture medium (continuously or periodically) can occur at a time point that is between 6 hours and 7 days, between about 6 hours and about 6 days, between about 6 hours and about 5 days, between about 6 hours and about 4 days, between about 6 hours and about 3 days, between about 6 hours and about 2 days, between about 6 hours and about 1 day, between about 12 hours and about 7 days, between about 12 hours and about 6 days, between about 12 hours and about 5 days, between about 12 hours and about 4 days, between about 12 hours and about 3 days, between about 12 hours and about 2 days, between about 1 day and about 7 days, between about 1 day and about 6 days, between about 1 day and about 5 days, between about 1 day and about 4 days, between about 1 day and about 3 days, between about 1 day and about 2 days, between about 2 days and about 7 days, between about 2 days and about 6 days, between about 2 days and about 5 days, between about 2 days and about 4 days, between about 2 days and about 3 days, between about 3 days and about 7 days, between about 3 days and about 6 days, between about 3 days and about 5 days, between about 3 days and about 4 days, between about 4 days and about 7 days, between about 4 days and about 6 days, between about 4 days and about 5 days, between about 5 days and about 7 days, or between about 5 days and about 6 days, after the start of the culturing period.

In some aspects, the volume of a liquid feed culture medium added (continuously or periodically) to the initial cell culture over any 24 hour period can be between 0.01× and about 0.3× of the capacity of the bioreactor. The fraction may be between about 0.01× and about 0.28×, between about 0.01× and about 0.26×, between about 0.01× and about 0.24×, between about 0.01× and about 0.22×, between about 0.01× and about 0.20×, between about 0.01× and about 0.18×, between about 0.01× and about 0.16×, between about 0.01× and about 0.14×, between about 0.01× and about 0.12×, between about 0.01× and about 0.10×, between about 0.01× and about 0.08×, between about 0.01× and about 0.06×, between about 0.01× and about 0.04×, between about 0.02× and about 0.3×, between about 0.02× and about 0.28×, between about 0.02× and about 0.26×, between about 0.02× and about 0.24×, between about 0.02× and about 0.22×, between about 0.02× and about 0.20×, between about 0.02× and about 0.18×, between about 0.02× and about 0.16×, between about 0.02× and about 0.14×, between about 0.02× and about 0.12×, between about 0.02× and about 0.10×, between about 0.02× and about 0.08×, between about 0.02× and about 0.06×, between about 0.02× and about 0.05×, between about 0.02× and about 0.04×, between about 0.02× and about 0.03×, between about 0.025× and about 0.3×, between about 0.025× and about 0.28×, between about 0.025× and about 0.26×, between about 0.025× and about 0.24×, between about 0.025× and about 0.22×, between about 0.025× and about 0.20×, between about 0.025× and about 0.18×, between about 0.025× and about 0.16×, between about 0.025× and about 0.14×, between about 0.025× and about 0.12×, between about 0.025× and about 0.10×, between about 0.025× and about 0.08×, between about 0.025× and about 0.06×, between about 0.025× and about 0.04×, between about 0.05× and about 0.3×, between about 0.05× and about 0.28×, between about 0.05× and about 0.26×, between about 0.05× and about 0.24×, between about 0.05× and about 0.22×, between about 0.05× and about 0.20×, between about 0.05× and about 0.18×, between about 0.05× and about 0.16×, between about 0.05× and about 0.14×, between about 0.05× and about 0.12×, between about 0.05× and about 0.10×, between about 0.1× and about 0.3×, between about 0.1× and about 0.28×, between about 0.1× and about 0.26×, between about 0.1× and about 0.24×, between about 0.1× and about 0.22×, between about 0.1× and about 0.20×, between about 0.1× and about 0.18×, between about 0.1× and about 0.16× between about 0.1× and about 0.14×, between about 0.1×, between about 0.15× and about 0.3×, between about 0.15× and about 0.2×, between about 0.2× and about 0.3×, or between about 0.25× and about 0.3×, of the capacity of the bioreactor.

In some aspects, the volume of a liquid feed culture medium added (continuously or periodically) to the initial cell culture over any 24 hour period during the culturing period can be between 0.02× and about 1.0×, between about 0.02× and about 0.9×, between about 0.02× and about 0.8×, between about 0.02× and about 0.7×, between about 0.02× and about 0.6×, between about 0.02× and about 0.5×, between about 0.02× and about 0.4×, between about 0.02× and about 0.3×, between about 0.02× and about 0.2×, between about 0.02× and about 0.1×, between about 0.02× and about 0.08×, between about 0.02× and about 0.06×, between about 0.02× and about 0.05×, between about 0.02× and about 0.04×, between about 0.02× and about 0.03×, between about 0.05× and about 1.0×, between about 0.05× and about 0.8×, between about 0.05× and about 0.7×, between about 0.05× and about 0.6×, between about 0.05× and about 0.5×, between about 0.05× and about 0.4×, between about 0.05× and about 0.3×, between about 0.05× and about 0.2×, between about 0.05× and about 0.1×, between about 0.1× and about 1.0×, between about 0.1× and about 0.9×, between about 0.1× and about 0.8×, between about 0.1× and about 0.7×, between about 0.1× and about 0.6×, between about 0.1× and about 0.5×, between about 0.1× and about 0.4×, between about 0.1× and about 0.3×, between about 0.1× and about 0.2×, between about 0.2× and about 1.0×, between about 0.2× and about 0.9×, between about 0.2× and about 0.8×, between about 0.2× and about 0.7×, between about 0.2× and about 0.6×, between about 0.2× and about 0.5×, or between about 0.2× and about 0.4× of the volume of the initial cell culture.

In some aspects, the total amount of feed culture medium added (continuously or periodically) over the entire culturing period can be between about 1% and about 40% (e.g., between about 1% and about 35%, between about 1% and about 30%, between about 1% and about 25%, between about 1% and about 20%, between about 1% and about 15%, between about 1% and about 10%, between about 1% and about 5%, between about 1% and about 4%, between about 2% and about 40%, between about 2% and about 35%, between about 2% and about 30%, between about 2% and about 25%, between about 2% and about 20%, between about 2% and about 15%, between about 2% and about 10%, between about 2% and about 5%, between about 3% and about 40%, between about 3% and about 35%, between about 3% and about 30%, between about 3% and about 25%, between about 3% and about 20%, between about 3% and about 15%, between about 3% and about 10%, between about 3% and about 5%, between about 4% and about 40%, between about 4% and about 35%, between about 4% and about 30%, between about 4% and about 25%, between about 4% and about 20%, between about 4% and about 15%, between about 4% and about 10%, between about 4% and about 8%, between about 5% and about 40%, between about 5% and about 35%, between about 5% and about 30%, between about 5% and about 25%, between about 5% and about 20%, between about 5% and about 15%, between about 5% and about 10%, between about 10% and about 40%, between about 10% and about 35%, between about 10% and about 30%, between about 10% and about 25%, between about 10% and about 20%, between about 10% and about 15%, between about 15% and about 40%, between about 15% and about 35%, between about 15% and about 30%, between about 15% and about 25%, between about 15% and about 20%, between about 20% and about 40%, between about 20% and about 35%, between about 20% and about 30%, between about 20% and about 25%, between about 25% and about 40%, between about 25% and about 35%, between about 25% and about 30%, between about 30% and about 40%, between about 30% and about 35%, or between about 35% and about 40%) of the volume of the initial culture.

In some aspects, two different feed culture media are added (continuously or incrementally) during feed batch culturing. In some aspects, the amount or volume of the first feed culture medium and the second feed culture medium added can be substantially the same or can differ. In some aspects, the first feed culture medium can be in the form of a liquid and the second feed culture medium can be in the form of a solid. In some aspects, the first feed culture medium and the second feed culture medium can be liquid feed culture media.

Perfusion culturing comprises removing from the bioreactor a first volume of the growth medium, and adding to the production bioreactor a second volume of a second growth culture medium, wherein the first volume and the second volume are about equal. The cells are retained in the bioreactor by a cell retention device or through techniques, such as cell settling in a settling cone. In some aspects, the removal and addition of growth media can be performed simultaneously or sequentially, or some combination of the two. In some aspects, removal and addition can be performed continuously, such as at a rate that removes and replaces a volume of between 0.1% to 800%, between 1% and 700%, between 1% and 600%, between 1% and 500%, between 1% and 400%, between 1% and 350%, between 1% and 300%, between 1% and 250%, between 1% and 100%, between 100% and 200%, between 5% and 150%, between 10% and 50%, between 15% and 40%, between 8% and 80%, or between 4% and 30% of the capacity of the bioreactor.

In some aspects, the first volume of the first growth medium removed and the second volume of the second growth medium added can be held approximately the same over each 24-hour period. In some aspects, the rate at which the first volume of the first growth medium is removed (volume/unit of time) and the rate at which the second volume of the second growth medium is added (volume/unit of time) can be varied and, depends on the conditions of the particular cell culture system. In some aspects, the rate at which the first volume of the first growth medium is removed (volume/unit of time) and the rate at which the second volume of the second growth medium is added (volume/unit of time) can be about the same or can be different.

In some aspects, the volume removed and added can change by gradually increasing over each 24-hour period. In some aspects, the volume of the first growth medium removed and the volume of the second growth medium added within each 24-hour period can be increased over the culturing period. In some aspects, the volume can be increased by a volume that is between 0.5% to about 20% of the capacity of the bioreactor over a 24-hour period. In some aspects, the volume can be increased over the culturing period to a volume that is about 25% to about 150% of the capacity of the bioreactor or the first liquid culture medium volume over a 24-hour period.

In some aspects, after the first 48 to 96 hours of the culturing period, in each 24-hour period, the first volume of the first growth medium removed and the second volume of the second growth medium added is about 10% to about 95%, about 10% to about 20%, about 20% to about 30%, about 30% to about 40%, about 40% to about 50%, about 50% to about 60%, about 60% to about 70%, about 70% to about 80%, about 80% to about 90%, about 85% to about 95%, about 60% to about 80%, or about 70% of the volume of the first growth medium.

In some aspects, the first growth medium and the second growth medium can be the same type of media. In some aspects, the first growth medium and the second growth medium can be different. In some aspects, the second liquid culture medium may be more concentrated with respect to one or more media components.

In some aspects, the first volume of the first growth medium can be removed by using any automated system. In some aspects, alternating tangential flow filtration may be used. In some aspects, the first volume of the first growth medium can be removed by seeping or gravity flow of the first volume of the first growth medium through a sterile membrane with a molecular weight cut-off that excludes the cell. In some aspects, the first volume of the first growth medium can be removed by stopping or significantly decreasing the rate of agitation for a period of at least 1 minute, at least 2 minutes, 3 minutes, 4 minutes, 5 minutes, 10 minutes, 15 minutes, 20 minutes, 25 minutes, 30 minutes, 40 minutes, 50 minutes, or 1 hour, and removing or aspirating the first volume of the growth medium from the top of the production bioreactor.

In some aspects, the second volume of the second liquid culture medium can be added to the first liquid culture medium by a pump. In some aspects, the second liquid culture medium can be added to the first liquid medium manually, such as by pipetting or injecting the second volume of the second liquid culture medium directly onto the first liquid culture medium or in an automated fashion.

In some aspects, the method further comprises contacting the cells with a first polynucleotide sequence. In some aspects, the method further comprises transfecting the cells with a polynucleotide sequence. In some aspects, the polynucleotide sequence is a plasmid. In some aspects, the plasmid encodes a capsid of a recombinant viral particle selected from the group consisting of an AAV, a lentivirus, a herpes virus, a polyoma virus, and a vaccinia virus. In some aspects, the cells are transfected before they are inoculated into the bioreactor. In some aspects, the cells are transfected after they are inoculated into the bioreactor. In some aspects, the cells are contacted or transfected with a second polynucleotide and comprises a nucleic acid encoding a transgene. In some aspects, the cells are cultured under conditions which produce the viral vector. In some aspects, the method further comprises isolating the produced viral vector.

In some aspects, the polynucleotide is a viral vector. In some aspects, the viral vector is an Adenoviral and Adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications (Russell, J. Gen. Virol. 81: 2573-2604 (2000)); Goncalves, Virol J. 2(1):43 2005)), as indicated above. AAV vectors can result in very stable long term expression of transgene expression (up to 9 years in dog (Niemeyer et al., Blood 113(4):797-806 (2009)) and up to 2 years in human (Nathwani et al., N Engl J Med. 365(25): 2357-2365 (2011); Simonelli et al., Mol Ther. 18(3):643-50 (2010), Epub 2009 Dec. 1)). In some aspects, adenoviral vectors are modified to reduce the host response as reviewed by Russell (2000, supra). Methods for gene therapy using AAV vectors are described by Wang et al., 2005, J Gene Med. March 9 (Epub ahead of print); Mandel et al., Curr Opin Mol Ther. 6:482-90 (2004); Martin et al., Eye 18:1049-55 (2004); Nathwani et al, N Engl J Med. 22; 365:2357-65 (2011); and Apparailly et al, Hum Gene Ther. 16(4):426-34 (2005).

In some aspects, the first polynucleotide sequence comprises one or more of an inverted terminal repeat, a nucleic acid encoding at least one AAV replication protein, a nucleic acid encoding at least one AAV packaging protein, a nucleic acid encoding at least one AAV structural capsid protein, or combinations thereof.

In some aspects, the cells are cultured under conditions which produce a recombinant viral particle. In some aspects, the method further comprises isolating the produced recombinant viral particle.

In some aspects, the viral vector comprises a transgene operably linked to appropriate regulatory sequences. The term “regulatory sequence” includes promoters, enhancers and other expression control elements (e.g., polyadenylation signals) that control the transcription or translation of the protein. Such regulatory sequences are described, for example, in Goeddel (Gene Expression Technology, Methods in Enzymology 185, Academic Press, San Diego, CA (1990)). In some aspects, the regulatory sequence can comprise a promoter sequence. In some aspects, the promoter sequence can be a cytomegalovirus (CMV) intermediate early promoter, viral long terminal repeat promoters (LTRs), such as those from murine moloney leukaemia virus (MMLV), rous sarcoma virus, or HTLV-1, the simian virus 40 (SV 40) early promoter, or the herpes simplex virus thymidine kinase promoter.

In some aspects, the viral vector includes a further nucleotide sequence coding for a further polypeptide. In some aspects, the further polypeptide can be a (selectable) marker polypeptide that allows for the identification, selection and/or screening for cells containing the viral vector. In some aspects, the marker polypeptide can be the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene. Sources for obtaining these marker genes and methods for their use are provided in Sambrook and Russel (2001) “Molecular Cloning: A Laboratory Manual (3^{rd} edition), Cold Spring Harbor Laboratory, Cold Spring Harbor Laboratory Press, New York.

### Method of Producing Viral Vectors (e.g., AAV)

Some aspects of the disclosure are directed to a method of producing a viral vector (e.g., rAAV disclosed herein) comprising expanding cells according to any one of the seed-train expansion methods disclosed herein, inoculating the growth medium in a bioreactor with the cells, transfecting the cells with a polynucleotide sequence encoding a viral particle, and culturing the cells in the bioreactor under conditions in which the viral particle is produced. In some aspects, production of viral vectors is disclosed in U.S. Application No. 63/123,602, which is expressly incorporated herein by reference.

The methods of introducing exogenous nucleic acid into host cells are well known in the art, and will vary with the host cell used. Techniques include, but are not limited to, dextran-mediated transfection, calcium phosphate precipitation, calcium chloride treatment, polyethylenimine mediated transfection, polybrene mediated transfection, protoplast fusion, electroporation, viral or phage infection, encapsulation of the polynucleotide(s) in liposomes, and direct microinjection of the DNA into nuclei. Transfection can be either transient or stable.

In some aspects, the polynucleotide sequence is a plasmid. In some aspects, the plasmid encodes a viral particle from AAV.

In some aspects, the polynucleotide sequence is a viral vector. In some aspects, the viral vector encodes a viral particle. In preferred aspects, the viral particle is from AAV. In some aspects, the rAAV comprises the nucleic acid sequence of SEQ ID NO: 9. In some aspects, the present disclosure provides rAAV particles comprising the nucleic acid sequence of SEQ ID NO: 9. In some aspects, the present disclosure provides rAAV comprising nucleotides 55-5021 of SEQ ID NO: 3. In some aspects, the present disclosure provides rAAV particles comprising nucleotides 55-5021 of SEQ ID NO: 3. In some aspects, the rAAV comprises nucleotides 1-4988 of SEQ ID NO: 8. In some aspects, the present disclosure provides rAAV particles comprising nucleotides 1-4977 of SEQ ID NO: 8.

In some aspects, the viral vector is an AAV vector. In some aspects, the AAV vector can comprise a recombinant AAV vector (rAAV). An “rAAV vector” as used herein refers to a recombinant vector comprising part of an AAV genome encapsidated in a protein shell of capsid protein derived from an AAV serotype as disclosed herein. In some aspects, the AAV vector can comprise inverted terminal repeats (ITR) derived from an adeno-associated virus serotype, such as AAV1, AAV2, AAV3, AAV4, AAV5, AAV6, AAV7, AAV8, AAVrh8, AAV9, AAV10, AAVRH10, AAV11, AAV12, and others.

Typically, a vector genome requires the use of flanking 5′ and a 3′ ITR sequences to allow for efficient packaging of the vector genome into the rAAV capsid. In some aspects, the rAAV genome present in a rAAV vector comprises at least the nucleotide sequences of the inverted terminal repeat regions (ITR) of one of the AAV serotypes, or nucleotide sequences substantially identical thereto, and nucleic acid sequence encoding a transgene under control of a suitable regulatory element (e.g., a promoter), wherein the regulatory element and modified nucleic acid sequence(s) are inserted between the two ITRs.

The complete genome of several AAV serotypes and corresponding ITR has been sequenced (Chiorini et al. J. of Virology 73: 1309-1319 (1999)). They can be either cloned or made by chemical synthesis as known in the art, using for example an oligonucleotide synthesizer as supplied, e.g., by Applied Biosystems Inc. (Fosters, Calif., USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the wild type AAV sequence between the ITRs can be replaced with the desired nucleotide sequence.

In some aspects, the viral capsid component of the packaged viral vectors can be a parvovirus capsid, e.g., AAV Cap and/or chimeric capsids. Examples of suitable parvovirus viral capsid components are capsid components from the family Parvoviridae, such as an autonomous parvovirus or a Dependovirus. For example, the viral capsid may be an AAV capsid (e.g., AAV1, AAV2, AAV3, AAV4, AAV5, AAV6, AAV7, AAV8, AAVRH8 AAV9, AAV10, AAVRH10, AAV11 or AAV12 capsid; one skilled in the art would know there are likely other variants not yet identified that perform the same or similar function), or may include components from two or more AAV capsids. A full complement of AAV Cap proteins includes VP1, VP2, and VP3. The ORF comprising nucleotide sequences encoding AAV VP capsid proteins can comprise less than a full complement AAV Cap proteins or the full complement of AAV Cap proteins can be provided.

In some aspects, one or more of the AAV Cap proteins can be a chimeric protein, including amino acid sequences AAV Caps from two or more viruses, preferably two or more AAVs. For example, the chimeric virus capsid can include an AAV1 Cap protein or subunit and at least one AAV2 Cap or subunit. In some aspects, the rAAV genome as present in a rAAV vector does not comprise any nucleotide sequences encoding viral proteins, such as the rep (replication) or cap (capsid) genes of AAV. In some aspects, the rAAV genome can further comprise a marker or reporter gene, such as a gene for example encoding an antibiotic resistance gene, a fluorescent protein (e.g., gfp) or a gene encoding a chemically, enzymatically or otherwise detectable and/or selectable product (e.g., lacZ, aph, etc.) known in the art.

In some aspects, the rAAV genome as present in said rAAV vector can further comprise a promoter sequence operably linked to the nucleotide sequence encoding a transgene.

In some aspects, suitable 3′ untranslated sequence can also be operably linked to the modified nucleic acid sequences encoding a transgene. Suitable 3′ untranslated regions can be those naturally associated with the nucleotide sequence or can be derived from different genes, such as for example the bovine growth hormone 3′ untranslated region (e.g., bGH polyadenylation signal, SV40 polyadenylation signal, SV40 polyadenylation signal and enhancer sequence).

Except as otherwise indicated, methods known to those skilled in the art may be used for the construction of recombinant parvovirus and AAV (rAAV) constructs, packaging vectors expressing the parvovirus Rep and/or Cap sequences, and transiently and stably transacted packaging cells. Such techniques are known to those skilled in the art. See, e.g., SAMBROOK et al., MOLECULAR CLONING: A LABORATORY MANUAL 2nd Ed. (Cold Spring Harbor, N.Y., 1989); AUSUBEL el al., CURRENT PROTOCOLS IN MOLECULAR BIOLOGY (Green Publishing Associates, Inc. and John Wiley Sons, Inc., New York).

In some aspects, suitable 3′ untranslated sequence can also be operably linked to the nucleic acid sequences encoding a transgene. Suitable 3′ untranslated regions can be those naturally associated with the nucleotide sequence or can be derived from different genes, such as for example the bovine growth hormone 3′ untranslated region (e.g., bGH polyadenylation signal, SV40 polyadenylation signal, SV40 polyadenylation signal and enhancer sequence).

In some aspects, additional nucleotide sequences can be operably linked to the nucleic acid sequence encoding a transgene, such as nucleotide sequences encoding signal sequences, nuclear localization signals, expression enhancers, and the like.

Except as otherwise indicated, methods known to those skilled in the art may be used for the construction of lentiviral constructs, vectors, and transiently and stably transacted packaging cells. Such techniques are known to those skilled in the art. See, e.g., SAMBROOK et al., MOLECULAR CLONING: A LABORATORY MANUAL 2nd Ed. (Cold Spring Harbor, N.Y., 1989); AUSUBEL el al., CURRENT PROTOCOLS IN MOLECULAR BIOLOGY (Green Publishing Associates, Inc. and John Wiley Sons, Inc., New York).

In some aspects, the methods according to the present disclosure comprise transfecting cells with a transgene plasmid comprising a rAAVrh74.MHCK7.microdystrophin construct, a plasmid comprising an AAV rep gene and an AAV cap gene, and an adenovirus helper plasmid. In some aspects, the transgene plasmid comprising a rAAVrh74.MHCK7.microdystrophin construct comprises: the nucleic acid sequence of SEQ ID NO: 9, nucleotides 55-5021 of SEQ ID NO: 3, or nucleotides 1-4977 of SEQ ID NO: 8. In some aspects, the plasmid comprising an AAV rep gene and an AAV cap gene comprises an AAV2 rep gene and an rAAVrh74 cap gene. In some aspects, the adenovirus helper plasmid comprises an adenovirus 5 E2A, E4ORF6, and a VA RNA gene.

In some aspects, the method further comprises isolating the produced viral particle. The viral vector replicates inside the cell and is thereby amplified and produces viral particles. Viral infection results in the lysis of the transfected cells. The lytic characteristics of viral vectors such as, AAV, therefore permits two different modes of virus particle production and isolation. The first mode is harvesting virus particles prior to cell lysis, employing external factors to lyse the cells. The second mode is harvesting virus particles from supernatant after almost complete cell lysis by the produced virus.

Methods that can be used for active cell lysis are known to the person skilled in the art. In some aspects, cells can be lysed by freeze-thaw, solid shear, hypertonic and/or hypotonic lysis, liquid shear, sonication, high-pressure extrusion, detergent lysis, combinations of the above, and the like.

In some aspects, the cells can be lysed using at least one detergent. In some aspects, the detergent can include anionic, cationic, zwitterionic, and nonionic detergents. In some aspects, the concentration of the detergent can be about 0.1%-5% (w/w). In some aspects, the detergent can be Triton X-100.

In some aspects, nuclease can be employed to remove contaminating nucleic acids, i.e., native nucleic acids from transfected cells. In some aspects, the nuclease can be BENZONASE®, PULMOZYME®, or any other DNase and/or RNase commonly used in the art.

Methods for harvesting or isolating viral vectors from transfected cells have been extensively disclosed in WO 2005/080556, which is incorporated herein by reference in its entirety.

In some aspects, the time of harvest or isolation of the viral vector is between about 24 and 120 hours post transfection, between about 36 and 108 hours, between about 48 and about 96 hours post transfection, between about 60 and about 84 hours post transfection. In some aspects, the time of harvest or isolation of the vector is about 72 hours post transfection.

In some aspects, the isolated viral particle can be further purified. In some aspects, purification of the viral particles can be performed in several steps comprising clarification, ultrafiltration, diafiltration or separation with chromatography. Such methods have been described in WO 2005/080556, incorporated herein by reference in its entirety. In some aspects, clarification may be done by a filtration step, removing cell debris and other impurities from the cell lysate. In some aspects, ultrafiltration is used to concentrate the virus solution. In some aspects, diafiltration, buffer exchange, or ultrafilters can be used to remove and exchange salts, sugars and the like. The person skilled in the art knows how to find the optimal conditions for each purification step.

In some aspects, purification can be achieved by density gradient centrifugation. In some aspects, purification employs at least one chromatography step. In some aspects, the viral vector can be purified by anion exchange chromatography, size exclusion chromatography, or a combination thereof.

### Methods of Treating Muscular Dystrophy (e.g., DMD)

The present disclosure provides for methods of treating muscular dystrophy in a human subject in need thereof comprising the step of administering a recombinant adeno-associated virus (rAAV) rAAV.MHCK7.microdystrophin, wherein the rAAV is administered by a systemic route of administration at a dose of about 5.0×10¹² vg/kg to about 1.0×10¹⁵ vg/kg, and wherein the rAAV is produced in mammalian adherent cells, and wherein the adherent cells are cultured in an N-1 container under suspension conditions. In some aspects, the muscular dystrophy is Duchenne muscular dystrophy or Becker's muscular dystrophy. In some aspects, the muscular dystrophy is Duchenne muscular dystrophy.

The present disclosure also provides a method of treating muscular dystrophy in a human subject in need thereof comprising administering the composition comprising the rAAV described herein to said human subject. In some aspects, the rAAV is administered using a systemic route of administration and at a dose of about 5.0×10¹² vg/kg to about 1.0×10¹⁵ vg/kg. In some aspects, the systemic route of administration is an intravenous route and the dose of the rAAV administered is about 2×10¹⁴ vg/kg.

In some aspects, the dose of rAAV is administered at a concentration of about 10 mL/kg. In some aspects, the rAAV is administered by injection, infusion, or implantation. In some aspects, the rAAV is administered by infusion over approximately one hour. In some aspects, the rAAV is administered by an intravenous route through a peripheral limb vein.

In some aspects, the muscular dystrophy is Duchenne muscular dystrophy or Becker's muscular dystrophy. In some aspects, the muscular dystrophy is Duchenne muscular dystrophy.

In some aspects, the level of micro-dystrophin gene expression in a cell of the subject is increased after administration of the rAAV as compared to the level of micro-dystrophin gene expression before administration of the rAAV. In some aspects, expression of the micro-dystrophin gene in the cell is detected by measuring the micro-dystrophin protein level by Western blot in muscle biopsied before and after administration of the rAAV. In some aspects, expression is at least 55.4% after administration of the rAAV, as compared to before.

In some aspects, the mean percentage of micro-dystrophin positive fibers in the muscle tissue of the subject is increased after administration of the rAAV, as compared to the number of micro-dystrophin positive fibers before administration of the rAAV. In some aspects, the mean percentage of micro-dystrophin positive fibers is at least 70.5% and the mean intensity is at least 116.9% as detected by immunofluorescence (IF) in muscle biopsies before and after administration of the rAAV. In some aspects, micro-dystrophin transduction by vector genome count is at least 3.87 mean vector genome copies per nucleus.

In some aspects, in a method of treating a patient with DMD, the composition is administered to a genotyped patient. In some aspects, the patient's human dystrophin (DMD) gene is genotyped. In some aspects, the genotyped patient is genotyped for at least one mutation in exons 18-79 of the human dystrophin (DMD) gene.

In some aspects, the method of treating muscular dystrophy further comprises genotyping the DMD gene of the human subject prior to administering the composition to said human subject. In some aspects, the genotyping detects at least one mutation in exons 18 to 79 of the DMD gene. In some aspects, the at least one mutation is a frameshift deletion, a frameshift duplication, a premature stop, or other pathogenic variant resulting in the absence of expression of the human dystrophin protein.

In some aspects, detecting a frameshift deletion, a frameshift duplication, a premature stop, or other pathogenic variant resulting in the absence of expression of the human dystrophin protein identifies that a subject is eligible to be administered the compositions disclosed herein.

In one aspect, the at least one mutation in the DMD gene is a mutation in exons 1-17, an in-frame deletion, an in-frame duplication, a variant of uncertain significance (VUS), or a mutation fully contained in exon 45, thereby identifying that a subject is not eligible to be administered the compositions disclosed herein.

The present disclosure also provides a use of the composition described herein for the treatment of muscular dystrophy in a human subject in need thereof. In some aspects, the present disclosure. The present disclosure also provides a use of the composition described herein in the manufacture of a medicament for the treatment of muscular dystrophy.

In some aspects, the muscular dystrophy is Duchenne muscular dystrophy or Becker's muscular dystrophy. In some aspects, the muscular dystrophy is Duchenne muscular dystrophy.

For example, the dose of rAAV administered is about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg.

In some aspects, the human subject is between about 4 years old and less than 8 years old. In some aspects, the human subject is about 4, about 4.25, about 4.5, about 4.75, about 5, about 5.25, about 5.5, about 5.75, about 6, about 6.25, about 6.5 about 6.75, about 7, about 7.25, about 7.5, or about 7.75 years old.

In some aspects, the human subject is between about 8 years old and less than 18 years old. In some aspects, the human subject is about 8, about 8.25, about 8.5, about 8.75, about 9, about 9.25, about 9.5, about 9.75, about 10, about 10.25, about 10.5, about 10.75, about 11, about 11.25, about 11.5, about 11.75, about 12, about 12.25, about 12.5, about 12.75, about 13, about 13.25, about 13.5, about 13.75, about 14, about 14.5, about 14.75, about 15, about 15.25, about 15.5, about 15.75, about 16, about 16.25, about 16.5, about 16.75, about 17, about 17.25, about 17.5, or about 17.75 years old.

In one aspect, the methods of the present disclosure comprise systemically administering rAAV wherein the systemic route of administration is an intravenous route and the dose of the rAAV administered is about 2.0×10¹⁴ vg/kg. In another aspect, the methods of the present disclosure comprise systemically administering rAAV wherein the systemic route of administration is an intravenous route and the dose of the rAAV administered is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵ vg/kg. In one aspect, the rAAV is AAVrh74.MHCK7.microdystrophin or AAVrh74.MCK.microdystrophin. In one aspect, the rAAV is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one aspect, the rAAV is the AAVrh74.MCK.microdystrophin of nucleotides 56-4820 of SEQ ID NO: 5.

In any of the methods of the present disclosure, the dose of rAAV can be administered at about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/k, or about 10 mL/kg to 15 mL/kg or 10 mL/kg to about 20 mL/kg. In a particular aspect, the dose or the rAAV is administered in about 10 mL/kg. In one aspect, the rAAV is AAVrh74.MHCK7.microdystrophin or AAVrh74.MCK.microdystrophin. In one aspect, the rAAV is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one aspect, the rAAV is the AAVrh74.MCK.microdystrophin of nucleotides 56-4820 of SEQ ID NO: 5.

In any of the methods of the present disclosure, the dose of rAAV can be administered by injection, infusion or implantation. For example, the dose of rAAV is administered by infusion over approximately one hour. In addition, the dose of rAAV is administered by an intravenous route through a peripheral limb vein, such as a peripheral arm vein or a peripheral leg vein. Alternatively, the infusion may be administered over approximately 30 minutes, or approximately 1.5 hours, or approximately 2 hours, or approximately 2.5 hours or approximately 3 hours. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

The rAAV administered by any of the methods of the present disclosure can comprise the human micro-dystrophin nucleotide sequence of SEQ ID NO: 1, the MHCK7 promoter sequence of SEQ ID NO: 2 or SEQ ID NO:7. In addition, the rAAV administered by any of the methods of the present disclosure comprises the human micro-dystrophin nucleotide sequence of SEQ ID NO: 1 and the MHCK7 promoter sequence of SEQ ID NO: 2 or SEQ ID NO:7. For example, the rAAV can comprise the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6.

In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In any of the methods of the present disclosure, the rAAV administered is of the serotype AAVrh7.4.

In some aspects, the methods of the present disclosure treat Duchenne muscular dystrophy or Becker's muscular dystrophy. An exemplary aspect is a method of treating Duchenne muscular dystrophy or Becker's muscular dystrophy in a human subject in need thereof comprising the step of administering a dose of recombinant adeno-associated virus (rAAV) rAAV.MHCK7.microdystrophin, wherein the route of administration is intravenous infusion and the dose of the rAAV administered is about 2×10¹⁴ vg/kg over approximately one hour, and wherein the rAAV vector comprises the AAVrh74.MHCK7.microdystrophin construct nucleotide sequence of SEQ ID NO: 9 or of nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9 or of nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In any of the methods of treating a muscular dystrophy, the level of micro-dystrophin gene expression in a cell of the subject is increased after administration of the rAAV. Expression of the micro-dystrophin gene in the cell is detected by measuring the micro-dystrophin protein level by Western blot in muscle biopsied before and after administration of the rAAV. In particular, the level of micro-dystrophin protein is increased by at least about 70% to at least about 80%, or at least about 70% to at least about 90%, or at least about 80% to at least about 90% after administration of rAAV compared to the level of micro-dystrophin before administration of rAAV. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of rAAV compared to the level of micro-dystrophin before administration of rAAV.

In addition, expression of the micro-dystrophin gene in the cell is detected by measuring the micro-dystrophin protein level by immunohistochemistry in muscle biopsies before and after administration of the rAAV. The level of micro-dystrophin protein is increased by at least about 70% to at least about 80%, or at least about 70% to at least about 90%, or at least about 80% to at least about 90% after administration of rAAV compared to the level of micro-dystrophin before administration of rAAV. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of rAAV compared to the level of micro-dystrophin before administration of rAAV.

In any of the methods of treating a muscular dystrophy, the serum CK level in the subject is decreased after administration of the rAAV as compared to serum CK level before administration of the rAAV. For example, the serum CK level in the subject is decreased by about 65% to about 90% or about 65% to about 95% or about 75% to about 90% or about 80% to about 90% or about 85% to about 95% or about 87% to about 95% or about 87% to about 90% by 60 days after administration of the rAAV as compared to the serum CK level before administration of the rAAV. In particular, in any of the methods of treating a muscular dystrophy of the present disclosure, the serum CK level in the subject is decreased by about 87% by 60 days after administration of the rAAV as compared to the serum CK level before administration of the rAAV or in any of the methods of treating a muscular dystrophy of the present disclosure, the serum CK level in the subject is decreased by about 72% by 60 days after administration of the rAAV as compared to the serum CK level before administration of the rAAV, or in any of the methods of treating a muscular dystrophy of the present disclosure, the serum CK level in the subject is decreased by about 73% by 60 days after administration of the rAAV as compared to the serum CK level before administration of the rAAV, or in any of the methods of treating a muscular dystrophy of the present disclosure, the serum CK level in the subject is decreased by about 78% by 60 days after administration of the rAAV as compared to the serum CK level before administration of the rAAV. or in any of the methods of treating a muscular dystrophy of the present disclosure, the serum CK level in the subject is decreased by about 95% by 60 days after administration of the rAAV as compared to the serum CK level before administration of the rAAV. In any of the methods of treating a muscular dystrophy, the number of micro-dystrophin positive fibers in the muscle tissue of the subject is increased after administration of the rAAV as compared to the number of micro-dystrophin positive fibers before administration of the rAAV. For example, the number of micro-dystrophin positive fibers is detected by measuring the micro-dystrophin protein level by Western blot or immunohistochemistry on muscle biopsies before and after administration of the rAAV.

In any of the methods of treating muscular dystrophy, as described herein, administration of the rAAV upregulates expression of DAPC proteins such as alpha-sarcoglycan or beta-sarcoglycan. For example, the level of alpha-sarcoglycan in the subject is increased after administration of the rAAV as compared to the level of alpha-sarcoglycan before administration of the rAAV. In addition, the level of beta-sarcoglycan in the subject is increased after administration of the rAAV as compared to the level of the beta-sarcoglycan before administration of the rAAV. The level of alpha-sarcoglycan or beta-sarcoglycan is detected by measuring the alpha-sarcoglycan or beta-sarcoglycan protein level by Western blot or immunohistochemistry on muscle biopsies before and after administration of the rAAV.

In any of the methods of treating muscular dystrophy, disease progression in the subject is delayed after administration of the rAAV as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, in any of the methods, the subject has at least a 1.5, 2.0, 2.5, 2.6, 2.7, 2.8, 2.9, 3.0, 3.1, 3.2, 3.3, 3.4, 3.5, 4.0, 4.5, 5.0, 5.5, 6.0, 6.1, 6.2, 6.3, 6.4, or a 6.5 point improvement in NSAA score at least 270 days after administration of the rAAV as compared to NSAA score before administration of the rAAV. Further, in any of the methods, the subject has at least about 0.8 second improvement in time to rise at least 270 days after administration of the rAAV as compared to time to rise before administration of the rAAV. In addition, in any of the methods, the subject has at least about 1.2 second improvement in time to ascend 4 steps test at least 270 days after administration of the rAAV as compared to time to ascend 4 steps test before administration of the rAAV. In addition, in any of the methods, the subject has at least about 7 second improvement in the 100 m timed test at least 270 days after administration of the rAAV as compared to the 100 m timed test before administration of the rAAV.

In another aspect, the present disclosure provides for methods of expressing micro-dystrophin gene in a patient cell comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6. For example, expression of the micro-dystrophin gene in the patient cell is detected by measuring the micro-dystrophin protein level by Western blot or immunohistochemistry in muscle biopsies before and after administration of the rAAV.MHCK7.micro-dystrophin construct. In addition, the expression of the micro-dystrophin gene is measured in the patient by detecting greater the number of vector genomes per nucleus, wherein 1 vector genome per nucleus is about 50% micro-dystrophin expression and greater than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In some aspects, the present disclosure provides for methods of decreasing serum CK levels in a patient in need thereof, the method comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the serum CK level in the patient is decreased by at least about 65% to about 90% or about 65% to about 95% or about 75% to about 90% or about 80% to about 90% or about 85% to about 95% or about 87% to about 95% or about 87% to about 90% by 60 days after administration of the rAAV as compared to the serum CK level before administration of the rAAV. In particular, the serum CK level in the subject is decreased by about 87% by 60 days after administration of the rAAV as compared to the serum CK level before administration of the rAAV or in any of the methods of treating a muscular dystrophy of the present disclosure, the serum CK level in the subject is decreased by about 72% by 60 days after administration of the rAAV as compared to the serum CK level before administration of the rAAV, or in any of the methods of treating a muscular dystrophy of the present disclosure, the serum CK level in the subject is decreased by about 73% by 60 days after administration of the rAAV as compared to the serum CK level before administration of the rAAV, or in any of the methods of treating a muscular dystrophy of the present disclosure, the serum CK level in the subject is decreased by about 78% by 60 days after administration of the rAAV as compared to the serum CK level before administration of the rAAV, or in any of the methods of treating a muscular dystrophy of the present disclosure, the serum CK level in the subject is decreased by about 95% by 60 days after administration of the rAAV as compared to the serum CK level before administration of the rAAV.

The present disclosure also provides for methods of increasing micro-dystrophin positive fibers in a patient muscle tissue comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the number of micro-dystrophin positive fibers is detected by measuring the dystrophin protein level by Western blot or immunohistochemistry on muscle biopsies before and after administration of the rAAV. In addition, the expression of the micro-dystrophin gene is measured in the patient by detecting greater the number of vector genomes per nucleus, wherein 1 vector genome per nucleus is about 50% micro-dystrophin expression and greater than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In another aspect, the present disclosure provides for methods of increasing the expression of alpha-sarcoglycan in a patient in need thereof comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the level of alpha-sarcoglycan is detected by measuring the alpha-sarcoglycan protein level by Western blot or immunohistochemistry on muscle biopsies before and after administration of the rAAV.

In addition, the present disclosure provides for methods of increasing the expression of beta-sarcoglycan in a patient in need thereof comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the level of beta-sarcoglycan is detected by measuring the beta-sarcoglycan protein level by Western blot or immunohistochemistry on muscle biopsies before and after administration of the rAAV.

The present disclosure also provides for methods of treating a patient with Duchenne muscular dystrophy or Becker muscular dystrophy comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6, such that disease progression in the patient is delayed as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, in any of the methods, the subject has at least a 1.5, 2.0, 2.5, 2.6, 2.7, 2.8, 2.9, 3.0, 3.1, 3.2, 3.3, 3.4, 3.5, 4.0, 4.5, 5.0, 5.5, 6.0, 6.1, 6.2, 6.3, 6.4, or a 6.5 point improvement in NSAA score at least 270 days after administration of the rAAV as compared to NSAA score before administration of the rAAV. Further, in any of the methods, the subject has at least about 0.8 second improvement in time to rise at least 270 days after administration of the rAAV as compared to time to rise before administration of the rAAV. In addition, in any of the methods, the subject has at least about 1.2 second improvement in time to ascend 4 steps test at least 270 days after administration of the rAAV as compared to time to ascend 4 steps test before administration of the rAAV. In addition, in any of the methods, the subject has at least about 7 second improvement in the 100 m timed test at least 270 days after administration of the rAAV as compared to the 100 m timed test before administration of the rAAV.

“Fibrosis” refers to the excessive or unregulated deposition of extracellular matrix (ECM) components and abnormal repair processes in tissues upon injury, including skeletal muscle, cardiac muscle, liver, lung, kidney, and pancreas. The ECM components that are deposited include fibronectin and collagen, e.g. collagen 1, collagen 2 or collagen 3.

The present disclosure also provides for methods of reducing or preventing fibrosis in a subject suffering from muscular dystrophy comprising administering a therapeutically effective amount of a rAAV comprising the human micro-dystrophin nucleotide sequence of SEQ ID NO: 1 and the MHCK7 promoter nucleotide sequence of SEQ ID NO: 2 or SEQ ID NO: 7; or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MHCK7.microdystrophin. In one aspect, the AAVrh74.MHCK7.microdystrophin is the AAVrh74.MHCK7.microdystrophin of nucleotides 55-5021 of SEQ ID NO: 3. In another aspect, the AAVrh74.MHCK7.microdystrophin is the AAVrh74.MHCK7.microdystrophin of SEQ ID NO: 9. In another aspect, the AAVrh74.MHCK7.microdystrophin is the AAVrh74.MHCK7.microdystrophin of nucleotides 1-4977 of SEQ ID NO: 8 or nucleotides 56-5066 of SEQ ID NO: 6. In a further aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In another aspect, the present disclosure provides for methods of preventing fibrosis in a subject in need thereof, comprising administering a therapeutically effective amount of the human micro-dystrophin nucleotide sequence of SEQ ID NO: 1 and the MHCK7 promoter nucleotide sequence of SEQ ID NO: 2 or SEQ ID NO:7; or rAAV vector comprising the AAV74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, any of the rAAV of the present disclosure can be administered to subjects suffering from muscular dystrophy to prevent fibrosis, e.g. the rAAV of the present disclosure expressing a human micro-dystrophin protein administered before fibrosis is observed in the subject. In addition, the rAAV of the present disclosure expressing a human micro-dystrophin gene can be administered to a subject at risk of developing fibrosis, such as those suffering or diagnosed with muscular dystrophy, e.g. DMD. The rAAV of the present disclosure can be administered to the subject suffering from muscular dystrophy in order to prevent new fibrosis in these subjects.

The present disclosure contemplates administering rAAV before fibrosis is observed in the subject. In addition, the rAAV can be administered to a subject at risk of developing fibrosis, such as those suffering or diagnosed with a muscular dystrophy, e.g. DMD. The rAAV can be administered to the subject suffering from muscular dystrophy who already has developed fibrosis in order to prevent new fibrosis in these subjects.

The present disclosure also provides for methods of increasing muscular force and/or muscle mass in a subject suffering from a muscular dystrophy comprising administering a therapeutically effective amount of the human micro-dystrophin nucleotide sequence of SEQ ID NO: 1 and the MHCK7 promoter nucleotide sequence of SEQ ID NO: 2 or SEQ ID NO: 7; or a rAAV comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

The present disclosure contemplates administering rAAV vectors to subjects diagnosed with DMD before fibrosis is observed in the subject or before the muscle force has been reduced or before the muscle mass has been reduced.

The present disclosure also contemplates administering the human micro-dystrophin nucleotide sequence of SEQ ID NO: 1 and the MHCK7 promoter nucleotide sequence of SEQ ID NO: 2 or SEQ ID NO:7; or a rAAV comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 to a subject suffering from a muscular dystrophy who already has developed fibrosis, in order to prevent new fibrosis in these subjects or to reduce fibrosis in these subjects. The present disclosure also provides for administering the human micro-dystrophin nucleotide sequence of SEQ ID NO: 1 and the MHCK7 promoter nucleotide sequence of SEQ ID NO: 2 or SEQ ID NO:7; or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 to the subject suffering from a muscular dystrophy who already has reduced muscle force or has reduced muscle mass in order to protect the muscle from further injury.

The present disclosure also provides for methods of treating cardiomyopathy in a human subject with muscular dystrophy (e.g., DMD), comprising administering any of the compositions described herein (e.g., delandistrogene moxeparvovec) to said human subject. In some aspects, the method is used to improve cardiac function in subjects with DMD. In some aspects, cardiac function is improved by, e.g., increased ejection fraction (EF); increased fractional shortening (FS); decreased left ventricular internal diameter, disastolic (LVIDd); decreased left ventricular end-systolic diameter (LVESD); and/or maintaining or decreasing serum troponin blood levels. In any of the methods of the present disclosure, the subject may be suffering from a muscular dystrophy, such as DMD, or any other dystrophin-associated muscular dystrophy.

In other aspects of any of the methods of the present disclosure described herein, the serum CK level in the subject is decreased after administration of the rAAV as compared to the serum CK level before administration of the rAAV by a percentage level selected from the group consisting of:
a) at least 78% by 90, 180, or 270 days after the administration;
b) at least 46, 55, 56, 57, 58, 59, 60, 61, 62, 63, 64, 65, 66, 67, 68, 69, 70 or 85% by 270 days after the administration;
c) at least 72, 73, 74, or 95% by 180 days after the administration;
d) at least 87, 88, 93 or 95% by 90 days after the administration;
e) at least 70% by 270 days after the administration;
f) 70 to 95% by 90, 180, or 270 days after the administration;
g) at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In another aspect, the present disclosure provides for compositions for treating a muscular dystrophy in a human subject in need, wherein the composition comprises a dose of recombinant adeno-associated virus (rAAV) rAAV.MHCK7.microdystrophin, wherein composition is formulated for a systemic route of administration and the dose of the rAAV is about 1×10¹⁴ vg/kg to about 4×10¹⁴ vg/kg. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

For example, the composition of the present disclosure comprises a dose of rAAV of about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In one aspect, the compositions of the present disclosure are formulated for intravenous administration and comprise a dose of rAAV that is about 2.0×10¹⁴ vg/kg. In another aspect, the compositions of the present disclosure are formulated for intravenous administration and comprise a dose of rAAV that is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In another aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In any of the compositions of the present disclosure, the dose of rAAV is delivered in about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/kg, or about 10 mL/kg to 15 mL/kg or 10 mL/kg to about 20 mL/kg. In a particular aspect, the composition comprises a dose of the rAAV delivered in about 10 mL/kg. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In another aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

The compositions of the present disclosure are formulated for administration by injection, infusion or implantation. For example, the compositions are formulated for administration by infusion over approximately one hour. In addition, the compositions of the present disclosure are formulated for intravenous administration through a peripheral limb vein such as a peripheral arm vein or a peripheral leg vein. Alternatively, the infusion may be administered over approximately 30 minutes, or approximately 1.5 hours, or approximately 2 hours, or approximately 2.5 hours or approximately 3 hours.

Any of the compositions of the present disclosure comprise a rAAV comprising the human micro-dystrophin nucleotide sequence of SEQ ID NO: 1 and the MHCK7 promoter sequence of SEQ ID NO: 2 or SEQ ID NO:7 or a rAAV vector comprising the AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In particular, the compositions of the present disclosure are used to treat Duchenne muscular dystrophy or Becker's muscular dystrophy. For example, the present disclosure provides for compositions for treating Duchenne muscular dystrophy or Becker's muscular dystrophy in a human subject in need thereof wherein the composition comprises a dose of recombinant adeno-associated virus (rAAV) rAAV.MHCK7.microdystrophin, wherein the composition is formulated for administration by intravenous infusion over approximately one hour and the dose of the rAAV administered is about 2×10¹⁴ vg/kg, and wherein the rAAV comprises the AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In another aspect, the present disclosure also provides a composition comprising rAAV for reducing fibrosis in a subject in need thereof. In addition, the present disclosure provides a composition comprising a rAAV vectors for preventing fibrosis in a subject suffering from a muscular dystrophy.

The present disclosure also provides for compositions comprising rAAV for increasing muscular force and/or muscle mass in a subject suffering from a muscular dystrophy. In a further aspect, the present disclosure provides for compositions comprising any of the rAAV of the present disclosure for treatment of muscular dystrophy.

In other aspects of any of the compositions of the present disclosure, after administration of said composition to a human subject in need of treatment for muscular dystrophy, the serum CK level in the subject is decreased as compared to the serum CK level before administration of the composition by a percentage level selected from the group consisting of:
a) at least 78% by 90, 180, or 270 days after the administration;
b) at least 46, 55, 70, or 85% by 270 days after the administration;
c) at least 72, 73, 74, or 95% by 180 days after the administration;
d) at least 87, 99, 93 or 95% by 90 days after the administration;
e) at least 70% by 270 days after the administration;
f) 70 to 95% by 90, 180, or 270 days after the administration;
g) at least 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In another aspect, the present disclosure provides for use of a dose of recombinant adeno-associated virus (rAAV) rAAV.MHCK7.microdystrophin for the preparation of a medicament for the treatment of muscular dystrophy in a human subject in need thereof, wherein the medicament is formulated for a systemic route of administration and the dose of the rAAV is about 1×10¹⁴ vg/kg to about 4×10¹⁴ vg/kg. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

For example, the medicament comprises a dose of rAAV of about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In one aspect, the medicaments of the present disclosure are formulated for systemic administration of a dose of rAAV wherein the systemic route of administration is an intravenous route and the dose of the rAAV administered is about 2.0×10¹⁴ vg/kg. In another aspect, the medicament of the present disclosure is formulated for systemic administration of a dose of rAAV wherein the systemic route of administration is an intravenous route and the dose of the rAAV is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵ vg/kg. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In any of the uses of the present disclosure, the medicament comprises a dose of rAAV in about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/k, or about 10 mL/kg to 15 mL/kg or 10 mL/kg to about 20 mL/kg. In a particular aspect, the dose or the rAAV is in about 10 mL/kg. In one aspect, the rAAV is AAVrh74.MHCK7.microdystrophin. In one aspect, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one aspect, the rAAV is AAVrh74.MCK.microdystrophin. In one aspect, the AAVrh74.MCK.microdystrophin is the AAVrh74.MCK.microdystrophin of nucleotides 56-4820 of SEQ ID NO: 5.

In any of the uses of the present disclosure, the medicament is formulated for administration by injection, infusion or implantation. For example, the medicament is formulated for administration by infusion over approximately one hour. In addition, the medicament is formulated for intravenous administration through a peripheral limb vein, such as a peripheral arm vein or a peripheral leg vein. Alternatively, the infusion may be administered over approximately 30 minutes, or approximately 1.5 hours, or approximately 2 hours, or approximately 2.5 hours or approximately 3 hours.

In any of the uses of the present disclosure, the medicament comprises an rAAV comprising the human micro-dystrophin nucleotide sequence of SEQ ID NO: 1 and the MHCK7 promoter sequence of SEQ ID NO: 2 or SEQ ID NO:7 or the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

A particular use of the present disclosure is for preparation of a medicament for the treatment of Duchenne muscular dystrophy or Becker's muscular dystrophy. For example, the present disclosure provides for use of a dose of recombinant adeno-associated virus (rAAV) rAAV.MHCK7.microdystrophin for the preparation of a medicament for treating Duchenne muscular dystrophy or Becker's muscular dystrophy in a human subject in need thereof, wherein the medicament is formulated for administration by intravenous infusion over approximately one hour and the dose of the rAAV administered is about 2×10¹⁴ vg/kg, and wherein the rAAV comprises the AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In a further aspect, the present disclosure provides for use of a rAAV for preparation of a medicament for reducing fibrosis in a subject in need thereof. For example, the subject in need can be suffering from a muscular dystrophy, such as DMD or any other dystrophin associated muscular dystrophy.

In another aspect, the present disclosure provides for use of a rAAV for the preparation of a medicament to prevent fibrosis in a subject suffering from a muscular dystrophy.

In addition, the present disclosure provides for use of a rAAV for the preparation of a medicament to increase muscular strength and/or muscle mass in a subject suffering from muscular dystrophy.

The present disclosure also provides for use of the rAAV for the preparation of a medicament for treatment of muscular dystrophy.

The present disclosure provides for use of a rAAV vector comprising the human micro-dystrophin nucleotide sequence of SEQ ID NO: 1 and the MHCK7 promoter nucleotide sequence of SEQ ID NO: 2 or SEQ ID NO:7 for preparation of a medicament for the treatment of a muscular dystrophy or a rAAV vector comprising the AAVrf74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for treatment of muscular dystrophy.

In other aspects of any of the uses of the present disclosure, the serum CK level in the subject is decreased after administration of the rAAV to the subject as compared to the serum CK level before administration of the rAAV by a percentage level selected from the group consisting of:
a) at least 78% by 90, 180, or 270 days after the administration;
b) at least 46, 55, 70, or 95% by 270 days after the administration;
c) at least 72, 73, 74, or 95% by 180 days after the administration;
d) at least 87, 88, 93 or 95% by 90 days after the administration;
e) at least 70% by 270 days after the administration;
f) 70 to 95% by 90, 180, or 270 days after the administration;
g) at least 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In any of the compositions for treating a muscular dystrophy or the uses of a medicament for treating a muscular dystrophy, the level of micro-dystrophin gene expression in a cell of the subject is increased after administration of the composition or medicament. Expression of the micro-dystrophin gene in the cell is detected by measuring the micro-dystrophin protein level by Western blot in muscle biopsied before and after administration of the composition or medicament. In particular, the level of micro-dystrophin protein is increased by at least about 70% to at least about 80%, or at least about 70% to at least about 90%, or at least about 80% to at least about 90% after administration of the composition or medicament compared to the level of micro-dystrophin before administration of the composition or medicament. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of the composition compared to the level of micro-dystrophin before administration of the composition or medicament.

In addition, expression of the micro-dystrophin gene in the cell is detected by measuring the micro-dystrophin protein level by immunohistochemistry in muscle biopsies before and after administration of the composition or medicament. The level of micro-dystrophin protein is increased by at least about 70% to at least about 80%, or at least about 70% to at least about 90%, or at least about 80% to at least about 90% after administration of rAAV compared to the level of micro-dystrophin before administration of the composition or medicament. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of the composition or medicament compared to the level of micro-dystrophin before administration of the composition or medicament.

In any of the compositions for treating a muscular dystrophy, the serum CK level in the subject is decreased after administration of the rAAV as compared to serum CK level before administration of the composition or medicament. For example, the serum CK level in the subject is decreased by about 65% to about 90% or about 65% to about 95% or about 75% to about 90% or about 80% to about 90% or about 85% to about 95% or about 87% to about 95% or about 87% to about 90% by 60 days after administration of the composition or medicament as compared to the serum CK level before administration of the composition or medicament. In particular, in any of the compositions for treating a muscular dystrophy of the present disclosure, the serum CK level in the subject is decreased by about 87% by 60 days after administration of the composition or medicament as compared to the serum CK level before administration of the composition or medicament or in any of the compositions for treating a muscular dystrophy or the uses of a medicament for treating a muscular dystrophy of the present disclosure, the serum CK level in the subject is decreased by about 72% by 60 days after administration of the composition or medicament as compared to the serum CK level before administration of the composition or medicament, or in any of the compositions for treating a muscular dystrophy of the present disclosure, the serum CK level in the subject is decreased by about 73% by 60 days after administration of the composition or medicament as compared to the serum CK level before administration of the composition or medicament, or in any of the compositions for treating a muscular dystrophy or the uses of a medicament for treating a muscular dystrophy of the present disclosure, the serum CK level in the subject is decreased by about 78% by 60 days after administration of the composition or medicament as compared to the serum CK level before administration of the composition or in any of the compositions for treating a muscular dystrophy or the uses of a medicament for treating a muscular dystrophy of the present disclosure, the serum CK level in the subject is decreased by about 95% by 60 days after administration of the composition or medicament as compared to the serum CK level before administration of the composition or medicament. In any of the composition s for treating a muscular dystrophy or the uses of a medicament for treating a muscular dystrophy, the number of micro-dystrophin positive fibers in the muscle tissue of the subject is increased after administration of the composition or medicament as compared to the number of micro-dystrophin positive fibers before administration of the composition or medicament. For example, the number of micro-dystrophin positive fibers is detected by measuring the micro-dystrophin protein level by Western blot or immunohistochemistry on muscle biopsies before and after administration of the composition or medicament.

In any of the compositions for treating a muscular dystrophy or the uses of a medicament for treating a muscular dystrophy, administration of the composition or medicament upregulates expression of DAPC proteins such as alpha-sarcoglycan or beta-sarcoglycan. For example, the level of alpha-sarcoglycan in the subject is increased after administration of the composition or medicament as compared to the level of alpha-sarcoglycan before administration of the composition or medicament. In addition, the level of beta-sarcoglycan in the subject is increased after administration of the composition or medicament as compared to the level of the beta-sarcoglycan before administration of the composition or medicament. The level of alpha-sarcoglycan or beta-sarcoglycan is detected by measuring the alpha-sarcoglycan or beta-sarcoglycan protein level by Western blot or immunohistochemistry on muscle biopsies before and after administration of the composition or medicament.

In any of the compositions for treating a muscular dystrophy or the uses of a medicament for treating a muscular dystrophy, disease progression in the subject is delayed after administration of the composition or medicament as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, after administration of any of the compositions for treating a muscular dystrophy or the uses of a medicament for treating a muscular dystrophy, the subject has at least a 1.5, 2.0, 2.5, 2.6, 2.7, 2.8, 2.9, 3.0, 3.1, 3.2, 3.3, 3.4, 3.5, 4.0, 4.5, 5.0, 5.5, 6.0, 6.1, 6.2, 6.3, 6.4, or a 6.5 point improvement in NSAA score at least 270 days after administration of the composition or medicament as compared to NSAA score before administration of the rAAV. Further, in any of the methods, the subject has at least about 0.8 second improvement in time to rise at least 270 days after administration of the composition or medicament as compared to time to rise before administration of the composition or medicament. In addition, in any of the methods or uses of the present disclosure, the subject has at least about 1.2 second improvement in time to ascend 4 steps test at least 270 days after administration of the composition or medicament as compared to time to ascend 4 steps test before administration of the composition or medicament. In addition, in any of the methods or uses of the present disclosure, the subject has at least about 7 second improvement in the 100 m timed test at least 270 days after administration of the composition or medicament as compared to the 100 m timed test before administration of the composition or medicament.

In another aspect, the present disclosure provides for compositions for expressing micro-dystrophin gene in a patient cell comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6. In a further aspect, the present disclosure provides for use of a dose of a AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6 for the preparation of a medicament for expressing the micro-dystrophin gene in a patient cell. For example, expression of the micro-dystrophin gene in the patient cell is detected by measuring the micro-dystrophin protein level by Western blot or immunohistochemistry in muscle biopsies before and after administration of the rAAV.MHCK7.microdystrophin construct. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In a further aspect, the present disclosure provides for compositions for decreasing serum CK levels in a patient in need thereof, the composition comprising the AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In addition, the present disclosure provides for use of a dose of AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for decreasing serum CK levels in a patient in need thereof. For example, the serum CK level in the patient is decreased by at least about 65% to about 90% or about 65% to about 95% or about 75% to about 90% or about 80% to about 90% or about 85% to about 95% or about 87% to about 95% or about 87% to about 90% by 60 days after administration of the composition or medicament as compared to the serum CK level before administration of the composition or medicament. In particular, the serum CK level in the subject is decreased by about 87% by 60 days after administration of the composition or medicament as compared to the serum CK level before administration of the composition or medicament, or decreased by about 72% by 60 days after administration of the composition or medicament as compared to the serum CK level before administration of the composition or medicament, or decreased by about 73% by 60 days after administration of the composition or medicament as compared to the serum CK level before administration of the composition or medicament, or decreased by about 78% by 60 days after administration of the composition or medicament as compared to the serum CK level before administration of the composition or medicament, or decreased by about 95% by 60 days after administration of the composition or medicament as compared to the serum CK level before administration of the composition or medicament.

The present disclosure also provides for compositions for increasing micro-dystrophin positive fibers in a patient muscle tissue comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In addition, the present disclosure provides for use of a dose of AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing micro-dystrophin positive fibers in a patient muscle tissue. For example, the number of micro-dystrophin positive fibers is detected by measuring the dystrophin protein level by Western blot or immunohistochemistry on muscle biopsies before and after administration of the composition or medicament. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In another aspect, the present disclosure provides for compositions for increasing the expression of alpha-sarcoglycan in a patient in need thereof comprising the AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. The present disclosure also provides for use of a dose of AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing the expression of alpha-sarcoglycan in a patient in need thereof. For example, the level of alpha-sarcoglycan is detected by measuring the alpha-sarcoglycan protein level by Western blot or immunohistochemistry on muscle biopsies before and after administration of the composition or medicament.

In addition, the present disclosure provides for compositions for increasing the expression of beta-sarcoglycan in a patient in need thereof comprising the AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. The present disclosure also provides for use of the AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing the expression of beta-sarcoglycan in a patient in need thereof. For example, the level of beta-sarcoglycan is detected by measuring the beta-sarcoglycan protein level by Western blot or immunohistochemistry on muscle biopsies before and after administration of the composition or medicament.

The present disclosure also provides for use of a dose of AAVrh74.MHCK7.microdystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for treating a patient with Duchenne muscular dystrophy or Becker muscular dystrophy, such that administration of the medicament results in disease progression in the patient is delayed as measured by any of: the six minute walk test, time to rise, ascend 4 steps, ascend and descend 4 steps, North Star Ambulatory Assessment (NSAA), 10 meter timed test, 100 meter timed test, hand held dynamometry (HID), Timed Up and Go, and/or Gross Motor Subtest Scaled (Bayley-III) score.

For example, the subject has at least a 1.5, 2.0, 2.5, 2.6, 2.7, 2.8, 2.9, 3.0, 3.1, 3.2, 3.3, 3.4, 3.5, 4.0, 4.5, 5.0, 5.5, 6.0, 6.1, 6.2, 6.3, 6.4, or a 6.5 point improvement in NSAA score at least 270 days after administration of the composition or medicament as compared to NSAA score before administration of the composition or medicament. Further, the subject has at least about 0.8 second improvement in time to rise at least 270 days after administration of the composition or medicament as compared to time to rise before administration of the composition or medicament. In addition, the subject has at least about 1.2 second improvement in time to ascend 4 steps test at least 270 days after administration of the composition or medicament as compared to time to ascend 4 steps test before administration of the composition or medicament. In addition, the subject has at least about 7 second improvement in the 100 m timed test at least 270 days after administration of the composition or medicament as compared to the 100 m timed test before administration of the composition or medicament.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The following Examples are provided by way of illustration and not limitation. Described numerical ranges are inclusive of each integer value within each range and inclusive of the lowest and highest stated integer.

### EXAMPLES

### Example 1

### A) Generation of the AAVrh74.MHCK7.Microdystrophin Construct

The AAVrh74.MHCK7.micro-dystrophin plasmid contains a human micro-dystrophin cDNA expression cassette flanked by AAV2 inverted terminal repeat sequences (ITR) (see FIG. 1). The micro-dystrophin construct was characterized by an in-frame rod deletion (R4-R23), while hinges 1, 2 and 4 and cysteine rich domain remain producing a 138 kDa protein. The expression of the micro-dystrophin protein (3579 bp) was guided by a MHCK7 promoter (792 bp). The plasmid was constructed from the rAAV.MCK.micro-dystrophin plasmid by removing the MCK promoter and inserting the MHCK7 promoter. After the core promoter, the 53 bp endogenous mouse MCK Exon1 (untranslated) is present for efficient transcription initiation, followed by the SV40 late 16S/19S splice signals (150 bp) and a small 5′UTR (61 bp). The intron and 5′ UTR are derived from plasmid pCMVβ (Clontech). The micro-dystrophin cassette had a consensus Kozak immediately in front of the ATG start and a small 53 bp synthetic polyA signal for mRNA termination. The human micro-dystrophin cassette contained the (R4-R23/Δ71-78) domains as previously described by Harper et al. (Nature Medicine 8, 253-261 (2002)). The complementary DNA was codon optimized for human usage and synthesized by GenScript (Piscataway, NJ) (Mol Ther 18, 109-117 (2010)). The only viral sequences included in this vector were the inverted terminal repeats of AAV2, which are required for both viral DNA replication and packaging. The micro-dystrophin cassette has a small 53 bp synthetic polyA signal for mRNA termination.

Previous studies have validated cardiac expression using MHCK7 promoter (Salva et al., Mol Ther 15, 320-329 (2007) and AAVrh74 achieving skeletal, diaphragm, and cardiac muscle expression (Sondergaard et al. Annals of Clinical and Transl Neurology 2, 256-270 (2015)), The sequence of the construct of FIG. 1 was encapsidated into AAVrh.74 virions. The molecular clone of the AAVrh.74 serotype was cloned from a rhesus macaque lymph node and is discussed in in Rodino-Klapac et al. Journal of Translational Medicine 5, 45 (2007).

Table 1 shows the molecular features of the plasmid AAVrh74.MHCK7.micro-dystrophin (SEQ ID NO: 3)

TABLE 1
Molecular features of plasmid rAAV.MHCK7.micro-dystrophin

| TYPE | START | END | NAME | DESCRIPTION |
|---|---|---|---|---|
| REGION | 55 | 182 | 5′ ITR | Wild-type AAV2 inverted terminal repeat |
| REGION | 2 44 | 1035 | MHCK7 | Mouse myosin heavy chain complex-E box muscle |
| | | | | creatine kinase fusion enhancer/promoter |
| REGION | 1045 | 1194 | Chimeric | 5′ donor site from human β-globin gene and |
| | | | intron | the branchpoint and 3′ splice acceptor site |
| | | | | from IgG heavy chain variable region |
| GENE | 1205 | 4783 | huDys cDNA | Human micro-dystrophin cDNA |
| REGION | 4786 | 4838 | PolyA | Synthetic PolyA |
| REGION | 4894 | 5021 | 3′ ITR | Wild-type AAV2 inverted terminal repeat |
| GENE | 6760 | 7619 | AmpR | β-lactamase gene |
| REGION | 7823 | 8442 | Ori | Plasmid origin of replication |

B) Generation of the AAVrh74.MHCK7.Micro-Dystrophin Construct from and Plasmid Encoding Kanamycin (Kan) Resistance

Cloning of MHCK7.pDys.KAN was achieved by isolating the MHCK7.pDys fragment from an MHCK7.pDys.AMP plasmid and the Kanamycin Backbone, and annealing them using the NEBuilder cloning workflow. The MHCK7.pDys fragment was isolated via restriction enzyme digestion with SnaBI. The digestion was performed in a 50 μL total reaction in 1× CutSmart Buffer (NEB) and 1 μL SnaBI, at 37° C. for 1 hour. The resulting fragment was isolated via electrophoresis using a 1% Agarose gel, running at 105 volts for 1.5 hours. The band corresponding to the MHCK7.pDys insert was cut out and purified using a gel purification kit (Macherey-Nagel). The resulting fragment had a DNA concentration of 10 ng/μL. The Kan backbone fragment was isolated via XbaI restriction enzyme digestion in a 50 μL reaction with 1× CutSmart Buffer (NEB) and 1 μL XbaI, at 37° C. for 1 hour. The resulting fragment was isolated via electrophoresis using a 1% Agarose gel, running at 105 volts for 1.5 hours. The band corresponding to the Kan Backbone was cut out and purified via gel purification kit (Macherey-Nagel). The resulting fragment had a DNA concentration of 8.1 ng/μL. The two fragments were annealed using the NEB Builder cloning workflow, which has the ability to join two fragments with overlapping sequences. The NEBuilder cloning reaction was performed per manufacturer protocol at 50° C. for 15 minutes, using a 1:1 ratio of MHCK7.μDys to Kanamycin backbone in 1×NEBuilder HiFi DNA Assembly Master Mix for a total reaction volume of 20 μL. The resulting clone was transformed into NEB® Stable Competent E. coli (C3040) by adding 2.5 μL cloning product to the cells followed by 30 minutes on ice, then 30 seconds at 42° C. and an additional 5 minutes on ice. After transformation, 950 μL of outgrowth media was added to the cells and allowed to grow at 30° C. for 1.5 hours, shaking at 225 rpm. Following outgrowth, 450 μL of these cells was plated on a 50 μg/mL kanamycin LB agar plate and incubated overnight at 30° C. in a dry incubator. A colony was picked from this plate and grown up overnight in LB containing 50 μg/mL kanamycin. DNA was isolated from 3 mL of this culture using QIAprep® Spin Miniprep Kit (Qiagen). This DNA was used to confirm the cloning product. The cloning product was confirmed via restriction enzyme digestion with PmeI, MscI, and SmaI followed by gel electrophoresis. The cloning product was additionally confirmed via sequencing. The resultant plasmid is set forth in SEQ ID NO:8, and shown in FIGS. 14 and 15. The sequence of construct of FIG. 13 which corresponds to that of SEQ ID NO:9, and nucleotides 1-4977 of SEQ ID NO: 8, was encapsidated into AAVrh.74 virions as described above.

### Example 2

### Systemic Gene Delivery Clinical Trial for Duchenne Muscular Dystrophy

This was a single-dose controlled trial using the rAAVrh74.MHCK7.micro-dystrophin of SEQ ID NO: 3, nucleotides 55-5021, for DMD subjects. Cohort A included six subjects of ages 3 months to 3 years, and Cohort B included six subjects of ages 4 years to 7 years old. All subjects received intravenous micro-dystrophin vector (2×10¹⁴ vg/kg in 10 mL/kg). The rAAVrh74.MHCK7.micro-dystrophin was formulated in a buffer containing 20 mM Tris (pH 8.0), 1 mM magnesium chloride (MgCl₂), 200 mM sodium chloride (NaCl), and 0.001% poloxamer 188.

In the study, the rAAVrh74.MHCK7.micro-dystrophin was infused via peripheral arm vein so that it can reach all the muscles in the body. Six DMD subjects ages 3 months to 3 years in Cohort A, and six DMD subjects ages 4 years to age 7 years in Cohort B, were enrolled. All subjects received intravenous micro-dystrophin vector (2×10¹⁴ vg/kg in 10 mL/kg). The encapsulated vector genome titer for the administered dose was determined using quantitative PCR using a Prism 7500 Taqman detector system (PE Applied Biosystems) with primers directed against the MHCK7 promoter compared to a supercoiled DNA plasmid standard (Pozsgai et al., Mol. Ther. 25(4):855-869 (2017)).

Subjects received infusions over 1 hour in the Pediatric Intensive Care Unit (PICU) at Nationwide Children's Hospital. Before the gene therapy, a muscle biopsy was performed at the screening visit. Subjects will have a second muscle biopsy to determine if the gene allowed for replacement of the missing dystrophin protein at 90 days post-delivery. After the gene transfer, patients were carefully monitored for any side effects of the treatment. This monitoring included blood and urine tests, as well as physical examination during the screening visits and on days 0, 1, 7, 14, 30, 60, 90, and 180, and at months 9, 12, 18, 24, 30 and 36 to make sure that there are no side effects from the gene injection.

The subjects of Cohort A (n=6) were between 3 months to 3 years of age, and received intravenous rAAVrh74.MHCK7.micro-dystrophin vector (2×10¹⁴ vg/kg in 10 mL/kg). One-day prior to gene transfer, subjects in Cohort A were started on prednisone or deflazacort 1 mg/kg and maintained for 30 days while monitoring immune response. If negative at day 30, steroids were weaned over 1 week. If T cell response to AAV or micro-dystrophin was >125 SFC/106 PBMCs, steroids were maintained until levels drop below this threshold.

The subjects of Cohort B (n=6) were between 4 years to 7 years of age, and received intravenous rAAVrh74.MHCK7.micro-dystrophin vector (2×10¹⁴ vg/kg in 10 mL/kg). These subjects were maintained on stable dose of corticosteroids throughout trial but may have been increased for short time if T cell response to AAV or micro-dystrophin was >125 SFC/106 PBMCs.

### Eligibility Criteria

Inclusion criteria for the study were as follows:
Age of enrollment: Cohort A: 3 months to 7 years of age and Cohort B: between 4-7 years of age, inclusive.
Molecular characterization of the DMD gene with frameshift (deletion or duplication), or premature stop codon mutation between exons 18 to 58.
CK elevation >1000 U/L
Cohort A subjects: below average on the Bayley-III motor assessment for gross motor defined as a scaled score of ≤9
Cohort B: Below average on 100 meter timed test defined as <80% predicted
Males of any ethnic group.
Ability to cooperate with motor assessment testing.
Cohort A subjects: No previous treatment with corticosteroids.
Cohort B subjects: Stable dose equivalent of oral corticosteroids for at least 12 weeks prior to screening and the dose is expected to remain constant (except for modifications to accommodate changes in weight) throughout the stud

Exclusion criteria for the study were as follows:
Active viral infection based on clinical observations.
Signs of cardiomyopathy, including echocardiogram with ejection fraction below 40%.
Serological evidence of HIV infection, or Hepatitis B or C infection.
Diagnosis of (or ongoing treatment for) an autoimmune disease.
Abnormal laboratory values considered clinically significant
Concomitant illness or requirement for chronic drug treatment that in the opinion of the PI creates unnecessary risks for gene transfer.
Subjects with AAVrh74 or AAV8 antibody titers >1:400 as determined by ELISA immunoassay.
Medical condition or extenuating circumstance that, in the opinion of the investigator, might compromise the subject's ability to comply with the protocol required testing or procedures or compromise the subject's wellbeing, safety, or clinical interpretability.
Severe infection (e.g., pneumonia, pyelonephritis, or meningitis) within 4 weeks before gene transfer visit (enrollment may be postponed).
Received any investigational medication (other than corticosteroids) or exon skipping medications (including ExonDys 51@), experimental or otherwise, in the last 6 months prior to screening for this study.
Has had any type of gene therapy, cell based therapy (e.g. stem cell transplantation), or CRISPR/Cas9 therapy.
Family does not want to disclose patient's study participation with primary care physician and other medical providers.

### Outcome Measures

The primary outcome measure was safety based on number of participants with adverse events (time frame: 3 years). Adverse effects were monitored and scored for severity and relatedness to the study article.

The secondary outcome measures were as follows:

Gross Motor Subtest Scaled (Bayley-III) score (time frame: screening, Day 30-3 Years): The Gross Motor Scaled Score measured motor development. The Bayley-III Gross Motor Subtest was scored for Cohort A on every follow up visit starting at Day 30 through 3 years. Any subject that was 43-47 months of age, inclusive, at time of screening had the scaled score calculated compared to normative data for 42 month old children. The Bayley-III provided normative data for children 1-42 months of age.

Physical Therapy Assessments The 100 Meter Timed Test (100 m) (time frame: screening, Day 30-3 Years): The 100 m was the primary motor outcome for Cohort B. The 100 Meter Timed Test was an exploratory outcome initiated for Cohort A as soon as the child was 3 years of age.

Physical Therapy Assessments North Star Ambulatory Assessment (NSAA) (time frame: screening, Day 30-3 Years): The North Star Ambulatory Assessment (NSAA) was an exploratory outcome initiated for Cohort A as soon as the child was four years of age and for cohort B. The NSAA measures the quality of ambulation in young boys with Duchenne Muscular Dystrophy.

Physical Therapy Assessments Timed Up and Go modified for children (TUG) (time frame: screening, Day 30-3 Years): Exploratory outcomes for Cohort B included the Timed Up and Go modified for children (TUG).

Physical Therapy Assessments Ascend and Descend of 4 steps (time frame: screening, Day 30-3 Years): Exploratory outcomes for Cohort B will include ascend and descend of 4 steps.

Physical Therapy Assessments Hand Held Dynamometry (HHD) (time frame: screening, Day 30-3 Years): Exploratory outcomes for Cohort B included hand held dynamometry (HHD) for knee extensors and knee flexors, and elbow flexors and elbow extensors.

Micro-dystrophin gene expression quantification by immunofluorescence (time frame: screening, Day 90): Micro-dystrophin gene expression levels were quantified by immunofluorescence and compared in pre and post muscle biopsies.

Micro-dystrophin gene expression quantification by western blot (time frame: screening, Day 90): Micro-dystrophin gene expression levels were quantified by western blot analysis and compared in pre and post muscle biopsies.

A decrease in CK following gene therapy (time frame: 3 years): Decrease in CK levels in circulating blood.

Cardiac magnetic resonance imaging (at 1 year).

### Micro-Dystrophin Gene Expression

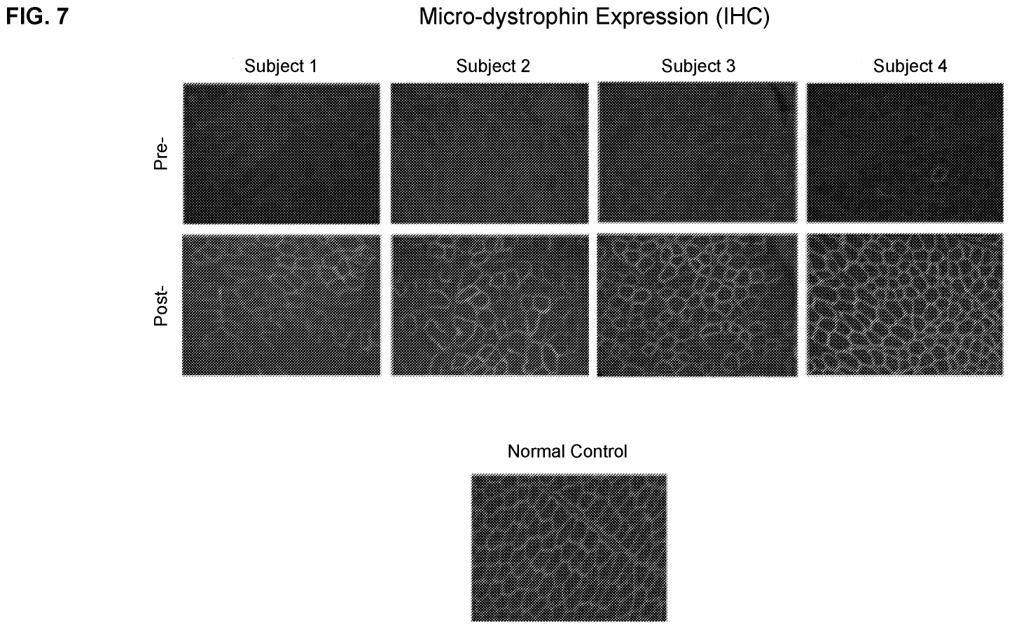

Change from Baseline in micro-dystrophin expression via immunofluorescence (IF) fiber intensity was analyzed and quantitated. As shown in Table 2, Subject 1 (age 5) demonstrated 78% expression of micro-dystrophin protein in the muscle fibers of the gastrocnemius muscle biopsy after administration of the rAAVrh74.MHCK7.micro-dystrophin; Subject 2 (age 4) demonstrated 73.5% expression of micro-dystrophin protein in the muscle fibers of the gastrocnemius muscle biopsy after administration of the rAAVrh74.MHCK7.micro-dystrophin; and Subject 3 (age 6) demonstrated 77.0% expression of micro-dystrophin protein in the muscle fibers of the gastrocnemius muscle biopsy after administration of the rAAVrh74.MHCK7.micro-dystrophin. Subject 4 (age 4) demonstrated 96.2% expression of micro-dystrophin in the muscle fibers of the gastrocnemius muscle biopsy after administration of the rAAVrh74.MHCK7.micro-dystrophin. All patients showed robust expression of transduced micro-dystrophin, which is properly localized to the muscle sarcolemma, as measured by immunohistochemistry (FIG. 7).

TABLE 2

| | | Percentage of Micro- |
|---|---|---|
| Subject | Mean Intensity | Dystrophin-Positive Fibers |
| 1 | 82.0% | 78.0% |
| 2 | 59.0% | 73.5% |
| 3 | 83.0% | 77.0% |
| 4 | 160.0% | 96.2% |
| Mean | 96.0% | 81.2% |

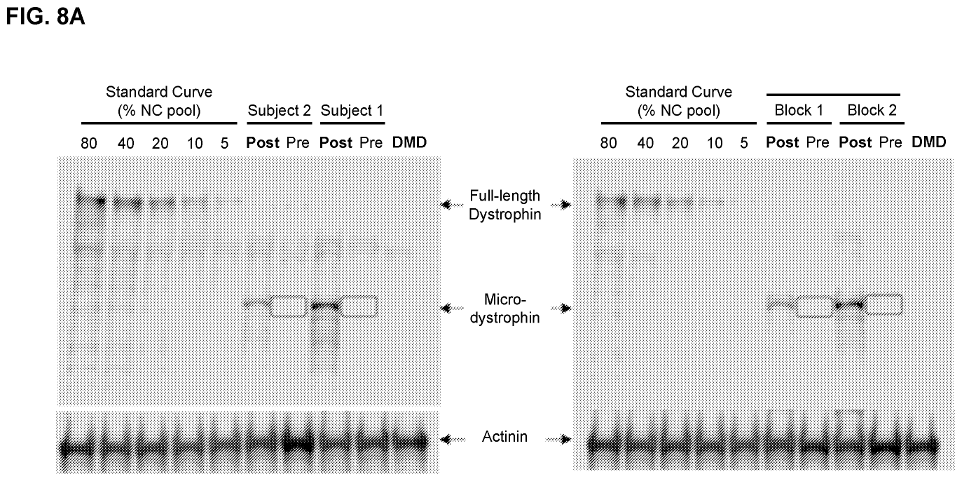

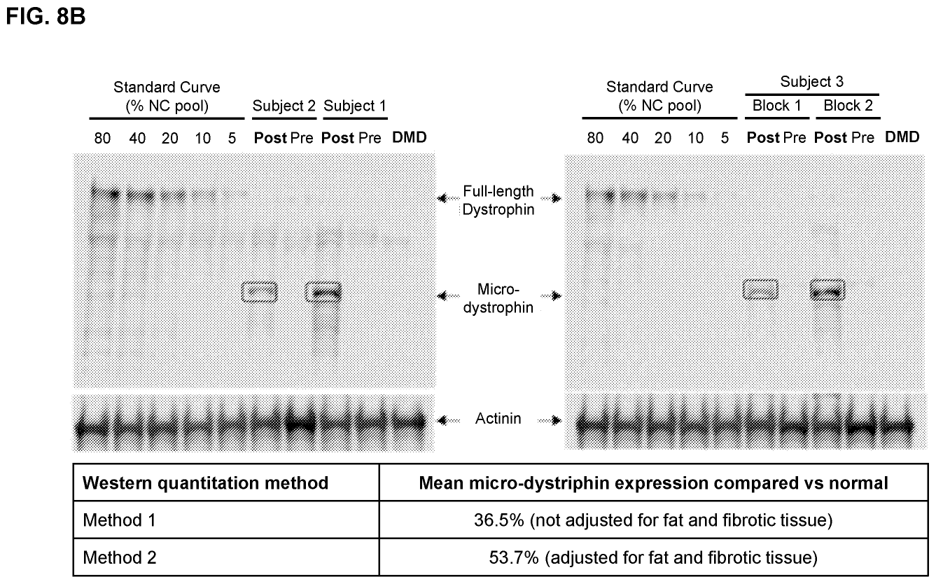

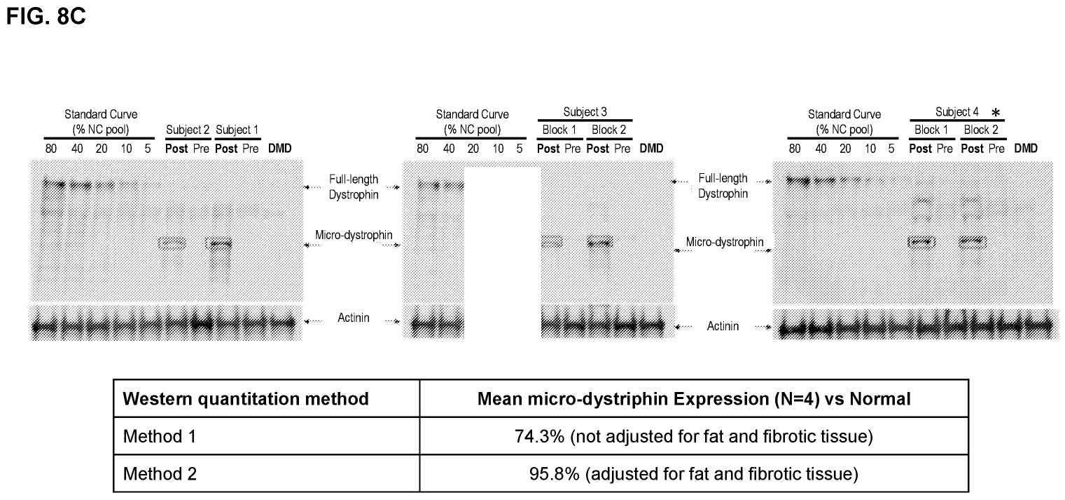

The change in micro-dystrophin gene expression from Baseline to Day 60 was also assessed by quantitating micro-dystrophin protein expression as measured by Western blot of biopsied muscle tissue. As shown in FIGS. 8A and 8B, Western blot analysis detected micro-dystrophin protein expression in Subject 1 (age 5), Subject 2 (age 4), and Subject 3 (age 6). FIG. 8C provides the Western Blot analysis detecting micro-dystrophin protein expression in Subject 4 (age 4). All post-treatment biopsies showed robust levels of micro-dystrophin as measured by Western blot, with a mean for Subjects 1-4 of 74.3 compared to normal utilizing method 1, and 95.8% compared to normal pursuant to method 2 that adjusts for fat and fibrotic tissue.

For each subject, the vector genome copy per nucleus of the muscle fibers were measured. As shown in Table 3, the vector genome copy per nuclease was greater than 1 for each of the subjects after administration of rAAVrh74.MHCK7.micro-dystrophin. One copy of the vector indicates approximately 50% expression of the micro-dystrophin gene. A mean of 1.6 vector copies per cell nucleus was measured in Subjects 1-3, consistent with the high micro-dystrophin expression levels observed. When the values for Subject 4 were included, the mean vectors copies/μg DNA is >105 with a mean of 3.3 vector copies per cell nucleus.

TABLE 3

| Subject | Vector copies/μg DNA | Copies per Nuclei |
|---|---|---|
| 1 | >105 | 1.7 |
| 2 | >105 | 1.3 |
| 3 | >105 | 1.9 |

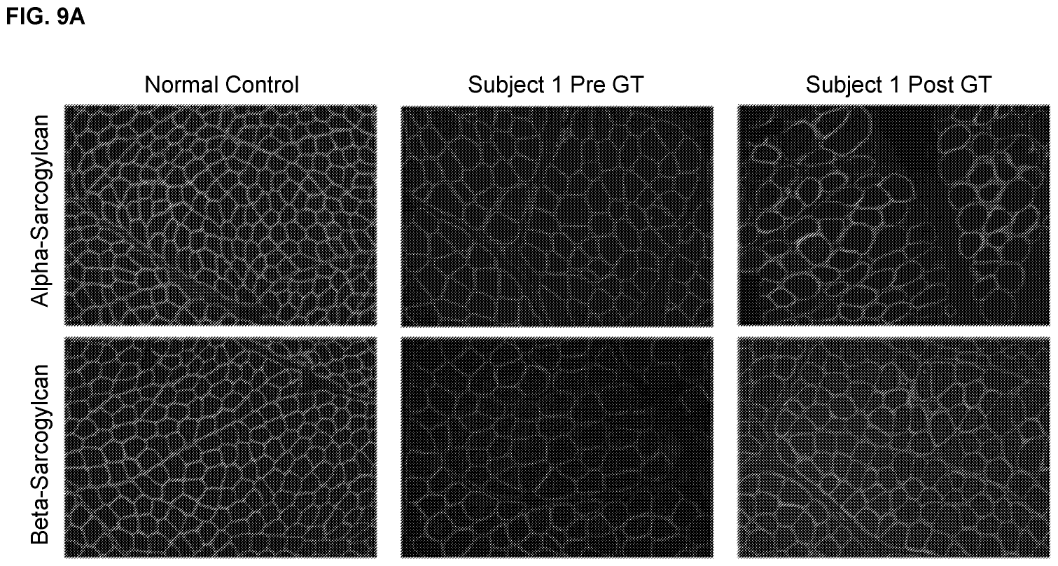

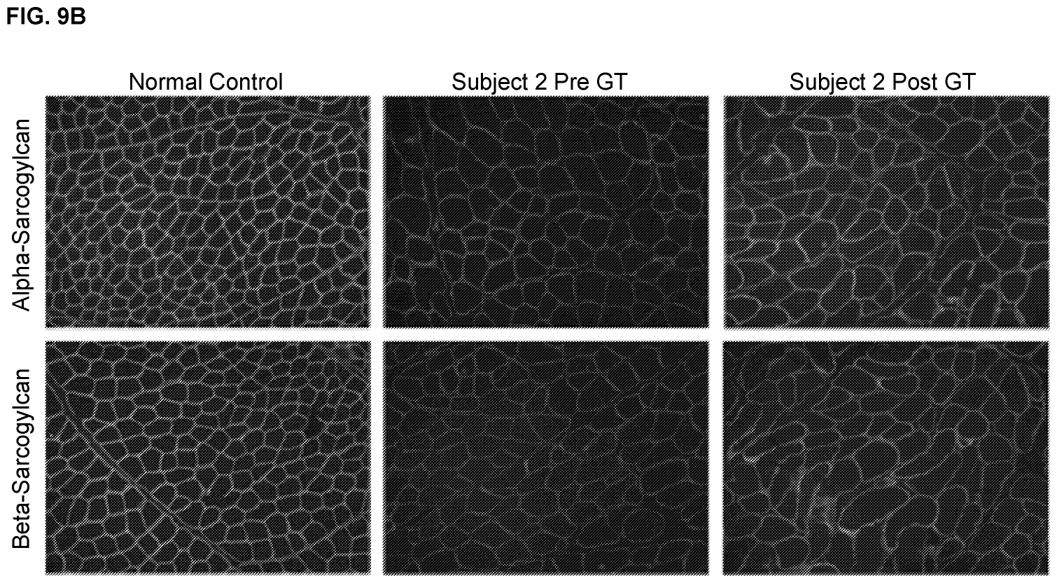

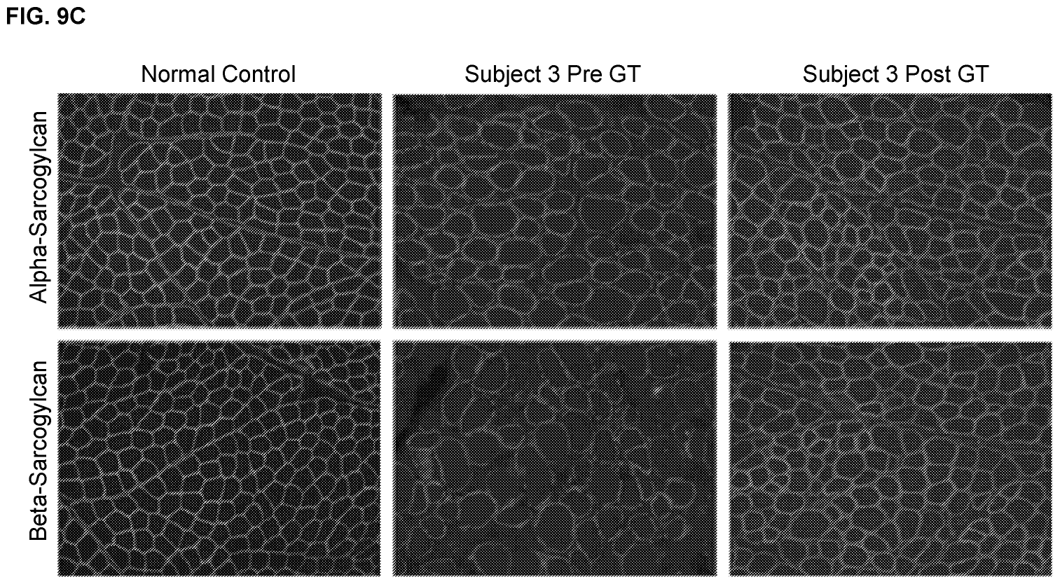

The protein levels of alpha-sarcoglycan and beta-sarcoglycan in muscle biopsy tissue were measured by immunohistochemistry before and after administration of rAAVrh74.MHCK7.micro-dystrophin. Administration of rAAVrh74.MHCK7 also resulted in upregulation of the DAPC proteins in the subjects. As shown in FIG. 9, expression of alpha-sarcoglycan and beta-sarcoglycan in muscle biopsy tissue was increased compared to the level so these proteins in muscle biopsies before administration of rAAVrh74.MHCK7 in Subjects 1 (FIG. 9A), Subject 2 (FIG. 9B), and Subject 3 (FIG. 9C).

### Circulating Serum CK Levels

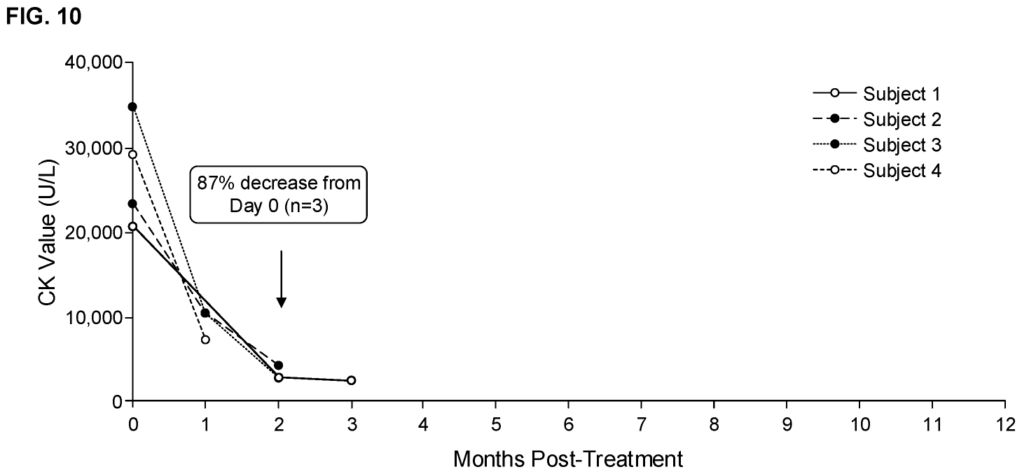

Blood samples were obtained every 30 days after administration intravenous infusion of rAAVrh74.MHCK7.micro-dystrophin vector (2×10¹⁴ vg/kg in 10 mL/kg). CK levels were measured at each visit and compared to the baseline level obtained before administration of the rAAVrh74.MHCK7.micro-dystrophin (Visit Day 0). Baseline serum CK levels (Units/Liter) are provided in Table 4 below. As shown in FIG. 10, the level of circulating serum CK decreased about 87%, 2 months after administration of rAAVrh74.MHCK7.micro-dystrophin. All subjects showed significant decreases of serum creatine kinase (CK) levels, with a mean reduction of CK of over 87% at 2 months' post-treatment (n=3). CK is an enzyme associated with muscle damage and patients with DMD uniformly exhibit high levels of CK. Indeed, significantly elevated CK is often used as a preliminary diagnosis tool for DMD, which is then followed by confirmatory genetic testing.

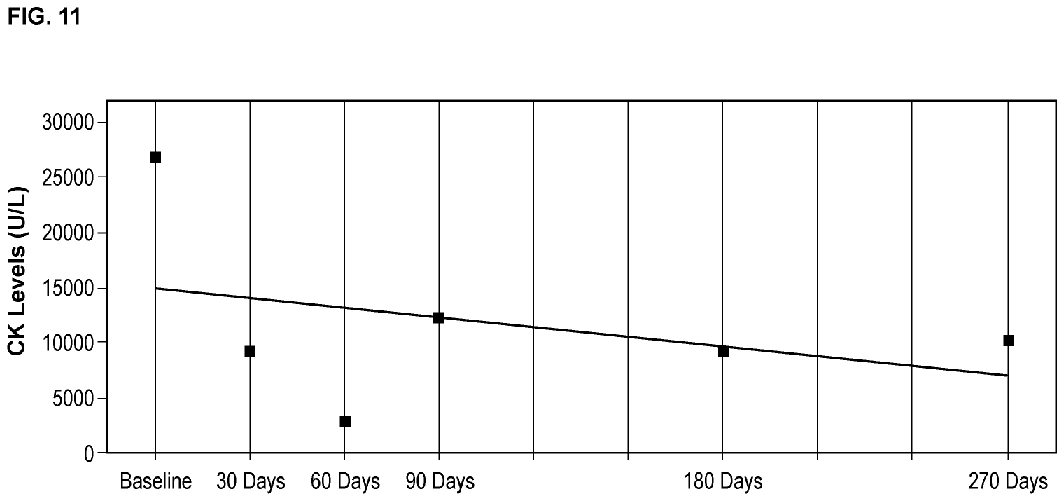

Table 4 and FIG. 10 provide the CK levels for each subject. FIG. 11 provides the mean CK levels over time, and demonstrates that the mean CK levels significantly decrease over time after administration of rAAVrh74.MHCK7.micro-dystrophin. The mean baseline CK level of 27,064 U/L (Mean for Table 4) is decreased by about 63% to a mean of 9,982 U/L (Mean, Day 270, Table 5).

TABLE 4

| | | CK Levels at Baseline |
|---|---|---|
| Subject | Age (years) | Units/Liter (U/L) |
| 1 | 5 | 20691 |
| 2 | 4 | 23414 |
| 3 | 6 | 34942 |
| 4 | 4 | 29210 |

TABLE 5
Change in CK levels from Baseline to Day 270

| Subject | Baseline | Day 30 | Day 60 | Day 90 | Day 180 | Day 270 | Day 360 |
|---|---|---|---|---|---|---|---|
| 1 | | — | 2984 | 2444 | 18476 | 6317 | — |
| 2 | 23414 | 10427 | 4283 | 41920 | 6209 | 10494 | — |
| 3 | 34942 | 10430 | 2966 | 2546 | 9650 | 18855 | 6410 |
| 4 | 29210 | 7215 | 908 | 1382 | 2580 | 4262 | — |

### Efficacy Assessment

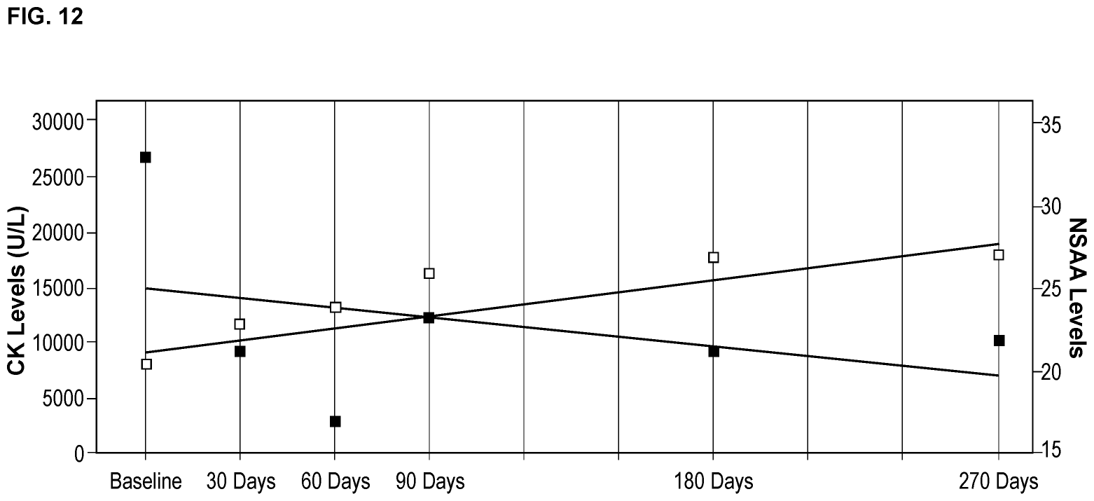

In addition to micro-dystrophin and CK levels, efficacy was measured by the following functional tests: Time to Rise from the floor, Ascend 4 steps, North Star Ambulatory Assessment (NSAA), Time to Rise Test, 4 Stairs Up Test, 10 Meter Timed Test (10 m), and 100 Meter Timed Test (100 m). The data is provided in Tables 6 and 7 below, and this data demonstrates consistent durable improvement at 9 months after administration of rAAVrh74.MHCK7.micro-dystrophin. The NSAA improvement over time is also provided in FIG. 12.

TABLE 6
NSAA Change from Baseline to Day 270

| | | | | | | | Change |
|---|---|---|---|---|---|---|---|
| | | | | | | | from |
| Subject | Baseline | Day 30 | Day 60 | Day 90 | Day 180 | Day 270 | Baseline |
| 1 | 18 | 22 | 24 | 23 | 25 | 26 | 8 |
| 2 | 19 | 21 | 23 | 25 | 27 | 27 | 8 |
| 3 | 26 | 28 | 28 | 30 | 30 | 28 | 2 |
| 4 | 19 | 20 | 20 | 25 | 25 | 27 | 8 |
| Mean | 20.5 | 22.75 | 23.75 | 25.75 | 26.75 | 27 | 6.5 |
| Improvement | | | | | | | |

TABLE 7
Change from Baseline to Day 270

| | Assess- | | Time to | 4 Stairs | 100 m | |
|---|---|---|---|---|---|---|
| Subject | ment | NSAA | Rise | Up | (sec) | 10 m |
| 1 | Baseline | 18 | 3.7 | 3.4 | 49.3 | 5.1 |
| | Day 270 | 26 | 3.0 | 2.3 | 43.2 | 4.3 |
| 2 | Baseline | 19 | 3.0 | 3.8 | 49.9 | 4.3 |
| | Day 180 | 27 | 3.7 | 2.6 | 48.6 | 3.9 |
| | Day 270 | 27 | 3.3 | 2.7 | 50.3 | |
| 3 | Baseline | 26 | 3.9 | 1.9 | 59.3 | 4.7 |
| | Day 180 | 30 | 3.4 | 1.8 | 48.4 | 4.1 |
| | Day 270 | 28 | 2.8 | 1.9 | 50.7 | |
| 4 | Baseline | 19 | 4.1 | 4.8 | 67.2 | 5.4 |
| | Day 90 | 25 | 2.3 | 2.2 | 50.7 | 4.4 |
| | Day 270 | 27 | 2.4 | 2.2 | 49.7 | |
| Average | Change | 6.5 | .8 sec. | 1.2 sec. | 7.95 | 14% |
| | from | point | im- | im- | sec. | im- |
| | Baseline | im- | prove- | prove- | im- | prove- |
| | | prove- | ment | ment | prove- | ment |
| | | ment | | | ment | |

### Safety Assessment

No serious adverse events (SAEs) were observed in the study. Three subjects had elevated gamma-glutamyl transferase (GGT) that resolved with increased steroids within a week and returned to baseline levels. There were no other clinically significant laboratory findings. Patients had transient nausea generally during the first week of therapy coincident with increased steroid dosing. This did not correlate with liver enzyme elevations or any other abnormality.

### Example 3

### Randomized Double-Blind Placebo Controlled Systemic Gene Delivery Phase I/IIa Clinical Trial

This is a randomized double-blind single-dose trial using rAAVrh74.MHCK7.micro-dystrophin for DMD subjects. The study included twenty-four subjects ages 4 to 7 years old. Subjects were randomized to treatment or placebo at the time of enrollment. Twelve subjects received intravenous rAAVrh74.MHCK7.micro-dystrophin vector (2×10¹⁴ vg/kg in approximately 10 mL/kg) and thirteen subjects-received 10 mL/kg placebo (lactated ringers). Placebo subjects will roll over to treatment which will be given in the same manner as the 12 previously treated subjects one year after the last treated subject is dosed. Subjects receive infusions of rAAV carrying micro-dystrophin or lactated ringers over approximately 1 hour. Pre and post-treatment (90 Day) needle muscle biopsies are done on gastrocnemius muscles.

The primary objective of this study is the assessment of the safety of intravenous administration of rAAVrh74.MHCK7.micro-dystrophin for DMD subjects via peripheral limb vein. Safety endpoints are assessed by changes in hematology, serum chemistry, urinalysis, immunologic response to rAAVrh74 and micro-dystrophin, and reported history and observations of symptoms. Dystrophin gene expression serves as a primary outcome measure along with safety. Quantification is carried out using validated immunofluorescence and immunoblot assays. A decrease in CK following gene therapy serves as a secondary outcome. Efficacy is measured by the following functional tests: Time to Rise, Ascend 4 steps, North Star Ambulatory Assessment (NSAA), 10 Meter Timed Test (10 ml), 100 Meter Timed Test (100 m). Exploratory measures include hand-held dynamometry (HHD) for knee extensors and knee flexors, and elbow flexors and elbow extensors.

Inclusion criteria for the study are as follows:
Age of enrollment: between 4-7 years of age, inclusive.
Molecular characterization of the DMD gene with frameshift (deletion or duplication), or premature stop codon mutation between exons 18 to 58.
Indication of symptomatic muscular dystrophy: CK elevation >1000 U/L and Below mean percent predicted time on 100 meter walk test
Males of any ethnic group will be eligible.
Ability to cooperate with motor assessment testing.
Stable dose equivalent of oral corticosteroids for at least 12 weeks prior to screening and the dose is expected to remain constant (except for potential modifications to accommodate changes in weight) throughout the study.

Exclusion criteria for the study are as follows:
Active viral infection based on clinical observations.
Signs of cardiomyopathy, including echocardiogram with ejection fraction below 40%.
Serological evidence of HIV infection, or Hepatitis B or C infection.
Diagnosis of (or ongoing treatment for) an autoimmune disease.
Abnormal laboratory values considered clinically significant (GGT >3XULN, bilirubin ≥3.0 mg/dL, creatinine ≥1.8 mg/dL, Hgb <8 or >18 g/Dl; WBC >18,500 per cmm), platelets ≤50,000.
Concomitant illness or requirement for chronic drug treatment that in the opinion of the PI creates unnecessary risks for gene transfer.
Subjects with AAVrh74 or AAV8 antibody titers >1:400 as determined by ELISA immunoassay. If endpoint titer is positive at screening, testing may be repeated prior to exclusion.
Has a medical condition or extenuating circumstance that, in the opinion of the investigator, might compromise the subject's ability to comply with the protocol required testing or procedures or compromise the subject's wellbeing, safety, or clinical interpretability.
Severe infection (e.g., pneumonia, pyelonephritis, or meningitis) within 4 weeks before gene transfer visit (enrollment may be postponed).
Has received any investigational medication (other than corticosteroids) or exon skipping medications (including ExonDys 51@), experimental or otherwise, in the last 6 months prior to screening for this study.
Has had any type of gene therapy, cell based therapy (e.g. stem cell transplantation), or CRISPR/Cas9 therapy.
Family does not want to disclose patient's study participation with primary care physician and other medical providers.

### Efficacy Assessments

Dystrophin gene expression serves as a primary outcome measure along with safety. Quantification is carried out using validated immunofluorescence and immunoblot assays. A decrease in CK following gene therapy serves as a secondary outcome. In addition, efficacy is measured by the following functional tests: Time to Rise from the floor, Ascend 4 steps, North Star Ambulatory Assessment (NSAA), 10 Meter Timed Test (10 m), 100 Meter Timed Test (100 m)]. Exploratory measures include hand-held dynamometry (HHD) for knee extensors and knee flexors, and elbow flexors and elbow extensors.

Muscle biopsies with ultrasound guidance are used to quantify transgene expression comparing baseline to Day 90. The biopsies are carried out on the same muscle as the original biopsies but on the opposite leg. One year after all subjects have been dosed, placebo crossover subjects will restart the study timeline at visit 1. Placebo subjects will not have the following performed at the second baseline screening: Cardiac MRI and Muscle Biopsy. Placebo subjects undergo a muscle biopsy at Day 90 (total of 3 muscle biopsies). Frozen sections are stained for dystrophin using indirect immunofluorescence (IF). Full slide scanning is performed and micro-dystrophin intensity and percent positive fibers is quantified using validated image scanning and MuscleMap™ analysis algorithm. Muscle morphometrics are performed blinded including fiber size histograms. Blinded frozen muscle biopsy shavings are used to perform quantitative protein analysis for micro-dystrophin using a validated western blot method.

Muscle needle biopsies of the gastroenemius muscle (unless deemed contraindicated in a specific subject by the PI, in which case the PI will select an alternative muscle to biopsy) are used to quantify micro-dystrophin expression.

### Efficacy Analyses

The primary efficacy endpoint is the change from Baseline to Day 90 in the quantity of micro-dystrophin protein expression as measured by Western blot of biopsied muscle tissue. Treatment group differences for the primary efficacy endpoint are assessed with an analysis of covariance (ANCOVA) model with treatment as the fixed factor and baseline value as the covariate. The Wilcoxon rank-sum test is performed as a supportive analysis. Change from Baseline in micro-dystrophin expression via immunofluorescence (IF) fiber intensity is analyzed similarly.

The supportive efficacy endpoints include change from Baseline to each scheduled assessment of Time to Rise from the floor, Ascend 4 steps, NSAA, 10 Meter Timed Test (10 m), 100 Meter Timed Test (100 m), and change in CK. Exploratory measures include HHD for knee extensors and knee flexors, and elbow flexors and elbow extensors. Treatment group differences are assessed with ANCOVA model with treatment as the fixed factor and baseline value as the covariate. The Wilcoxon rank-sum test is performed as a supportive analysis.

### Example 4

The trials and studies described in Examples 2 and 3 above are alternatively carried out utilizing the rAAVrh74.MHCK7.micro-dystrophin construct set forth in SEQ ID NO: 9; as set forth in SEQ ID NO: 8, nucleotides 1-4977; or as set forth in SEQ ID NO: 6, nucleotides 56-5022.

### Example 5

### Generation of the pAAV.MCK.Micro-Dystrophin Construct

The pAAV.MCK.micro-dystrophin plasmid was constructed by inserting the MCK expression cassette driving a codon optimized human micro-dystrophin cDNA sequence into the AAV cloning vector psub201 (Samulski, R. J. et al., J. Virol. 61(10):3096-3101 (1987)). A muscle-specific regulatory element was included in the construct to drive muscle-specific gene expression. This regulatory element comprised the mouse MCK core enhancer (206 bp) fused to the 351 bp MCK core promoter (proximal). After the core promoter, the construct comprises the 53 bp endogenous mouse MCK Exon1 (untranslated) for efficient transcription initiation, followed by the SV40 late 16S/19S splice signals (97 bp) and a small 5′UTR (61 bp). The intron and 5′ UTR was derived from plasmid pCMV8 (Clontech). The micro-dystrophin cassette has a consensus Kozak immediately in front of the ATG start and a small 53 bp synthetic polyA signal for mRNA termination. The human micro-dystrophin cassette contains the (R4-R23/Δ71-78) domains, as previously described by Harper et al., Nat. Med. 8(3):253-61 (2002).

The pAAV.MCK.micro-dystrophin plasmid contained the human micro-dystrophin cDNA expression cassette flanked by AAV2 inverted terminal repeat sequences (ITR) (see FIG. 5). This sequence was encapsidated into AAVrh.74 virions. The molecular clone of the AAVrh.74 serotype was cloned from a rhesus macaque lymph node and is described in Rodino-Klapac et al., J Transl. Med. 5: 45 (2007).

### Example 6

### rAAV Production Using Hybrid Seed Train Expansion

The following process can be used to produce the rAAV constructs described herein.

HEK-293 cells were passaged four times in adherent conditions. Before going into the penultimate expansion culture, the cells were harvested and centrifuged to wash away serum (@300 g for 5 minutes), and resuspended in serum-free growth medium (EXPI293) in suspension shake flasks at a seeding density of 0.5+E6 cells/mL. They were then allowed to grow and expand in numbers for 48-72 hours. The suspension cells were then collected and inoculated in shake flasks or WAVE bags, depending on how many viable cells were required for bioreactor inoculation. At the end of 72 hours, the concentrations of viable cells were determined using cell counting equipment. Then, the necessary volume containing preferred total viable cells was added to the adherent bioreactor containing DMEM and 10% FBS. Additional FBS was appropriately added to account for the addition of serum-free suspension culture volume so that the final FBS concentration was maintained at 10%.

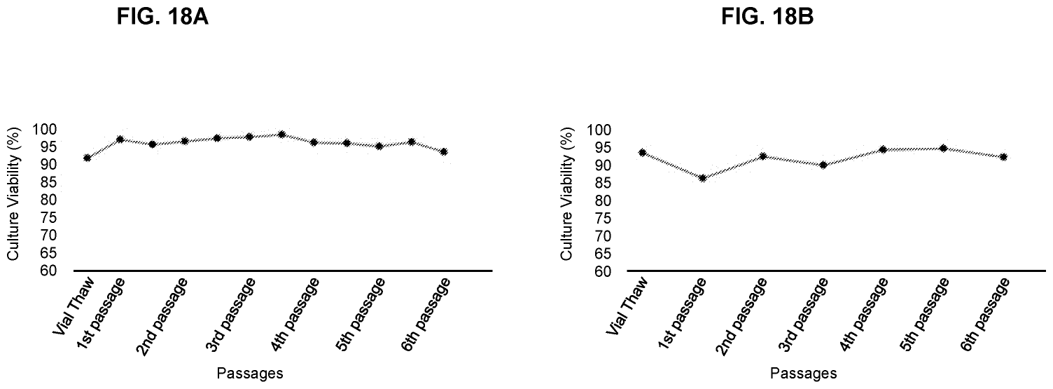

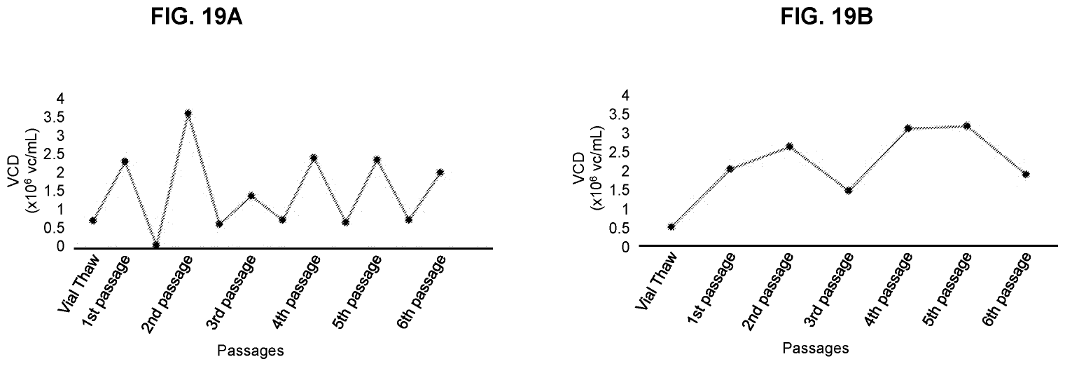

As shown in FIG. 18, the cell viabilities in both seed train and adherent systems were similar. With respect to viable cell density (VCD), in the hybrid seed train system, the VCD after 6^{th} passage was higher than the one after 1^{st} passage (FIG. 19A), which is comparable to the adherent system (FIG. 19B).

Subsequent to inoculating the adherent bioreactor (iCELLis®), the adherent culture is transiently transfected with a transgene plasmid harboring a micro-dystrophin construct described herein, including for example the construct set forth in SEQ ID NO:9 contained in the transgene plasmid of SEQ ID NO:8. In addition to the transgene plasmid, a rep/cap plasmid (AAV2 rep/rh74 cap) and a helper plasmid is included. Following a desired period of growth, rAAV particles are harvested by cell lysis and column chromatography.

In some aspects, the rAAV is produced by a suspension seed process, comprising:
(a) culturing cells with a first growth medium comprising serum in a N-2 container;
(b) removing the cells from the first medium;
(c) inoculating the cells from step (b) into a second medium comprising no serum or serum at a concentration less than the first medium in a N-1 container;
(d) culturing the cells in the N-1 container under suspension conditions; and
(e) inoculating a third medium in a bioreactor with the cells from step (d).

In some aspects, the suspension seed process further comprises:
(f) transfecting the cells with a transgene plasmid comprising a rAAVrh74.MHCK7.microdystrophin construct, a plasmid comprising an AAV rep gene and an AAV cap gene, and an adenovirus helper plasmid.

In some aspects, the transgene plasmid comprising a rAAVrh74.MHCK7.microdystrophin construct comprises: the nucleic acid sequence of SEQ ID NO: 9, nucleotides 55-5021 of SEQ ID NO: 3, or nucleotides 1-4977 of SEQ ID NO: 8. In some aspects, the plasmid comprising an AAV rep gene and an AAV cap gene comprises an AAV2 rep gene and an rAAVrh74 cap gene.

In some aspects, the adenovirus helper plasmid comprises an adenovirus 5 E2A, E4ORF6, and a VA RNA gene.

In some aspects, the suspension seed process further comprises: (g) lysing the cells. In some aspects, the cells are lysed by freeze-thaw, solid shear, hypertonic and/or hypotonic lysis, liquid shear, sonication, high-pressure extrusion, detergent lysis or combinations thereof.

In some aspects, the suspension seed process further comprises: (h) purifying the rAAV by at least one column chromatography step. In some aspects, the at least one column chromatography step comprises an anion exchange chromatography or a size exclusion chromatography or a combination thereof.

### Example 7

### An Open-Label, Systemic Gene Delivery Study Using Commercially Representative Material to Evaluate the Safety of and Expression from rAAVrh74.MHCK7.Microdystrophin Construct in Subjects with Duchenne Muscular Dystrophy

This is a phase 1b open-label study using commercially representative material of rAAVrh74.MHCK7.microdystrophin construct, conducted in boys with Duchenne muscular dystrophy. Initially, twenty patients were enrolled and this Example 7 presents data from the first 11 patients, ages to <8 (e.g., 2 patients ages 4-5; 9 patients ages 6-7) (Cohort 1; up to 20 male DMD ambulatory subjects who are ≥4 to <8 years of age.) (Table 7). The study was later expanded to include Cohort 2 (approximately 6 male DMD ambulatory subjects who are ≥8 to <18 years of age) and Cohort 3 (approximately 6 male DMD non-ambulatory subjects), as further described in Example 8.

TABLE 8
Baseline Demographics: First 11 Patients of Cohort 1

| | 4-5 age group | | 6-7 age group | | Total | |
|---|---|---|---|---|---|---|
| Characteristic | (N = 2) | | (N = 9) | | (N = 11) | |
| Age, years; Mean | 5.5 | (0.6) | 6.8 | (0.7) | 6.6 | (0.9) |
| (SD) | | | | | | |
| Sex, Male; n (%) | 2 | (100.0) | 9 | (100.0) | 11 | (100.0) |
| Race, White; n (%) | 1 | (50.0) | 6 | (66.7) | 7 | (63.6.) |
| Years since | 2.3 | (0.7) | 2.8 | (1.8) | 2.7 | (1.7) |
| diagnosis of DMD; | | | | | | |
| Mean (SD) | | | | | | |
| Dosing weight (kg); | 24.5 | (0.3) | 23.4 | (4.4) | 23.6 | (4.0) |
| Mean (SD) | | | | | | |
| Body Mass Index | 20.2 | (2.3) | 17.5 | (2.9) | 18.0 | (2.9) |
| (kg/m2); Mean (SD) | | | | | | |
| NSAA at baseline; | 21.5 | (4.9) | 22.6 | (3.2) | 22.4 | (3.3) |
| Mean (SD) | | | | | | |

The primary objectives were to evaluate micro-dystrophin expression from rAAVrh74.MHCK7.microdystrophin construct (e.g., commercially representative material) at 12 weeks (Part 1) post-infusion as measured by Western blot of biopsied muscle tissue with corresponding endpoint of change in quantity of micro-dystrophin protein expression from Baseline to Week 12 (Part 1) as measured by Western blot. The secondary objectives were to assess: (1) Micro-dystrophin protein expression by immunofluorescence (IF) fiber intensity at Week 12; (2) Micro-dystrophin expression by IF Percentage of Dystrophin-Positive Fibers (PDPF) at Week 12; and (3) safety.

### Inclusion Criteria for the Study, and Applicable to Examples 7 and 8, are as Follows:

A subject must meet all of the following criteria to be eligible to participate in this study:
1. Cohort 1 only (ambulatory <8 years): Is male at birth, ambulatory, and ≥4 to <8 years of age at the time of Screening and has an NSAA score >17 and ≤26 at the Screening visit.
2. Cohort 2 only (ambulatory ≥8 years): Is male at birth, ambulatory, and ≥8 to <18 years of age at the time of Screening and has an NSAA score ≥15 and ≤26 at the Screening visit.
3. Cohort 3 only (non-ambulatory): Is male at birth and has been non-ambulatory for a minimum of 9 months, with a NSAA walk score of “0” and inability to perform the 10 MWR at Screening visit, and with a PUL entry item score ≥2. Onset of loss of ambulation is defined as participant- or caregiver-reported age at continuous wheelchair use, approximated to the nearest month.
4. Has a definitive diagnosis of DMD prior to Screening based on documentation of clinical findings and prior confirmatory genetic testing using a clinical diagnostic genetic test.
5. Has an indication of symptomatic muscular dystrophy:
   CK elevation >1000 U/L and
   Cohorts 1 and 2 only (ambulatory): Below 95 percent predicted time on 100MWR.
6. Able to cooperate with motor assessment testing.
7. Stable weekly dose equivalent of oral corticosteroids for at least 12 weeks before Screening and the dose is expected to remain constant (except for modifications to accommodate changes in weight) throughout the first year of the study.
8. Has rAAVrh74 antibody titers <1:400 (ie, not elevated) as determined by an ELISA.
9. Subjects who are sexually active must agree to use, for the entire duration of the study, a condom and the female sexual partner must also use a medically acceptable form of birth control (eg, oral contraceptive).
10. Has (a) parent(s) or legal guardian(s) or is a subject ≥18 years of age who is (are) able to understand and comply with the study visit schedule and all other protocol requirements.
11. Is willing to provide informed assent or consent (if applicable) and has (a) parent(s) or legal guardian(s) or is a subject ≥18 years of age who is (are) willing to provide written informed consent for the subject to participate in the study.
   Exclusion Criteria for the Study were as Follows:

A subject who meets any of the following criteria will be excluded from this study:
1. Has left ventricular ejection fraction <40% on the Screening ECHO or clinical signs and/or symptoms of cardiomyopathy.
2. Cohorts 2 and 3 only (ambulatory ≥8 years and non-ambulatory): FVC <50% of predicted value at Screening and/or requirement for nocturnal ventilatory support.
3. Major surgery within 3 months prior to Day 1 or planned surgery for any time during this study.
4. Presence of any other significant genetic disease other than DMD.
5. Has serological evidence of current, chronic, or active human immunodeficiency virus, hepatitis C, or hepatitis B infection.
6. Has a diagnosis of an autoimmune disease.
7. Has a concomitant illness or requirement for chronic drug treatment that in the opinion of the Investigator creates unnecessary risks for gene transfer.
8. Has a medical condition or extenuating circumstance that, in the opinion of the Investigator, might compromise the subject's ability to comply with the protocol required testing or procedures or compromise the subject's wellbeing, safety, or clinical interpretability.
9. Has a symptomatic infection (eg, upper respiratory tract infection, pneumonia, pyelonephritis, meningitis) within 4 weeks prior to Day 1.
10. Demonstrates cognitive delay or impairment that could confound motor development in the opinion of the Investigator.
11. Treatment with any of the following therapies according to the time frames specified:
   Any time:
      Gene therapy
      Cell based therapy (eg, stem cell transplantation)
      CRISPR/Cas9, or any other form of gene editing
   Within 12 weeks of Day 1:
      Use of human growth factor, or vamorolone
   Within 6 months of Day 1:
      Any investigational medication
      Cohort 1 only: Any treatment designed to increase dystrophin expression (eg, Translarna™, EXONDYS 51, VYONDYS 53, VILTEPSO™). NOTE: Subjects in Cohort 2 and 3 on these treatments are expected to stop prior to Day 1. Treatments designed to increase dystrophin expression may be resumed and/or started after Week 72
12. Has received a live virus vaccine within 4 weeks or inactive vaccine within 2 weeks of the Day 1 visit or expects to receive a vaccination during the first 3 months after Day 1.
13. Has abnormal laboratory values considered clinically significant including but not limited to:
   Gamma-glutamyl transferase (GGT) >2× upper limit of normal (ULN)
   Total bilirubin >ULN. Note that elevations in total bilirubin deemed due to Gilbert's syndrome are not exclusionary.
   White blood cell count >18,500 per μl
   Platelets <150,000 per μL
14. Subject or family does not want to disclose subject's study participation with general practitioner/primary care physician and other medical providers.

In the opinion of the Investigator, the subject is not likely to be compliant with the study protocol. All subjects received rAAVrh74.MHCK7.microdystrophin construct (1.33×10¹⁴ vg/kg) (e.g., commercially representative material) by single IV infusion.

### Efficacy Assessments:

A muscle biopsy for evaluation of micro-dystrophin expression was collected from all subjects at baseline and week 12. Muscle biopsies were collected using open biopsy or a VACORA core biopsy. The biopsy required collection of muscle tissue from the medial gastrocnemius muscle. If the medial gastrocnemius muscle was not viable, prior approval from the Sponsor was required for using an alternate muscle.

The biopsy sample was used to quantify transgene expression by Western blot, IF intensity, and PDPF.

A mean vector genome copies per nucleus, and change from baseline, was determined to be 3.87(±2.4). A mean percent normal micro-dystrophin expression, and change from baseline, was determined to be 55.4% (±43.4), as determined by Western blot. As determined by immunofluorescence, a mean percentage of dystrophin-positive fibers was determined to be 70.5% (57.7% (±22.2) change from baseline) with a mean intensity of 116.9% (75.9%±46.4 change from baseline). These results are consistent with the placebo group patients of Example 3, which were administered the material described in Example 4 (e.g., mean vector copies per nucleus—2.62; % of normal expression—51.7%; % of dystrophin positive fibers—79.2%; % intensity 100.6%).

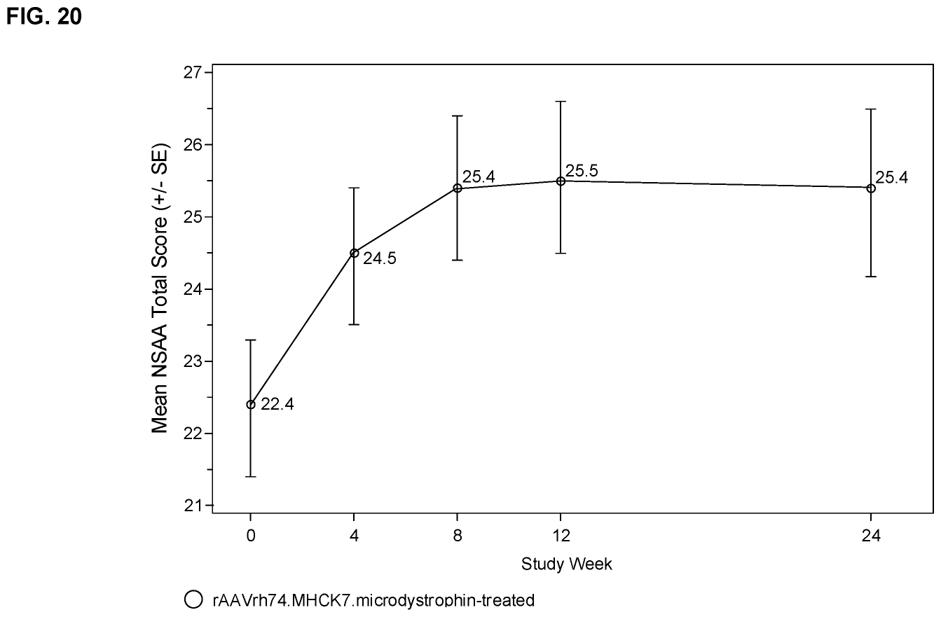

FIG. 20 shows graphically the mean NSAA Score from Cohort 1 (first 11 patients treated with rAAVrh74.MHCK7.microdystrophin). The first 11 patients improved 3 points from baseline. 6 to 7 year olds (n=9) improved 2.9 points from baseline. Each time point represents the 11 patients.

Safety of the commercially representative material was consistent with prior experience with rAAVrh74.MHCK7.microdystrophin construct. 79 treatment-emergent adverse events were observed in 11 patients. The most common adverse event was vomiting, with a typical onset within the first week, mild, and was treated with standard antiemetics. Increase in liver enzymes were transient and responsive to steroids with no signs of impaired liver function in any patient. Serious adverse events in two patients were observed and fully resolved. One patient had increased transaminases and was treated with intravenous steroids. One patient had nausea and vomiting. There were no adverse events suggestive of a complement-mediated event.

Overall, the characteristics of rAAVrh74.MHCK7.microdystrophin construct were confirmed with the commercially representative material. Robust transduction (e.g., 3.87 mean vector genome copies per nucleus) was observed. Mean robust expression with proper localization to the sarcolemma membrane was observed (e.g., Western blot—55.4%; Positive Fibers—70.5%; Intensity—116.9%). A safe, well tolerated, and consistent safety profile was observed with administration of the commercially representative material. No clinical complement manifestations were observed. Collectively, these results provide confirmation of the manufacturing process and analytics, and are sufficient to supply the Duchenne population.

### Example 8

The trials and studies described in Example 7 are expanded to approximately 32 subjects across 3 cohorts: Cohort 1 consists of up to 20 male DMD ambulatory subjects who are ≥4 to <8 years of age; Cohort 2 consists of approximately 6 male DMD ambulatory subjects who are ≥8 to <18 years of age; and Cohort 3 consists of approximately 6 male DMD non-ambulatory subjects.

The first 2 enrolled subjects in each cohort will be sentinel subjects, dosed at least 1 week apart. In Cohorts 2 and 3 combined, at least 3 subjects who weigh <50 kg will be enrolled, and at least 3 subjects who weigh ≥50 kg will be enrolled. The study will consist of 4 periods as follows:
1. An up to approximately 3-week Screening Period (pre-infusion) during which disease characteristics and Baseline therapy will be assessed, and the pre-infusion evaluation is completed.
2. A Baseline Period (pre-infusion), which begins when eligibility is confirmed and ends on the day prior to the Day 1 infusion during which baseline assessments will be completed.
3. An Infusion Period during which a single intravenous (IV) infusion of open-label rAAVrh74.MHCK7.micro-dystrophin will be administered to all subjects within 31 days of obtaining the rAAVrh74 enzyme-linked immunosorbent assay (ELISA) sample. Subjects who weigh <70 kg on Day 1 will receive IV rAAVrh74.MHCK7.micro-dystrophin (1.33×10¹⁴ vg/kg); subjects who weigh ≥70 kg on Day 1 will receive a total fixed dose of rAAVrh74.MHCK7.micro-dystrophin of 9.31×10¹⁵ vg, which is equivalent to the dose of 1.33×10¹⁴ vg/kg for a 70 kg subject. Starting the day prior to the infusion, subjects will receive at least 1 mg/kg of a glucocorticoid (prednisone equivalent) daily in addition to their Baseline stable oral corticosteroid dose for at least 60 days after the infusion; the 1 mg/kg/day added steroid dosing will be followed up to a total daily dose of 60 mg/day (except for added steroids in the event of relevant GGT increases and/or other clinically significant liver function abnormalities). Post-infusion added glucocorticoid for immunosuppression should be increased to 2 mg/kg daily (or if the subject is on 60 mg/day fixed dose, this should be increased to 120 mg/day) if GGT level is confirmed to be ≥150 U/L or there are other clinically significant liver function abnormalities following infusion. The Investigator may make subsequent adjustments to immunosuppressive therapy in reaction to the subsequent course of acute liver injury or other AEs. A hepatologist must be consulted for serious or severe elevations in hepatic biochemistries (including GGT, bilirubin, and ALT relative to Baseline), or for elevations that do not respond to 2 mg/kg/day or 120 mg/day, respectively. In this situation, IV bolus steroids may be considered. The dosages indicated in this Example 8, and in Example 7, are determined using a linearized DNA qPCR standard. Otherwise, the dosages indicated herein are determined utilizing a supercoiled DNA qPCR standard. For example, the dosage of 1.33×10¹⁴ vg/kg indicated in Examples 7 and 8, corresponds to the dosage of 2×10¹⁴ vg/kg otherwise described herein. The trials and studies described in Examples 7 and 8 above were and are carried out utilizing the rAAVrh74.MHCK7.micro-dystrophin construct set forth in SEQ ID NO: 9; as set forth in SEQ ID NO: 8, nucleotides 1-4977; or as set forth in SEQ ID NO: 6; nucleotides 56-5022.
4. A 260-week Follow-Up Period (post-infusion) during which safety, efficacy, and expression parameters will be evaluated. Subjects will be expected to attend both remote and in-person visits to complete required procedures/assessments. Part 1 of the Follow-Up Period will begin post infusion (Day 1) through Week 12. Part 2 of the Follow-Up Period will begin post Week 12 through Week 260. The first 12 weeks (Part 1) following infusion require frequent visits (nearly weekly). Additional unscheduled visits are allowed per the Investigator's clinical judgement. For subjects who complete the study, the last study visit will occur at Week 260. For subjects who prematurely discontinue post-infusion follow-up, an Early Termination visit will be required; however, each subject should be strongly encouraged to continue study follow-up until 260 weeks post infusion.

The study is further expanded to include Cohort 4, which consists of approximately 6 male DMD ambulatory subjects who are ≥3 to <4 years of age. Subjects in Cohort 4, who are not on oral corticosteroids for their DMD at screening, will start prednisone/prednisolone at 1.5 mg/kg/day 1 week prior to the infusion, which will continue for at least 60 days after the infusion.

The following exclusion criteria is specified for Cohorts 1 and 4 only: Any treatment designed to increase dystrophin expression (e.g., Translarna™, EXONDYS 51, VYONDYS 53, VILTEPSO™). Treatments designed to increase dystrophin expression may be resumed and/or started after Week 72.

### Example 9

### Genetic Diagnostics

In additional studies, or alternatively, in the studies described above, as applicable, subjects must have a definitive diagnosis of DMID prior to screening based on documentation of clinical findings and prior confirmatory genetic testing using a clinical diagnostic genetic test.

The genetic test is used to genotype a patient for at least one mutation in the human dystrophin (DMD) gene. As used herein, “genotyping” refers to a process of determining the specific allelic composition of a cell and/or subject at one or more positions within the genome, e.g. by determining the nucleic acid sequence at that position. Genotyping refers to a nucleic acid analysis and/or analysis at the nucleic acid level.

In one aspect, the method of treating DMD described herein further comprises genotyping the human dystrophin (DMD) gene of the human subject prior to administering the composition to said human subject.

In some aspects, the human dystrophin gene (DMD) of a subject is genotyped to characterize a mutation in the gene that would be amenable to treatment with the compositions disclosed herein.

In some aspects, the subject is genotyped for at least one mutation in exons 18 to 79 of the DMD gene. Specifically, the subject is genotyped for a mutation that is expected to lead to an absence of the dystrophin protein in the patient. For example, in some aspects, the subject is genotyped for a frameshift deletion, frameshift duplication, premature stop, or other pathogenic variant in the DMD gene fully contained between exons 18 to 79. Identification of at least one of these mutations indicates that a patient is eligible for treatment according to the present disclosure.

In some aspects, patient genotyping of the DMD gene can provide results indicating that the subject is not eligible for a treatment according to the present disclosure. For example, a genotyping result that demonstrates a mutation between or including exons 1-17, an in-frame deletion, in-frame duplication, variant of uncertain significance (“VUS”), a mutation that is fully contained within exon 45 indicates that the patient is not eligible for a treatment according to the present disclosure.

Numerous genotyping techniques are known to those skilled in the art.

### Example 10

### A Phase 3 Multinational, Randomized, Double-Blind, Placebo Controlled Systemic Gene Delivery Study to Evaluate the Safety and Efficacy of Delandistrogene Moxeparvovec in Subjects with Duchenne Muscular Dystrophy

This is a randomized, double-blind, placebo-controlled 2-part study of systemic gene delivery of the study drug rAAVrh74.MHCK7.microdystrophin (as also described in Examples 6-8 above) in approximately 120 male DMD ambulatory subjects ≥4 to <8 years of age. Randomization will be stratified by age group at the time of randomization (≥4 to <6 years vs. ≥6 to <8 years) and NSAA total score at Screening (≤22 vs. >22); at least 50% of subjects must be randomized to the ≥4 to <6 years age group at the time of randomization. All patients will have the opportunity to receive intravenous (IV) study drug (1.33×10¹⁴ vg/kg) in either Part 1 or Part 2. In the treatment arm, participants will receive single intravenous (IV) infusion of the study drug on Day 1. Then, participants will receive a single IV infusion of matching placebo at year 2. In the placebo arm, the participants will receive matching placebo IV infusion on Day 1. Then, participants will have the opportunity to receive a single IV infusion of the study drug at Year 2. The study will consist of 4 periods as follows:
A Screening Period (pre-infusion) which begins a maximum of 31 days prior to the Day 1 infusion and during which disease characteristics and baseline therapy will be assessed, and the pre-infusion evaluation will be completed.
A Baseline Period (pre-infusion) which begins when eligibility is confirmed and ends on the day prior to the Day 1 infusion during which baseline assessments will be completed.
An Infusion Period during which a single intravenous (IV) infusion of blinded STUDY DRUG or placebo will be administered within 31 days of obtaining the rAAVrh74 enzyme-linked immunosorbent assay (ELISA) sample. Approximately 60 subjects will receive IV study drug (1.33×10¹⁴ vg/kg; as determined using a linear DNA qPCR standard) and approximately 60 subjects will receive placebo (saline, 0.9% sodium chloride solution) in the Infusion Period in Part 1. In the Infusion Period in Part 2, subjects who received placebo in Part 1 will receive IV study drug, and subjects who received study drug in Part 1 will receive placebo. All subjects, parents/caregivers, investigators, and site staff, with the exception of the unblinded site pharmacist, will be blinded to subject treatment (study drug or placebo). Starting the day prior to the infusion, all subjects will receive additional glucocorticoid (prednisone equivalent) for at least 60 days.
A 104-week Follow-up Period (post Part 1 infusion) during which safety and efficacy parameters will be evaluated in Part 1 and Part 2. Subjects will be expected to attend both remote and in-person visits to complete required procedures/assessments. Additional, unscheduled visits are allowed per the Investigator's clinical judgement. For subjects who complete the study, the last study visit will occur at Part 2 Week 52. For subjects who prematurely discontinue post-infusion follow-up, an end of study/early termination visit will be required; however, each subject should be strongly encouraged to continue study follow-up until 52 weeks post each infusion.

### Inclusion/Exclusion Criteria:

### Inclusion Criteria:

A subject must meet all of the following criteria to be eligible to participate in this study:
1. Is male at birth, ambulatory, and ≥4 to <8 years of age at the time of randomization.
2. Has a definitive diagnosis of DMD prior to Screening based on documentation of clinical findings and prior confirmatory genetic testing using a clinical diagnostic genetic test. Genetic report must describe a frameshift deletion, frameshift duplication, premature stop (“nonsense”), canonical splice site mutation, or other pathogenic variant in the DMD gene fully contained between exons 18 to 79 (inclusive) that is expected to lead to absence of dystrophin protein.
   a. Mutations between or including exons 1-17 are not eligible.
   b. In-frame deletions, in-frame duplications, and variants of uncertain significance (“VUS”) are not eligible.
   c. Mutations fully contained within exon 45 (inclusive) are not eligible.
3. Able to cooperate with motor assessment testing.
4. Has a NSAA score >16 and <29 at the Screening visit.
5. Has a time to rise from floor <5 seconds at the Screening visit.
6. Stable daily dose of oral corticosteroids for at least 12 weeks before Screening and the dose and regimen are expected to remain constant (except for modifications to accommodate changes in weight) throughout the study.
7. Has rAAVrh74 antibody titers <1:400 (i.e., not elevated) as determined by an ELISA.
8. Subjects who are sexually active must agree to use, for the entire duration of the study, a condom and the female sexual partner must also use a medically acceptable form of birth control (e.g., oral contraceptive).
9. Has (a) parent(s) or legal guardian(s) who is (are) able to understand and comply with the study visit schedule and all other protocol requirements.
10. Is willing to provide informed consent (if applicable) and has (a) parent(s) or legal guardian(s) who is (are) willing to provide informed consent for the subject to participate in the study.

### Exclusion Criteria

A subject who meets any of the following criteria will be excluded from this study:
1. Has left ventricular ejection fraction <40% on the screening ECHO or clinical signs and/or symptoms of cardiomyopathy.
2. Major surgery within 3 months prior to Day 1 or planned surgery or procedures that would interfere with the conduct of the study for any time during this study.
3. Presence of any other clinically significant illness, including cardiac, pulmonary, hepatic, renal, hematologic, immunologic, or behavioral disease, or infection or malignancy or concomitant illness or requirement for chronic drug treatment that in the opinion of the Investigator creates unnecessary risks for gene transfer or a medical condition or extenuating circumstance that, in the opinion of the Investigator, might compromise the subject's ability to comply with the protocol required testing or procedures or compromise the subject's wellbeing, safety, or clinical interpretability.
4. Has serological evidence of current, chronic, or active human immunodeficiency virus, hepatitis C, or hepatitis B infection.
5. Has a symptomatic infection (eg, upper respiratory tract infection, pneumonia, pyelonephritis, meningitis) within 4 weeks prior to Day 1.
6. Demonstrates cognitive delay or impairment that could confound motor development in the opinion of the Investigator.
7. Treatment with any of the following therapies according to the time frames specified:
   Any time:
      Gene therapy
      Cell based therapy (e.g., stem cell transplantation)
      CRISPR/Cas9, or any other form of gene editing
   Within 12 weeks of Day 1 and any time during the study:
      Use of human growth factor or vamorolone
   Within 6 months of Day 1 and any time during the study:
      Any investigational medication
      Any treatment designed to increase dystrophin expression (e.g., Translarna™, EXONDYS 51™, VILTEPSO™)
8. Has received a live virus vaccine within 4 weeks or inactive vaccine within 2 weeks of the Day 1 visit or expects to receive a vaccination during the first 3 months after Day 1.
9. Has abnormal laboratory values considered clinically significant including but not limited to:
Gamma-glutamyl transferase >2× upper limit of normal (ULN)
Glutamate dehydrogenase (GLDH) >15 U/L
Total bilirubin >ULN. Note; elevations in total bilirubin confirmed to be due to Gilbert's syndrome are not exclusionary.
White blood cell count >18,500 per μl
Platelets ≤150,000 per μL
10. Family does not want to disclose subject's study participation with general practitioner/primary care physician and other medical providers.
11. In the opinion of the Investigator, the subject is not likely to be compliant with the study protocol.

### Genetic Diagnostics

Subjects must have a definitive diagnosis of DMD prior to Screening based on documentation of clinical findings and prior confirmatory genetic testing using a clinical diagnostic genetic test. Genetic report must describe a frameshift deletion, frameshift duplication, premature stop, or other pathogenic variant in the DMD gene fully contained between exons 18 to 79 (inclusive) that is expected to lead to absence of dystrophin protein.
a. Mutations between or including exons 1-17 are not eligible.
b. In-frame deletions, in-frame duplications, and variants of uncertain significance (“VUS”) are not eligible.
c. Mutations that are fully contained within exon 45 (inclusive) are not eligible.

### Statistical Methods:

### Sample Size:

The sample size of this study is based on the power for the primary efficacy endpoint, change in NSAA total score from Baseline to Week 52 (Part 1).

Assuming a standard deviation of 3.5 in all subjects and a 10% dropout rate at Week 52 (Part 1), with a Type 1 error of 0.05 (2-sided), a sample size of 120 with 1:1 randomization ratio will provide approximately 90% power to detect a mean difference of 2.2 in change in NSAA total score from Baseline to Week 52 (Part 1) between the study drug group and placebo group.

The study is also powered for the age group of ≥4 to <6 years. The study will enroll at least 60 subjects with age of ≥4 to <6 years. Assuming a standard deviation of 3.2 for the primary endpoint in the age group of ≥4 to <6 years and a 10% dropout rate at Week 52 (Part 1), with a Type 1 error of 0.05 (2-sided), a sample size of 60 with 1:1 randomization ratio will provide at least 80% power to detect a mean difference of 2.5 in change in NSAA total score from Baseline to Week 52 (Part 1) between the study drug group and placebo group.

A testing procedure to adjust for multiplicity will be used to control the overall Type 1 error at a 2-sided level of 0.05 with the details to be specified in the SAP.

### Randomization

Subjects will be randomized in a 1:1 ratio to receive either STUDY DRUG or placebo by single IV infusion. Subjects who received STUDY DRUG in Part 1 of the study will receive placebo in Part 2. Subjects who received placebo in Part 1 of the study will have the opportunity to receive study drug in Part 2.

Randomization will be stratified by age group at the time of randomization (≥4 to <6 years or ≥6 to <8 years) and NSAA total score (≤22 or >22) at Screening; at least 50% of subjects must be randomized to the ≥4 to <6 years age group at the time of randomization. All patients will have the opportunity to receive intravenous (IV) study drug (1.33×10¹⁴ vg/kg) in either Part 1 or Part 2.

### Corticosteroids

Subjects must be on a stable daily dose of oral corticosteroids for at least 12 weeks before the initial Screening visit, with the dose remaining constant (except for modifications to accommodate changes in weight) throughout the study. All changes to corticosteroid type, dosing frequency, the dates of start and end of corticosteroid dosage, and dosage will be recorded in the subject's source documents and on the eCRF.

### Pre-Infusion Immunosuppressants

The day before the infusion (STUDY DRUG or placebo), subjects will begin additional glucocorticoid (prednisone equivalent) for immunosuppression, in addition to their baseline stable oral corticosteroids for DMD. Subjects on baseline daily corticosteroid dosing for their DMD will take their usual DMD corticosteroid dose IN ADDITION to the added 1 mg/kg/day immunosuppressive dose. The 1 mg/kg/day dosing will be followed up to a total daily dose of 60 mg/day.

### Post-Infusion Immunosuppressants

For the first 60 days following the infusion, subjects will remain on their baseline daily stable oral corticosteroid dose for DMD, and IN ADDITION, will take 1 mg/kg/day of a glucocorticoid (prednisone equivalent) for immunosuppression. Earlier tapering to manage an adverse event (AE) may be permitted with Medical Monitor approval. The 1 mg/kg/day dosing will be followed up to a total daily dose of 60 mg/day, except for added steroids in the event of GGT increases and/or other clinically significant liver function abnormalities.

If a subject cannot tolerate oral immunosuppressive glucocorticoids due to vomiting, then the glucocorticoids should be administered intravenously.

Post-infusion added glucocorticoid for immunosuppression should be increased if GGT level is confirmed to be ≥150 U/L or there are other clinically significant liver function abnormalities following infusion. The Investigator may make subsequent adjustments to immunosuppressive therapy in reaction to the subsequent course of acute liver injury or other AEs. A hepatologist must be consulted for serious or severe elevations in hepatic biochemistries (including GGT, bilirubin, GLDH, and ALT relative to baseline), or for elevations that do not respond to 2 mg/kg/day or 120 mg/day, respectively. In this situation, IV bolus steroids may be considered.
If the subject is on 1 mg/kg of added steroid for immunosuppression in addition to their DMD steroid, this dose should be increased to 2 mg/kg of added steroid for immunosuppression to be taken in addition to their DMD steroid.
If the subject is on 60 mg/day fixed dose, this should be increased to 120 mg/day.

Subjects with normal GGT values and no signs of acute liver injury at Day 60 should be tapered off their immunosuppressive glucocorticoid over 2 weeks. The duration of tapering may be adjusted to manage AEs, per Investigator discretion, but continuation of steroids at a dose exceeding the baseline daily regimen beyond day 90 should be discussed with the Medical Monitor. Immunosuppressive glucocorticoids in subjects with elevated GGT values and/or signs of acute liver injury at Day 60 should be managed as above until GGT values normalize (or are clearly trending toward normal) and all signs of acute liver injury resolve, at which point they should be tapered off their immunosuppressive glucocorticoid over 2 weeks. Once the additional steroids for immunosuppression have been tapered off, they should remain on their baseline daily steroid regimen for DMD, with any required adjustment for weight.

### Efficacy Analyses:

### North Star Ambulatory Assessment (NSSA)

The primary endpoint and some secondary endpoints will be tested in a hierarchical manner using an appropriate multiple-testing approach that provides strong control of the familywise Type 1 error rate at a 2-sided 0.05 level.

For the primary endpoint of change in NSAA total score from Baseline to Week 52 (Part 1), summary statistics will be provided by treatment group for NSAA total score at Baseline, each post-baseline visit in Part 1, and for change from Baseline to each post-baseline visit in Part 1. For the Baseline and Week 52 (Part 1) visits where 2 scores of NSAA will be collected, the mean NSAA score will be used in the analysis.

As the primary analysis, a restricted maximum likelihood-based mixed model repeated measures analysis will be used to compare the 2 treatment groups for change in NSAA total score from Baseline to Week 52 (Part 1). In this model, the response vector consists of the change from Baseline in NSAA total score at each post-baseline visit in Part 1. The model will include the covariates of treatment group (categorical), visit (categorical), treatment group by visit interaction, age group at Randomization.

The NSAA is a clinician-administered scale that rates performance on various functional activities (Mazzone, E et. al., Neuromuscul Disord. 20(11):712-716 (2010)). It was designed to be used in boys with DMD who are able to stand, and it has been used in DMD boys of this study's age range (≥4 and <8 years) (Connolly A M et al., “Motor and cognitive assessment of infants and young boys with Duchenne Muscular Dystrophy: results from the Muscular Dystrophy Association DMD Clinical Research Network,” Neuromuscul Disord. 23(7):529-539 (2013)); Mercuri E et al., “Revised North Star Ambulatory Assessment for Young Boys with Duchenne Muscular Dystrophy,” PLoS One 11(8):e0160195 (2016)).

During this assessment, subjects perform 17 different functional activities, including the 10MWR, rising from a sit to a stand, standing on 1 leg, climbing a box step, descending a box step, rising from lying to sitting, rising from the floor, lifting head off floor, standing on heels, and jumping.

Subjects will be graded as follows: 2=normal, no obvious modification of activity; 1=modified method but achieves goal independent of physical assistance from another; and 0=unable to achieve goal independently.

Details on administration of the NSAA are provided in the Clinical Evaluator Manual.

Two NSAA scores will be collected on 2 separate days at Baseline, 2 separate days at Week 52 in Part 1, and 2 separate days at Week 52 in Part 2.

Time to Rise from the Floor

The time to rise from the floor test is part of the NSAA (item 11) and quantifies the time required for the subject to stand in an upright position with arms by sides, starting from the supine position with arms by sides (Henricson, E K et al., Muscle Nerve 48(1): 55-67 (2013)). The time required for the subject to complete the task will be recorded during the NSAA administration. As with NSAA, the time to rise from the floor will be collected on 2 days at the Baseline and Week 52 visits in Part 1 and Part 2.

### 10-Meter Walk/Run

The timed 10 MWR is part of the NSAA (item 17) and quantifies the time required for the subject to run or walk 10 meters (on a straight walkway) from a standing position (McDonald, C M et al., Muscle Nerve 48(3):357-368 (2013)). The subject is encouraged to run past the 10-meter mark. The time required for the subject to cover the distance will be recorded during the NSAA administration. As with NSAA, timed 10MWR will be collected on 2 days at the Baseline and Week 52 visits in Part 1 and Part 2.

### Time to Ascend 4 Steps

The timed 4-step test quantifies the time required for the subject to ascend 4 standard steps (each step 6 inches in height) (Bushby, K et al., Clin Investig (Lond). 1(9):1217-1235 (2011)). The time required for the subject to climb up 4 standard-sized steps will be recorded.

### 100-Meter Walk/Run

The 100MWR quantifies the time required for the subject to run or walk 100 meters (on a straight walkway) from a standing position (Alfano, L N et al., Neuromuscul Disord. 27(5):452-457 (2017)). The subject is encouraged to run past the 100-meter mark. The time required for the subject to cover the distance will be recorded.

### Wearable Device

Subjects will be provided with a wearable device to collect daily physical activities. The purpose of the wearable device is to accurately measure the movement and activity levels of the subject during normal daily living, outside of investigational site visits. The device consists of 2 sensors, one of which is worn on each ankle. As the wearable device battery requires recharging daily after use, the sensors will not be worn at night. Site personnel, the subject, and parents/caregivers will be trained on the correct use of the device.

During the Pre-Infusion Period, the wearable device will be worn daily on both ankles for 3 weeks to capture baseline values. During the Follow-Up Period, the wearable device will be worn daily on both ankles for 3 weeks prior to the Weeks 12, 24, 36, and 52/early termination clinic visits in Part 1 and Part 2. The wearable device will not be worn during clinic visits, but subjects will resume use immediately following completion of in-clinic tests.

### Micro-Dystrophin Expression

A muscle biopsy for evaluation of micro-dystrophin expression will be collected from a subset of subjects at Week 12 in Part 1 and Week 12 in Part 2. Muscle biopsies should be collected using open biopsy or a VACORA core biopsy, with Sponsor approval. The biopsy must require collection of muscle tissue from the medial gastrocnemius muscle. If the medial gastrocnemius muscle is not viable, prior approval from the Sponsor is required before using an alternate muscle.

The biopsy sample will be used to quantify micro-dystrophin protein expression by Western blot adjusted for muscle content, and localization of micro-dystrophin will be assessed using immunohistochemistry as IF intensity, and PDPF. Refer to the Biopsy Surgical and Laboratory Manual for additional details on the handling and processing of biopsy tissues.

### Vector Genome and Quantification

Serum will be collected to assess vector quantification at various time points. On Day 1, the sample will be collected approximately 4 to 6 hours following the end of the infusion.

Vector genome copies will be measured in muscle biopsy samples at Week 12 in Part 1 and Week 12 in Part 2 using polymerase chain reaction.

### Creatine Kinase

Creatine kinase levels following study drug infusion will serve as an exploratory efficacy measure.

### Patient-Reported Outcomes Measurement Information System

A subset of subjects based on regional availability will complete the PROMIS measures; participating countries will be outlined in the Study Operations Manual.

PROMIS is a family of instruments developed and validated to assess health-related quality of life (PROMIS Pediatric and Parent Proxy Profile Instruments. Available at http://www.healthmeasures.net/images/PROMIS/manuals/PROMIS_Pediatric_and_Pr oxy_Profile_Scoring_Manual.pdf., 10 Oct. 2018, Bevans, K B et al., Expert Rev Pharmacoecon Outcomes Res. 10(4):385-396 (2010). PROMIS measures have been developed in alignment with the Food and Drug Administration's methodological standards for the assessment of patient-reported outcomes (Bevans, K B et al., Food and Drug Administration. Guidance for Industry Patient-Reported Outcome Measures: Use in Medical Product Development to Support Labeling Claims; https://www.fda.gov/downloads/drugs/guidances/ucm193282.pdf.; 10 Oct. 2018), and are comprised of person-centered items that evaluate and monitor physical, mental, and social health in adults and children. Specific measures for child outcomes in this study will include the following proxy measures:
PROMIS Parent Proxy Item Bank V 2.0—Mobility
PROMIS Parent Proxy SF V 2.0—Upper Extremity—Short Form 8a
PROMIS Parent Proxy SF V 2.0—Fatigue—Short Form 10a

If a subject turns 8 years old within the study period, the following Pediatric PROMIS measures will also be administered and completed by the subject. The parent/caregiver will continue to complete the proxy measure.
PROMIS Pediatric Item Bank V 2.0—Mobility
PROMIS Pediatric Item Bank V 2.0—Upper Extremity—Short Form 8a
PROMIS Pediatric Item Bank V 2.0—Fatigue—Short Form 10a

### Summary of Key Efficacy Assessments in Part 1:

Primary Outcome Measures: Change From Baseline in NSAA Total Score at Week 52.

### Secondary Outcome Measures:

Quantity of Micro-Dystrophin Protein Expression at Week 12 as Measured by Western Blot, in a Subset of Participants (Week 12).
Change From Baseline in Time to Rise From the Floor, Time to Complete 100 and 10 meter Walk/Run, and the Timed Stair Ascend 4 Steps Test at Week 52 (Baseline, Week 52)
Change From Baseline in Stride Velocity 95th Centile (SV95C) Measured by a Wearable Device (Baseline up to Week 52)
Change from Baseline in Patient-Reported Outcomes Measurement Information (PROMIS) Score per Domain at Week 52 (Baseline, Week 52) PROMIS is a family of instruments developed and validated to assess health-related quality of life. Parents will be asked “Taking into account all aspects of your child's observable symptoms, physical ability, ability to perform daily activities and overall health, how would you rate the change in clinical status for your child since the study start? using the following rating scale 1==very much improved, 2=much improved, 3=minimally improved, 4=no change, 5=minimally worse, 6=much worse, and 7=very much worse.”
Part 1: Number of Skills Gained or Improved at Week 52 as Measured by the NSAA (Baseline up to Week 52).

### Example 11

### Evaluation of Cardiac Function in DMD^{mdx} Rats After Systemic Delivery of Delandistrogene Moxeparvovec

The DMD^{mdx} rat model is a valuable small animal model of Duchenne muscular dystophy (DMD) with phenotypic properties very close to the human DMD pathology. See, Larcher T. et al., Plos One 9(10):el10371 (2014); Szabó, P. L. et al., Disease Models & Mechanisms 14: dmm047704. doi:10.1242/dmm.047704 (2021). The objective of this study was to evaluate the cardiac function of male Sprague-Dawley DMD mutated rats (DMD^{mdx} rats), aged 21-28 days, following systemic delivery of a single dose (1.33×10¹⁴ vg/kg) of delandistrogene moxeparvovec, compared to saline.

Study endpoints included safety pharmacologic measurements, targeted micro-dytrophin expression in skeletal and cardiac muscle, biodistribution, histology, and echocardiogram to evaluate cardiac function. The treatment cohort included DMD^{mdx} rats that were systemically delivered a single dose (1.33×10¹⁴ vg/kg) of the rAAV rh74.MHCK7.micro-dystrophin vector described herein (delandistrogene moxeparvovec). The control cohort included DMD^{mdx} rats that received saline. Cardiac function between the two cohorts was assessed at a 12-week and 24-week time point, as described below.

### Targeted Expression of Micro-Dystrophin in Skeletal and Cardiac Muscle

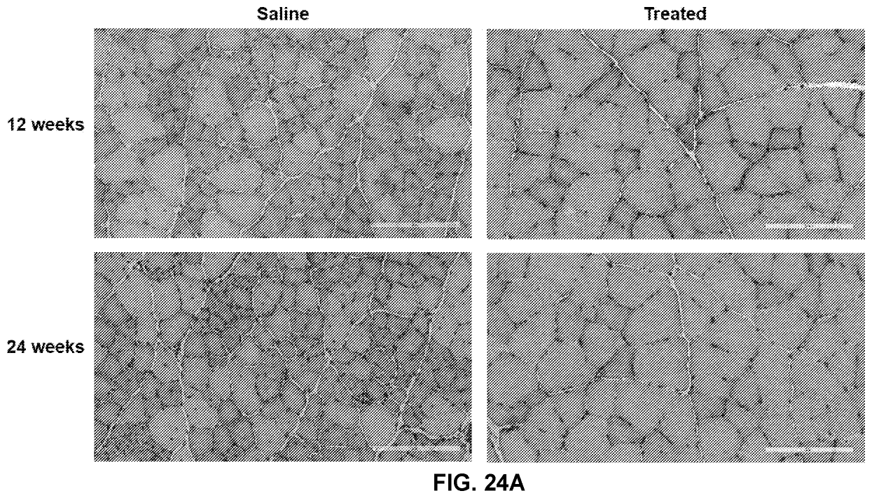

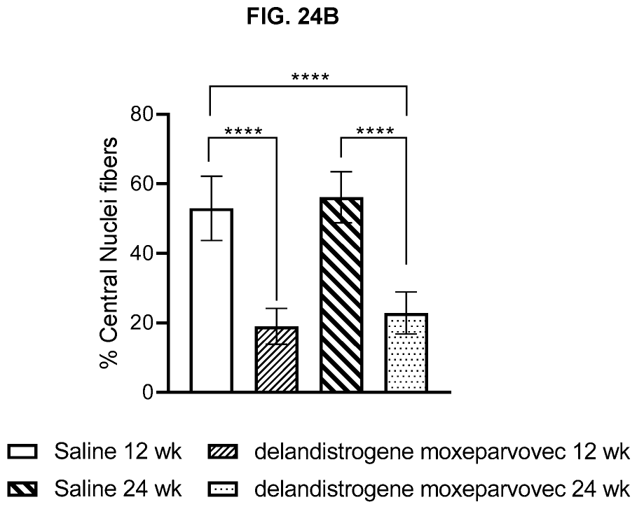

Systemic delivery of delandistrogene moxeparvovec has demonstrated targeted expression in both skeletal and cardiac muscles. The visualization of micro-dystrophin expression in skeletal and cardiac muscle (immunofluorescence) of DMD^{mdx} rats after treatment with delandistrogene moxeparvovec vs. saline is shown in FIGS. 21A-21C. FIGS. 21A-21C demonstrate micro-dystrophin expression in skeletal (“LTA”) and cardiac muscle (“HRT”) of DMD^{mdx} rats at 12 weeks (FIG. 21B) and 24 weeks (FIG. 21C) after treatment with delandistrogene moxeparvovec, compared to saline (FIG. 21A)

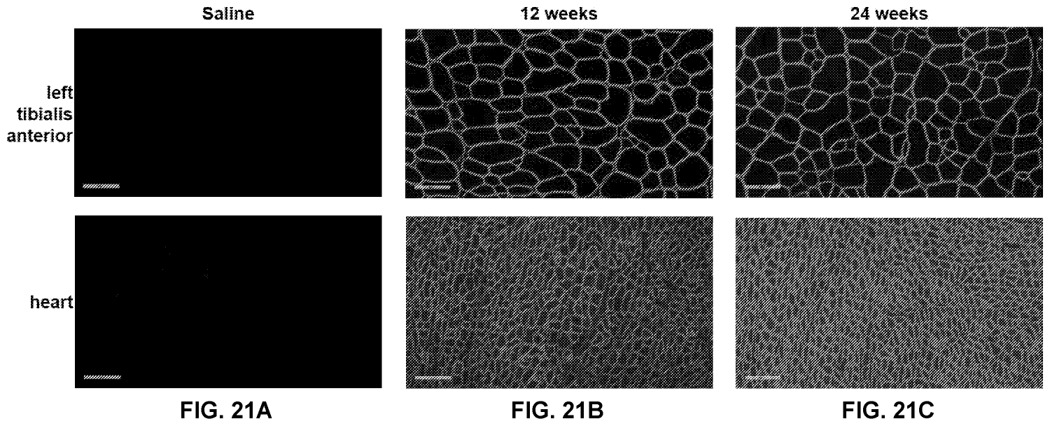

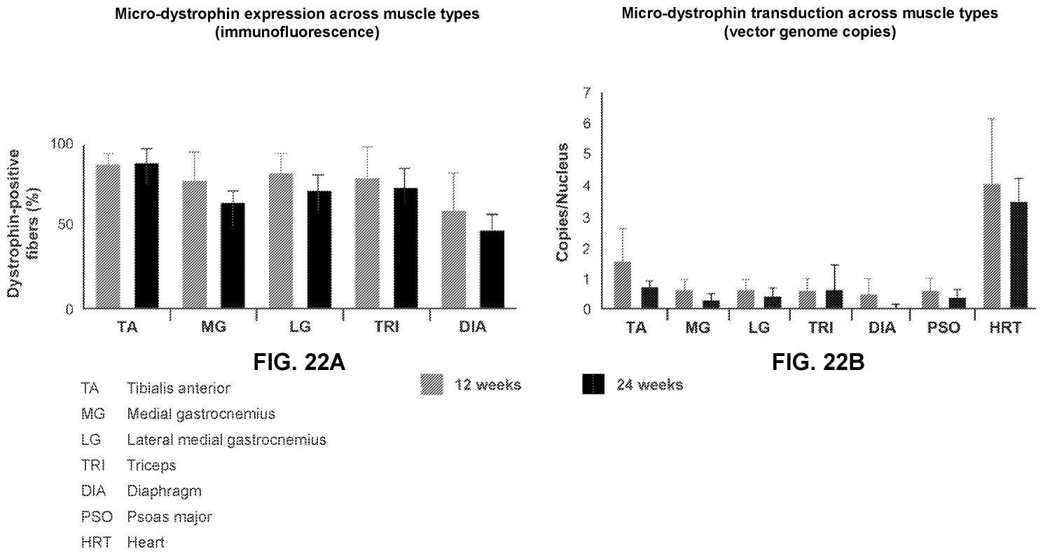

FIGS. 22A-22B depict the quantitation of micro-dystrophin expression (immunofluorescence) (FIG. 22A) and vector transduction (vector genome copies) (FIG. 22B) in several muscle tissues of DMD^{mdx} rats at 12 weeks and 24 weeks after treatment with delandistrogene moxeparvovec. Key: TA=tibialis; HRT=heart; MG=medial gastrocnemius; LG=lateral medial gastrocnemius; DIA=diaphragm; TRI=triceps; PSO=psoas major.

### Reduction in Muscle Degeneration and Fibrosis

A significant reduction in muscle degeneration by central nucleation analysis in skeletal muscle was observed at both 12 weeks and 24 weeks after delandistrogene moxeparvovec administration in DMD^{mdx} rats, compared to saline. FIG. 24A depicts H & E staining of the left medial gastrocnemius muscle, which illustrated improvement in the muscle pathology of delandistrogene moxeparvovec-treated DMD^{MDX} rats as compared to the more severe dystrophic phenotype exhibited in the saline-treated DMD^{MDX} rats.

Quantification of histological parameters in the LMG was also performed. Central nucleation and fiber diameter, markers of muscle degeneration, were examined. See, Pastoret, C. et al., J Neur Sci 129: 97-105 (1995); L. R. Rodino-Klapac et al., Mol Genet. 22:4929-4937 (2013), Potter R. A. et al. Hum Gene Ther. 32:375-89 (2021). Mean central nucleation showed an improvement in the central nucleation (CN) of treated animals, indicating less muscle degeneration in treated animals. Saline-dosed rats exhibited a mean percent positive central nucleation of greater than 50% for both 12 and 24 week cohorts, while delandistrogene moxeparvovec central nucleation was less than 25% for both 12 and 24 week cohorts (FIG. 24B). The differences between saline-treated animals and animals treated with delandistrogene moxeparvovec at 12 and 24 weeks were both statistically significant and demonstrated improvements in the degeneration/regeneration process and a decrease in muscle fiber damage with delandistrogene moxeparvovec treatment. Fiber diameter showed a normalization of the fiber size distribution and increase in the average diameter of fibers with delandistrogene moxeparvovec treatment vs. saline at both 12 and 24 weeks, which is indicative of a normalized muscle environment.

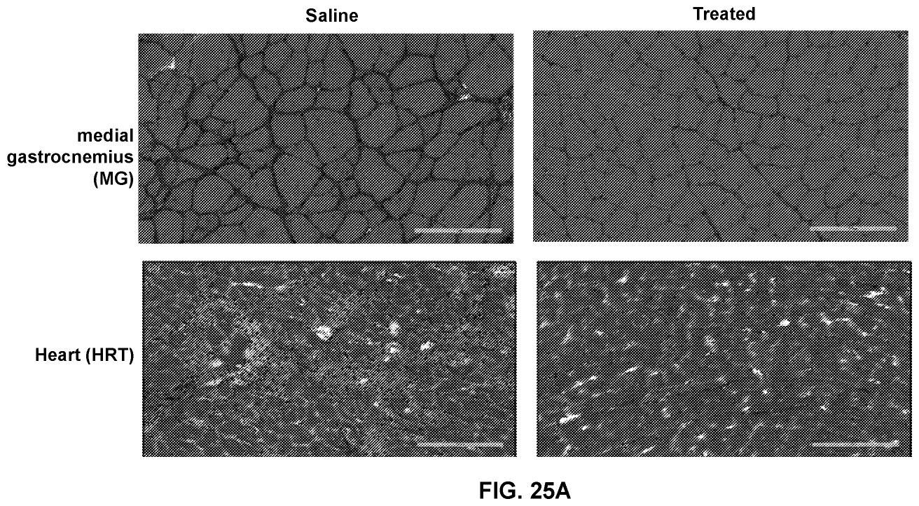

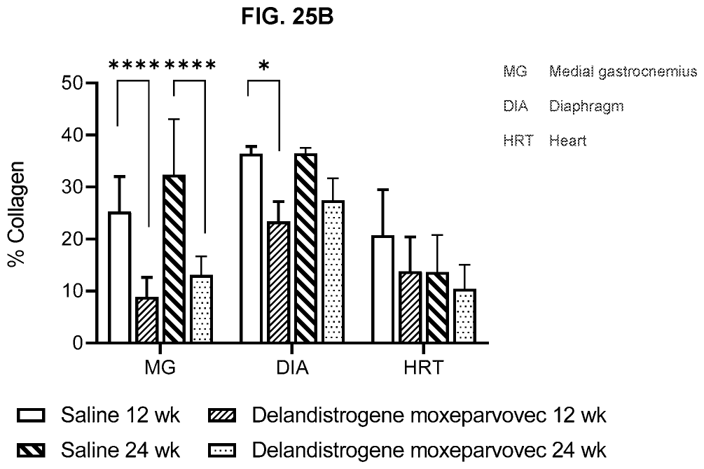

Fibrosis was analyzed by quantifying the percentage of collagen in tissue sections. FIGS. 25A-25B depict an analysis of collagen deposition in skeletal and cardiac muscle. Images in FIG. 25A depict Masson's Trichome staining at 12 weeks post-treatment. The representative images in FIG. 25A show fibrosis of the MG and HRT of delandistrogene moxeparvovec and saline treated animals at 12 weeks post-dosing. Blue staining indicates fibrosis and red stain indicates muscle fibers. Fibrosis was reduced after treatment with delandistrogene moxeparvovec in DMD^{mdx} rats at 12 weeks and 24 weeks, compared to saline. FIG. 25B quantifies collagen deposition in various types of skeletal and cardiac muscle for 12 and 24 weeks post-treatment. Quantification of fibrosis showed that the delandistrogene moxeparvovec-treated cohorts had a lower percentage of fibrotic area per stained muscle section than saline-treated cohorts (FIG. 25B) in all the analyzed tissues. (HRT=heart; MG=medial gastrocnemius; DIA=diaphragm).

### Evaluation of Cardiac Function

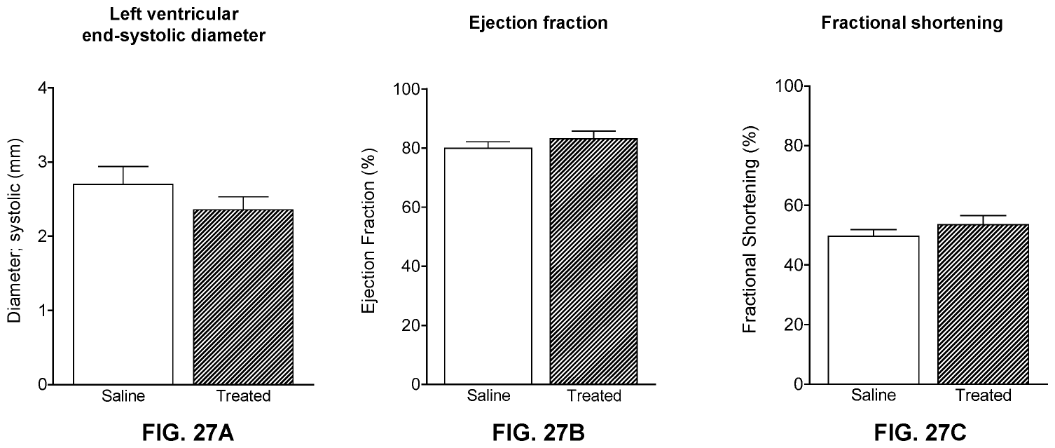

Systolic ventricular performance in the DMD^{mdx} rats was evaluated at 24 weeks by echocardiography, following administration of delandistrogene moxeparvovec, compared to saline. Bar graphs depicting data for the “treated” cohort vs. the control (“saline”) cohort are shown in FIG. 27A (for left ventricular end-systolic diameter (LVESD); FIG. 27B (for ejection fraction (%) (EF))l and FIG. 27C (for fractional shortening (%) (FS)).

Administration of delandistrogene moxeparvovec led to modest improvements in ejection fraction (EF) (FIG. 27B), and fractional shortening (FS) (FIG. 27B) vs. saline-treated DMD^{MDX} rats. A trend toward the reversal of dilatation with delandistrogene moxeparvovec led to mild decreases in LVIDd (left ventricular internal diameter, diastolic) and LVIDs (left ventricular internal diameter, systolic)/LVESD (left ventricular-end systolic diameter), and reduction in end diastolic volume (EDV). The mild decrease in left ventricular end-systolic diameter 24 weeks after treatment with delandistrogene moxeparvovec vs. saline is shown in the bar graphs of FIG. 27A.

The data shown in FIGS. 27A-27C, reflecting three echocardiographic measures of cardiac function, show a positive trend toward sustained cardiac function 24 weeks post treatment with delandistrogene moxeparvovec. The data support efficacy of delandistrogene moxeparvovec in improving cardiac function and reversing adverse cardiac remodeling in DMD^{MDX} rats.

### Improved Ambulation and Vertical Movement

Movement of the DMD^{mdx} rats was measured in-life with laser monitoring in an open field activity cage. Ambulation and vertical activity (measurement of vertical movement) were measured based on the number of light beam breaks per hour. Diseased animals lose ambulation and vertical movement over time due to muscle damage. Activity cage was performed within two weeks of necropsy.

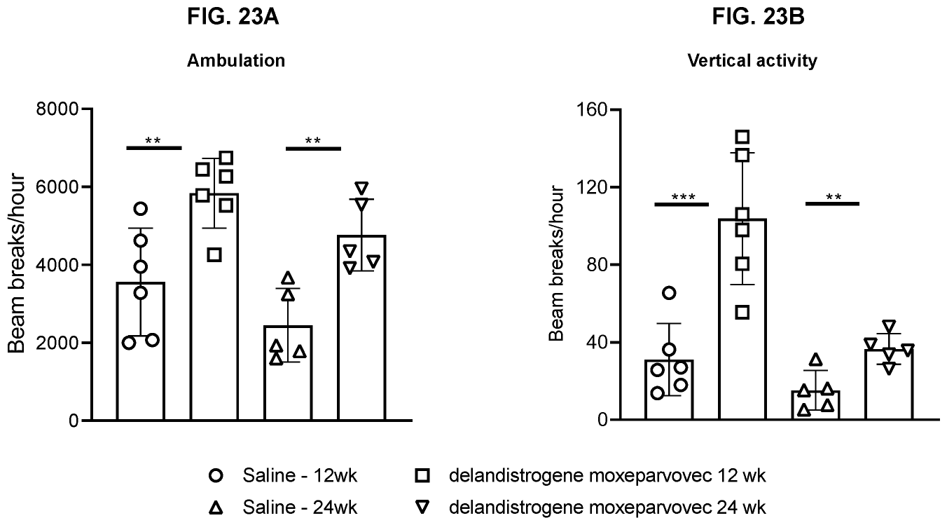

Movement, both ambulation and vertical activity, was significantly increased for all treated groups at all timepoints. A statistically significant increase in ambulation and vertical activity at 12-week post systemic delivery of delandistrogene moxeparvovec was demonstrated (FIGS. 23A and 23B). The same trend is seen at the 24-week timepoint.

A significant difference is seen in ambulation at 24 weeks (FIG. 23A) in the treated cohort vs. the saline cohort. The improvement in vertical activity at 24 weeks is less pronounced, but is still statistically significant compared to the saline treated age matched DMD^{MDX} cohort (FIG. 23B).

Altogether, this data show a significant functional improvement in mobility in the DMD^{MDX} rats after treatment with delandistrogene moxeparvovec vs, saline at both the 12- and 24-week timepoints.

### Serum Troponin Levels

Troponin I levels in serum were evaluated in a subset of the DMD^{mdx} rats one week (7 days) after dosing of 1.33×10¹⁴ delandistrogene moxeparvovec (n=6) vs. saline (n=6). Troponin I were evaluated in a different subset of animals 12 weeks post dosing at necropsy (n=5) vs. saline (n=5).

Troponin I measures the levels of troponin I proteins in the serum. These proteins are released when the heart muscle has been damaged, such as occurs with a heart attack. The more damage there is to the heart, the greater the amount of troponin I present in the serum. Consistent with a DMD animal model, troponin I elevation in the serum and cardiac disease is a characterization of the DMD^{MDX} rat model.

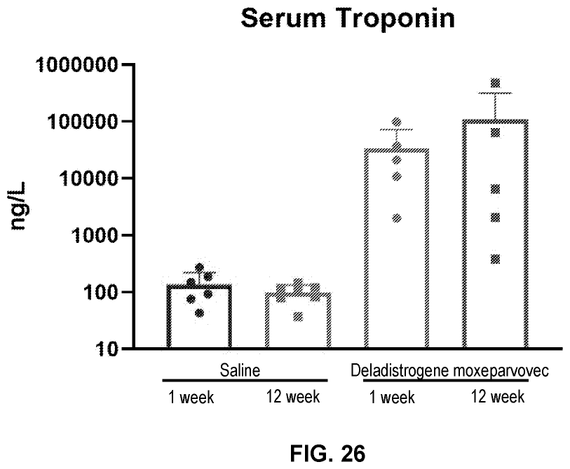

However, as shown in FIG. 26, no significant difference in serum troponin 1 blood levels was observed between the saline-treated and delandistrogene moxeparvovec-treated animals both 1 week post-dosing and 12 weeks post-dosing—this suggests no cardiac toxicity is caused by gene therapy treatment.

### CONCLUSIONS

This study was designed to test the efficacy and functional improvement of delandistrogene moxeparvovec treatment in the DMD^{MDX} rat model. The DMD^{MDX} rat model exhibits a more severe phenotype than the DMD^{MDX} mouse model and better models the cardiac and fibrotic phenotypes of DMD patients.

The in-life functional outcome of activity cage showed improvement with treatment with delandistrogene moxeparvovec. Both vertical activity and ambulation were significantly improved in treated animals compared to saline controls across all timepoints. Sustained functional improvement across all timepoints demonstrates durability of expression and function of systemic delivery of the clinically intended dose of 1.33×10¹⁴ vg/kg delandistrogene moxeparvovec.

Echocardiography showed improved indicators of cardiac disease with delandistrogene moxeparvovec systemic treatment, at both 12 and 24 weeks post treatment. Overall, this data demonstrate improvements in cardiac function with delandistrogene moxeparvovec treatment in the DMD^{MDX} rat model through 24 weeks post delivery.

Micro-dystrophin was expressed across muscles, including heart, in the DMD^{mdx} rat model, and this was associated with reduction in dystrophic pathogenesis and improved ambulation. Targeted expression of micro-dystrophin in heart reduced cardiac fibrosis and improved cardiac function, as evidenced by echocardiography. In sum, delandistrogene moxeparvovec has demonstrated targeted expression in skeletal and cardiac muscles, which corresponded with improved functional outcomes (e.g., cardiac) in an mdx rat model.

### REFERENCES

1. Hoffman, E. P., Brown, R. H., Jr. & Kunkel, L. M. Dystrophin: the protein product of the Duchenne muscular dystrophy locus. Cell 51, 919-928 (1987).
2. Straub, V. & Campbell, K. P. Muscular dystrophies and the dystrophin-glycoprotein complex. Curr Opin Neurol 10, 168-175 (1997).
3. Sacco, A., et al. Short telomeres and stem cell exhaustion model Duchenne muscular dystrophy in mdx/mTR mice. Cell 143, 1059-1071 (2010).
4. Wallace, G. Q. & McNally, E. M. Mechanisms of muscle degeneration, regeneration, and repair in the muscular dystrophies. Annu Rev Physiol 71, 37-57 (2009).
5. Zhou, L. & Lu, H. Targeting fibrosis in Duchenne muscular dystrophy. J Neuropathol Exp Neurol 69, 771-776 (2010).
6. Desguerre, I., et al. Endomysial fibrosis in Duchenne muscular dystrophy: a marker of poor outcome associated with macrophage alternative activation. J Neuropathol Exp Neurol 68, 762-773 (2009).
7. DiPrimio, N., McPhee, S. W. & Samulski, R. J. Adeno-associated virus for the treatment of muscle diseases: toward clinical trials. Curr Opin Mol Ther 12, 553-560 (2010).
8. Mendell, J. R., et al. Sustained alpha-sarcoglycan gene expression after gene transfer in limb-girdle muscular dystrophy, type 2D. Ann Neurol 68, 629-638 (2010).
9. Mendell, J. R., et al. Limb-girdle muscular dystrophy type 2D gene therapy restores alpha-sarcoglycan and associated proteins. Ann Neurol 66, 290-297 (2009).
10. Mendell, J. R., et al. A phase 1/2a follistatin gene therapy trial for becker muscular dystrophy. Molecular therapy: the journal of the American Society of Gene Therapy 23, 192-201 (2015).
11. Carnwath, J. W. & Shotton, D. M. Muscular dystrophy in the mdx mouse: histopathology of the soleus and extensor digitorum longus muscles. J Neurol Sci 80, 39-54 (1987).
12. Coulton, G. R., Morgan, J. E., Partridge, T. A. & Sloper, J. C. The mdx mouse skeletal muscle myopathy: I. A histological, morphometric and biochemical investigation. Neuropathol Appl Neurobiol 14, 53-70 (1988).
13. Cullen, M. J. & Jaros, E. Ultrastructure of the skeletal muscle in the X chromosome-linked dystrophic (mdx) mouse. Comparison with Duchenne muscular dystrophy. Acta Neuropathol 77, 69-81 (1988).
14. Dupont-Versteegden, E. E. & McCarter, R. J. Differential expression of muscular dystrophy in diaphragm versus hindlimb muscles of mdx mice. Muscle Nerve 15, 1105-1110 (1992).
15. Stedman, H. H., et al. The mdx mouse diaphragm reproduces the degenerative changes of Duchenne muscular dystrophy. Nature 352, 536-539 (1991).
16. Deconinck, A. E., et al. Utrophin-dystrophin-deficient mice as a model for Duchenne muscular dystrophy. Cell 90, 717-727 (1997).
17. Grady, R. M., et al. Skeletal and cardiac myopathies in mice lacking utrophin and dystrophin: a model for Duchenne muscular dystrophy. Cell 90, 729-738 (1997).
18. Love, D. R., et al. An autosomal transcript in skeletal muscle with homology to dystrophin. Nature 339, 55-58 (1989).
19. Tinsley, J. M., et al. Primary structure of dystrophin-related protein. Nature 360, 591-593 (1992).
20. Tinsley, J., et al. Expression of full-length utrophin prevents muscular dystrophy in mdx mice. Nat Med 4, 1441-1444 (1998).
21. Squire, S., et al. Prevention of pathology in mdx mice by expression of utrophin: analysis using an inducible transgenic expression system. Hum Mol Genet 11, 3333-3344 (2002).
22. Rafael, J. A., Tinsley, J. M., Potter, A. C., Deconinck, A. E. & Davies, K. E. Skeletal muscle-specific expression of a utrophin transgene rescues utrophin-dystrophin deficient mice. Nat Genet 19, 79-82 (1998).
23. Zhou, L., et al. Haploinsufficiency of utrophin gene worsens skeletal muscle inflammation and fibrosis in mdx mice. J Neurol Sci 264, 106-111 (2008).
24. Gutpell, K. M., Hrinivich, W. T. & Hoffman, L. M. Skeletal Muscle Fibrosis in the mdx/utrn+/− Mouse Validates Its Suitability as a Murine Model of Duchenne Muscular Dystrophy. PloS one 10, e0117306 (2015).
25. Rodino-Klapac, L. R., et al. Micro-dystrophin and follistatin co-delivery restores muscle function in aged DMD model. Human molecular genetics 22, 4929-4937 (2013).
26. Nevo, Y., et al. The Ras antagonist, famesylthiosalicylic acid (FTS), decreases fibrosis and improves muscle strength in dy/dy mouse model of muscular dystrophy. PloS one 6, e18049 (2011).
27. Rodino-Klapac, L. R., et al. A translational approach for limb vascular delivery of the micro-dystrophin gene without high volume or high pressure for treatment of Duchenne muscular dystrophy. J Transl Med 5, 45 (2007).
28. Mulieri, L. A., Hasenfuss, G., Ittleman, F., Blanchard, E. M. & Alpert, N. R. Protection of human left ventricular myocardium from cutting injury with 2,3-butanedione monoxime. Circ Res 65, 1441-1449 (1989).
29. Rodino-Klapac, L. R., et al. Persistent expression of FLAG-tagged micro dystrophin in nonhuman primates following intramuscular and vascular delivery. Molecular therapy: the journal of the American Society of Gene Therapy 18, 109-117 (2010).
30. Grose, W. E., et al. Homologous recombination mediates functional recovery of dysferlin deficiency following AAV5 gene transfer. PloS one 7, e39233 (2012).
31. Liu, M., et al. Adeno-associated virus-mediated microdystrophin expression protects young mdx muscle from contraction-induced injury. Mol Ther 11, 245-256 (2005).
32. Harper, S. Q., et al. Modular flexibility of dystrophin: implications for gene therapy of Duchenne muscular dystrophy. Nature medicine 8, 253-261 (2002).
33. Rodino-Klapac, L. R., et al. Persistent expression of FLAG-tagged micro dystrophin in nonhuman primates following intramuscular and vascular delivery. Mol Ther 18, 109-117 (2010).
34. Salva, M. Z., et al. Design of tissue-specific regulatory cassettes for high-level rAAV-mediated expression in skeletal and cardiac muscle. Mol Ther 15, 320-329 (2007).
35. Sondergaard, P. C., et al. AAV.Dysferlin Overlap Vectors Restore Function in Dysferlinopathy Animal Models. Annals of clinical and translational neurology 2, 256-270 (2015).
36. De, B. P., et al. High levels of persistent expression of alpha1-antitrypsin mediated by the nonhuman primate serotype rh.10 adeno-associated virus despite preexisting immunity to common human adeno-associated viruses. Mol Ther 13, 67-76 (2006).
37. Rodino-Klapac, L. R., et al. A translational approach for limb vascular delivery of the micro-dystrophin gene without high volume or high pressure for treatment of Duchenne muscular dystrophy. Journal of translational medicine 5, 45 (2007).
38. Bulfield et al., X chromosome-linked muscular dystrophy (mdx) in the mouse. Proc Natl Acad Sci USA. 1984; 81(4): 1189-1192.
39. Sicinski et al., The molecular basis of muscular dystrophy in the mdx mouse: a point mutation. Science. 1989 30; 244(4912):1578-80

1. A method of producing a recombinant adeno-associated virus (rAAV) rAAVrh74.MHCK7.microdystrophin in adherent mammalian cells by a suspension seed process, comprising:
(a) culturing cells with a first growth medium comprising serum in a N-2 container;
(b) removing the cells from the first medium;
(c) inoculating the cells from step (b) into a second medium comprising no serum or serum at a concentration less than the first medium in a N-1 container;
(d) culturing the cells in the N-1 container under suspension conditions; and
(e) inoculating a third medium in a bioreactor with the cells from step (d).

2. The method of claim 1, wherein the rAAV comprises the human micro-dystrophin nucleotide sequence of SEQ ID NO:1.

3. The method of claim 2, wherein the rAAV comprises the MHCK7 promoter sequence of SEQ ID NO: 7.

4. The method of claim 1, wherein the rAAV comprises the human micro-dystrophin nucleotide sequence of SEQ ID NO:1 and the MHCK7 promoter sequence of SEQ ID NO: 7.

5. The method of claim 1, wherein the suspension seed process further comprises:
(f) transfecting the adherent cells with a transgene plasmid comprising a rAAVrh74.MHCK7.microdystrophin construct, a plasmid comprising an AAV rep gene and an AAV cap gene, and an adenovirus helper plasmid.

6. The method of claim 5, wherein the transgene plasmid comprising a rAAVrh74.MHCK7.microdystrophin construct comprises:
the nucleic acid sequence of SEQ ID NO: 9;
nucleotides 55-5021 of SEQ ID NO: 3; or
nucleotides 1-4977 of SEQ ID NO: 8.

7. The method of claim 5, wherein the plasmid comprising an AAV rep gene and an AAV cap gene comprises an AAV2 rep gene and an rAAVrh74 cap gene.

8. The method of claim 5, wherein the adenovirus helper plasmid comprises an adenovirus 5 E2A, E4ORF6, and a VA RNA gene.

9. The method of claim 1, wherein the suspension seed process further comprises:
(g) lysing the adherent cells.

10. The method of claim 9, wherein the adherent cells are lysed by freeze-thaw, solid shear, hypertonic and/or hypotonic lysis, liquid shear, sonication, high-pressure extrusion, detergent lysis, or combinations thereof.

11. The method of claim 1, wherein the suspension seed process further comprises:
(h) purifying the rAAV by at least one column chromatography step.

12. The method of claim 11, wherein the at least one column chromatography step comprises an anion exchange chromatography, a size exclusion chromatography, or a combination thereof.

13.-14. (canceled)

15. The method of claim 1, wherein the bioreactor is an adherent bioreactor.

16. (canceled)

17. The method of claim 1, wherein the third medium in the bioreactor comprises at least one factor that promotes cell adherence.

18. The method of claim 17, wherein the at least one factor that promotes cell adherence is selected from the group consisting of serum, FBS, fibronectin, collagen, laminin, calcium ions, proteoglycans or non-proteoglycan polysaccharides of the extracellular matrix, and combinations thereof.

19. The method of claim 17, wherein the third medium in the bioreactor comprises DMEM and 10% FBS.

20. The method of claim 1, wherein the adherent cells are cultured under suspension conditions for about 48-72 hours.

21. The method of claim 1, wherein the N-1 container is a suspension shake flask.

22. The method of claim 1, wherein the adherent cells are selected from the group consisting of HeLa cells, CHO cells, HEK-293 cells, VERO cells, BHK cells, MDCK cells, MDBK cells, and COS cells.

23. The method of claim 22, wherein the adherent cells are HeLa cells or HEK-293 cells.

24. The method of claim 23, wherein the adherent cells are HEK-293 cells.

25. The method of claim 1, wherein the adherent cells are not suspension-adapted.

26. The method of claim 1, wherein culturing the cells under suspension conditions does not alter the adherent-dependency of the cells.

27. (canceled)

28. A composition comprising a recombinant adeno-associated virus (rAAV) rAAVrh74.MHCK7.microdystrophin, wherein the rAAV is made by the method of claim 1.

29. The composition of claim 28, wherein the composition comprises:
a) rAAV particles comprising the nucleic acid sequence of SEQ ID NO: 9;
b) rAAV particles comprising nucleotides 55-5021 of SEQ ID NO: 3; and/or
c) rAAV particles comprising nucleotides 1-4977 of SEQ ID NO: 8.

30. A method of treating muscular dystrophy in a human subject in need thereof comprising administering the composition of claim 29 to said human subject.

31. The method of claim 30, wherein the rAAV is administered using a systemic route of administration and at a dose of about 5.0×10¹² vg/kg to about 1.0×10¹⁵ vg/kg.

32. The method of claim 31, wherein the systemic route of administration is an intravenous route and the dose of the rAAV administered is about 2×10¹⁴ vg/kg.

33. The method of claim 30, wherein the dose of rAAV is administered at a concentration of about 10 mL/kg.

34. The method of claim 30, wherein the rAAV is administered by injection, infusion, or implantation.

35. The method of claim 34, wherein the rAAV is administered by infusion over approximately one hour.

36. The method of claim 30, wherein the rAAV is administered by an intravenous route through a peripheral limb vein.

37. The method of claim 30, wherein the muscular dystrophy is Duchenne muscular dystrophy or Becker's muscular dystrophy.

38. The method of claim 37, wherein the muscular dystrophy is Duchenne muscular dystrophy.

39. The method of claim 30, wherein the level of micro-dystrophin gene expression in a cell of the subject is increased after administration of the rAAV as compared to the level of micro-dystrophin gene expression before administration of the rAAV.

40. The method of claim 39, wherein expression of the micro-dystrophin gene in the cell is detected by measuring the micro-dystrophin protein level by Western blot in muscle biopsied before and after administration of the rAAV.

41. The method of claim 40, wherein the expression is at least 55.4% after administration of the rAAV, as compared to before.

42. The method of claim 30, wherein the mean percentage of micro-dystrophin positive fibers in the muscle tissue of the subject is increased after administration of the rAAV, as compared to the number of micro-dystrophin positive fibers before administration of the rAAV.

43. The method of claim 42, wherein the mean percentage of micro-dystrophin positive fibers is at least 70.5% and the mean intensity is at least 116.9% as detected by immunofluorescence (IF) in muscle biopsies before and after administration of the rAAV.

44. The method of claim 30, wherein micro-dystrophin transduction by vector genome count is at least 3.87 mean vector genome copies per nucleus.

45. The method of claim 30, wherein the composition is administered to a genotyped patient.

46. The method of claim 45, wherein the genotyped patient is genotyped for at least one mutation in exons 18-79 of the human dystrophin (DMD) gene.

47. The method of claim 45, further comprising genotyping the DMD gene of the human subject prior to administering the composition to said human subject.

48. The method of claim 47, wherein the genotyping detects at least one mutation in exons 18 to 79 of the DMD gene, wherein the at least one mutation is a frameshift deletion, a frameshift duplication, a premature stop, or other pathogenic variant resulting in the absence of expression of the human dystrophin protein.

49.-53. (canceled)
